(19) United States
(12) Patent Application Publication
(10) Pub. No.: US 2026/0209217 A1
(43) Pub. Date: Jul. 23, 2026

(54) CRYSTALLINE FORMS OF (S)-5-((3-(4-(4-FLUOROPHENYL)-1H-IMIDAZOL-2-YL)CHROMAN-6-YL)OXY)-3,4-DIHYDRO-1,8-NAPHTHYRIDIN-2(1H)-ONE

(71) Applicant: Jazz Pharmaceuticals Ireland Limited, Dublin (IE)

(72) Inventors: Adrian COOGAN, Maynooth (IE); Anthony MCMAHON, Dublin (IE); Andrew BELFIELD, Cheshire (GB); Clifford David JONES, Cheshire (GB); Fionn HURLEY, Dublin (IE)

(21) Appl. No.: 19/133,462

(22) PCT Filed: Nov. 29, 2023
(86) PCT No.: PCT/EP2023/083575

§ 371 Date: May 28, 2025

Related U.S. Application Data
(60) Provisional application No. 63/428,604, filed on Nov. 29, 2022.

(51) Int. Cl. C07D 471/04; A61K 31/4375

(52) U.S. Cl. CPC C07D 471/04; A61K 31/4375

(57) ABSTRACT

The present disclosure relates to crystalline forms of (S)-5-((3-(4-(4-fluorophenyl)-1H-imidazol-2-yl)chroman-6-yl)oxy)-3,4-dihydro-1,8-naphthyridin-2(1H)-one, Compound (I) or a salt, solvate, or solvate salt thereof.

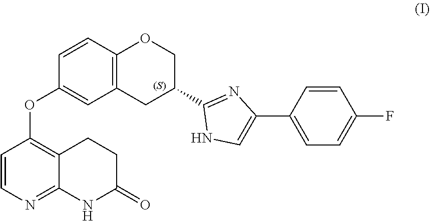

### CROSS-REFERENCE TO RELATED APPLICATIONS

The present Application claims priority to U.S. Provisional Patent Application No. 63/428,604 filed Nov. 29, 2022, and which is hereby incorporated by reference in its entirety.

### BACKGROUND

Mutations leading to uncontrolled signaling via the RAS-RAF-MAPK pathway are seen in more than one third of all cancers. RAF kinases (A-RAF, B-RAF and C-RAF) are an integral part of this pathway, with B-RAF mutations commonly seen in the clinic. Although most B-RAF V600E mutant skin cancers are sensitive to approved B-RAF selective drugs, B-RAF V600E mutant colorectal cancers are surprisingly insensitive to these agents as monotherapy due to the functions of other RAF family members and require combination therapy. B-RAF selective therapies fail to show clinical benefit against atypical B-RAF (non-V600E), other RAF and RAS driven tumors.

U.S. Pat. No. 10,183,939 discloses pan-RAF inhibitors that demonstrated binding affinity for B-RAF V600E and C-RAF, the disclosure of which is hereby incorporated by reference in its entirety. These pan-RAF inhibitors are identified as promising candidates in overcoming resistance mechanisms associated with clinically approved B-RAF selective drugs.

Crystallization of an active pharmaceutical ingredient can offer benefits for physiochemical properties. Such physicochemical properties include solubility, in vivo bioavailability, stability, melting point, flow properties, hygroscopicity, among others. Therefore, crystalline forms can be advantageous for pharmaceutical products. Thus, there is a need for crystalline forms of RAF inhibitors.

### SUMMARY

Aspects of the present disclosure relate to a crystalline form of Compound I

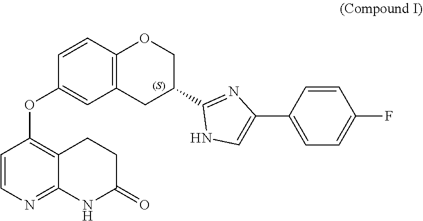

or a salt, solvate, or solvate salt thereof.

In embodiments, the crystalline form of Compound I is Form A, Form B, Form C, Form D, Form E, Form F, Form G, Form H, Form I, Form J, Form K, Form L, or Form M, or any combination thereof.

In embodiments, the crystalline form of Compound I is Form A.

In embodiments, Form A is characterized by peaks in an X-ray powder diffraction (XPRD) pattern at 5.8±0.2, 11.8±0.2, 16.6±0.2, and 23.1±0.2 °2θ. In embodiments Form A is further characterized by at least one peak in an XPRD pattern selected from 11.4±0.2, 17.3±0.2, 17.5±0.2, 17.9±0.2, and 24.2±0.2 °2θ. In embodiments, Form A is characterized by peaks in an XPRD pattern at 5.8±0.2, 6.0±0.2, 7.1±0.2, 9.3±0.2, 11.4±0.2, 11.8±0.2, 14.1±0.2, 16.6±0.2, 17.3±0.2, 17.5±0.2, 17.9±0.2, 18.5±0.2, 19.1±0.2, 19.8±0.2, 20.7±0.2, 21.3±0.2, 21.6±0.2, 22.1±0.2, 23.1±0.2, 24.2±0.2, 25.6±0.2, 26.1±0.2, 26.5±0.2, 27.3±0.2, 28.4±0.2, and 35.8±0.2 020. In embodiments, Form A is characterized by an XRPD pattern substantially similar to that shown in FIG. 1A.

In embodiments, Form A exhibits a Differential Scanning Calorimetry (DSC) thermogram comprising a peak at 77±5, 243±5, 271±5° C. or 285±5° C., or any combination thereof. In embodiments, Form A exhibits a Differential Scanning Calorimetry (DSC) thermogram comprising a peak at 77±5, 243±5, 245±5, 271±5° C. or 285±5° C., or any combination thereof. In embodiments, the peak at 67±5° C. is associated with an enthalpy of 61±5 Joule/gram (J/g); the peak at 239±5° C. is associated with an enthalpy of 78±5 J/g; the peak at 245±5° C. is associated with an enthalpy of 67±5 J/g; and the peak at 277±5° C. is associated with an enthalpy of 84±5 J/g. In embodiments, Form A exhibits a DSC thermogram comprising an endothermic peak with an onset at 46±5, 231±5, or 271±5° C., or any combination thereof. In embodiments, Form A exhibits a DSC thermogram comprising an exothermic peak with an onset at 242±5° C.

In embodiments, Form A exhibits a 3% weight loss at a temperature of 120±5° C., a 1% weight loss at 220±5° C. as measured by thermogravimetric (TGA) analysis, or both.

In embodiments, Form A is a hydrate.

In embodiments, the crystalline form of Compound I is Form B. In embodiments, Form B is characterized by peaks in an XPRD pattern at 6.2±0.2, 12.6±0.2, and 23.4±0.2 °2θ. In embodiments, Form B is characterized by an XRPD pattern substantially similar to that shown in FIG. 2A.

In embodiments, Form B exhibits a DSC thermogram comprising a peak at 71±5, or 170±5° C., or combination thereof. In embodiments, the peak at 71±5° C. is associated with an enthalpy of 107±5 J/g and the peak at 170±5° C. is associated with an enthalpy of 2±5 J/g. In embodiments, Form B exhibits a DSC thermogram comprising an endothermic peak with an onset at 33±5, or 165±5° C., or combination thereof.

In embodiments, Form B exhibits an 8% weight loss at a temperature of 110±5° C. as measured by TGA analysis.

In embodiments, Form B is a hydrate.

In embodiments, the crystalline form of Compound I is Form C.

In embodiments, Form C is characterized by peaks in an XPRD pattern at 16.8±0.2, 23.1±0.2, and 24.2±0.2 °2θ. In embodiments, Form C is further characterized by at least one peak an in XPRD pattern selected from 5.8±0.2, 11.5±0.2, 17.2±0.2, 17.5±0.2, and 18.4±0.2 °2θ. In embodiments, Form C is characterized by peaks in an XPRD pattern at 5.8±0.2, 7.1±0.2, 9.4±0.2, 11.5±0.2, 11.7±0.2, 12.8±0.2, 14.1±0.2, 16.8±0.2, 17.2±0.2, 17.5±0.2, 17.9±0.2, 18.2±0.2, 18.4±0.2, 19.0±0.2, 19.6±0.2, 20.4±0.2, 21.3±0.2, 22.1±0.2, 22.6±0.2, 23.1±0.2, 24.2±0.2, 25.7±0.2, 26.2±0.2, 26.6±0.2, 28.5±0.2, 30.3±0.2, 31.0±0.2, 32.4±0.2, 34.0±0.2, and 36.2±0.2 °2θ. In embodiments, Form C is characterized by an XRPD pattern substantially similar to that shown in FIG. 3A.

In embodiments, Form C exhibits a DSC thermogram comprising a peak at 92±5, 154±5, 231±5, or 242±5° C., or any combination thereof. In embodiments, the peak at 92±5° C. is associated with an enthalpy of 301±5 J/g; the peak at 154±5° C. is associated with an enthalpy of 63±5 J/g; the peak at 231±5° C. is associated with an enthalpy of 14±5 J/g; and the peak at 242±5° C. is associated with an enthalpy of 14±5 J/g. In embodiments, Form C exhibits a DSC thermogram comprising an endothermic peak with an onset at 56±5, 133±5, 229±5, or 239±5° C., or any combination thereof.

In embodiments, Form C exhibits a 16% weight loss at a temperature of 75±5° C. as measured by TGA analysis.

In embodiments, Form C is a solvate. In embodiments, solvate is a dimethyl sulfoxide (DMSO) solvate. In embodiments, solvate is a DMSO/water, dimethylacetamide (DMAc)/water, N-Methyl-2-pyrrolidone (NMP) and NMP/water solvate.

In embodiments, the crystalline form of Compound I is Form D.

In embodiments, Form D is characterized by peaks in an XPRD pattern at 12.5±0.2 and 23.4±0.2 °2θ. In embodiments, Form D is further characterized by at least one peak in an XPRD pattern selected from 11.5±0.2, 21.3±0.2, and 26.4±0.2 °2θ. Form D is characterized by peaks in an XPRD pattern at 6.1±0.2, 8.9±0.2, 21.3±0.2, 23.4±0.2, and 26.4±0.2 °2θ. In embodiments, Form D is characterized by an XRPD pattern substantially similar to that shown in FIG. 4A.

In embodiments, Form D exhibits a DSC thermogram comprising a peak at 84±5, or 191±5° C., or combination thereof. In embodiments, the peak at 84±5° C. is associated with an enthalpy of 300±5 J/g and the peak at 191±5° C. is associated with an enthalpy of 8±5 J/g. In embodiments, Form D exhibits a DSC thermogram comprising an endothermic peak with an onset at 52±5, or 179±5° C., or combination thereof.

In embodiments, Form D exhibits a 5% weight loss at a temperature of 110±5° C., a 2% weight loss at 170±5° C. as measured by TGA analysis, or both.

In embodiments, Form D is a solvate. In embodiments, the solvate is a dimethylformamide (DMF) solvate.

In embodiments, the crystalline form of Compound I is Form E.

In embodiments, Form E is characterized by peaks in an XPRD pattern at 5.8±0.2, 17.4±0.2, and 22.9±0.2 °2θ. In embodiments, Form E is further characterized by at least one peak an in XPRD pattern selected from 11.6±0.2, 16.7±0.2, 17.1±0.2, 23.3±0.2, and 24.0±0.2 °2θ. In embodiments, Form E is characterized by peaks in an XPRD pattern at 5.8±0.2, 7.0±0.2, 9.4±0.2, 11.4±0.2, 11.6±0.2, 14.1±0.2, 16.7±0.2, 17.1±0.2, 17.4±0.2, 17.8±0.2, 18.1±0.2, 18.3±0.2, 18.8±0.2, 19.4±0.2, 20.2±0.2, 20.9±0.2, 21.1±0.2, 21.8±0.2, 22.1±0.2, 22.9±0.2, 23.3±0.2, 24.0±0.2, 25.4±0.2, 25.9±0.2, 26.3±0.2, 28.3±0.2, 33.3±0.2, and 35.7±0.2 °2θ. In embodiments, Form E is characterized by an XRPD pattern substantially similar to that shown in FIG. 5A.

In embodiments, Form E exhibits a DSC thermogram comprising a peak at 176±5, 239±5, or 276±5° C., or any combination thereof. In embodiments, the peak at 176±5° C. is associated with an enthalpy of 75±5 J/g; the peak at 239±5° C. is associated with an enthalpy of 32±5 J/g; and the peak at 276±5° C. is associated with an enthalpy of 17±5 J/g. In embodiments, Form E exhibits a DSC thermogram comprising an endothermic peak with an onset at 139±5, 233±5, or 271±5° C., or any combination thereof.

In embodiments, Form E exhibits a 2% weight loss at a temperature of 80±5° C., a 16% weight loss at 140±5° C. as measured by TGA analysis, or both.

In embodiments, Form E is a solvate.

In embodiments, the crystalline form of Compound I is Form F.

In embodiments, Form F is characterized by peaks in an XPRD pattern at 13.0±0.2, 17.0±0.2, and 19.3±0.2 °2θ. In embodiments, Form F is further characterized by at least one peak in an XPRD pattern selected from 20.0±0.2, 22.5±0.2, and 23.3±0.2 °2θ. In embodiments, Form F is characterized by peaks in an XPRD pattern at 11.8±0.2, 13.0±0.2, 13.9±0.2, 14.2±0.2, 14.5±0.2, 15.8±0.2, 17.0±0.2, 19.3±0.2, 20.0±0.2, 21.0±0.2, 21.8±0.2, 22.1±0.2, 22.5±0.2, 23.3±0.2, 25.3±0.2, 25.5±0.2, 26.3±0.2, 26.8±0.2, 27.2±0.2, 27.9±0.2, 28.3±0.2, 30.4±0.2, 31.7±0.2, 32.2±0.2, 32.6±0.2, 35.9±0.2, and 39.5±0.2 °2θ. In embodiments, Form F is characterized by an XRPD pattern substantially similar to that shown in FIG. 6A.

In embodiments, Form F exhibits a DSC thermogram comprising a peak at 280±5° C. In embodiments, the peak at 277±5° C. is associated with an enthalpy of 113±5 J/g. In embodiments, Form F exhibits a DSC thermogram comprising an endothermic peak with an onset at 269±5° C.

In embodiments, Form F exhibits a weight loss of less than 1% at a temperature of 100±5° C. as measured by TGA.

In embodiments, Form F is anhydrous.

In embodiments, the crystalline form of Compound I is Form G.

In embodiments, Form G is characterized by peaks in an XPRD pattern at 12.6±0.2, 21.2±0.2, and 23.5±0.2 °2θ. In embodiments, Form G is further characterized by at least one peak in an XPRD pattern selected from 6.2±0.2, 13.4±0.2, 19.0±0.2, 26.2±0.2, and 26.5±0.2 °2θ. In embodiments, Form G is characterized by peaks in an XPRD pattern at 6.2±0.2, 11.4±0.2, 12.6±0.2, 13.4±0.2, 19.0±0.2, 21.2±0.2, 23.5±0.2, 26.2±0.2, 26.5±0.2, 28.0±0.2, 31.4±0.2, and 36.0±0.2 °2θ. In embodiments, Form G is characterized by an XRPD pattern substantially similar to that shown in FIG. 7A.

In embodiments, Form G is a solvate. In embodiments, the solvate is a DMSO solvate.

In embodiments, the crystalline form of Compound I is Form H.

In embodiments, Form H is characterized by peaks in an XRPD pattern at 5.7±0.2, 6.0±0.2, and 11.7±0.2 °2θ. In embodiments, Form H is further characterized by at least one peak an in XPRD pattern selected from 7.1±0.2, 11.4±0.2, 17.2±0.2, 23.0±0.2, and 24.0±0.2 °2θ. In embodiments, Form H is characterized by peaks in an XPRD pattern at 5.7±0.2, 6.0±0.2, 7.1±0.2, 9.2±0.2, 11.4±0.2, 11.7±0.2, 14.1±0.2, 16.5±0.2, 17.2±0.2, 17.8±0.2, 18.4±0.2, 19.1±0.2, 19.7±0.2, 20.1±0.2, 20.6±0.2, 22.0±0.2, 23.0±0.2, 24.0±0.2, 25.4±0.2, 26.0±0.2, and 28.2±0.2 °2θ. In embodiments, Form H is characterized by an XRPD pattern substantially similar to that shown in FIG. 8A.

In embodiments, Form H is anhydrous.

In embodiments, the crystalline form of Compound I is Form J.

In embodiments, Form J is characterized by peaks in an XRPD pattern at 11.1±0.2, 11.6±0.2, and 17.1±0.2 °2θ.

In embodiments, further characterized by at least one peak in an XRPD pattern selected from 5.6±0.2, 6.9±0.2, 16.2±0.2, 17.8±0.2, and 18.3±0.2 °2θ.

In embodiments, Form J is characterized by peaks in an XPRD pattern at 5.6±0.2, 6.9±0.2, 9.1±0.2, 11.1±0.2, 11.6±0.2, 12.9±0.2, 13.9±0.2, 16.2±0.2, 16.8±0.2, 17.1±0.2, 17.5±0.2, 17.8±0.2, 18.29±0.2, 19.2±0.2, 19.7±0.2, 20.6±0.2, 21.0±0.2, 21.6±0.2, 22.4±0.2, 22.9±0.2, 23.4±0.2, 23.9±0.2, 25.9±0.2, 26.2±0.2, 27.5±0.2, 27.7±0.2, 28.0±0.2, 29.0±0.2, 30.1±0.2, 31.0±0.2, 31.7±0.2, 32.9±0.2, 37.2±0.2, and 38.4±0.2 °2θ.

In embodiments, Form J is characterized by an XRPD pattern substantially similar to that shown in FIG. 10A.

In embodiments, Form J exhibits a DSC thermogram comprising a peak at 83±5° C., 196±5° C., and 285±5° C.

In embodiments, the crystalline form of Compound I is Form K.

In embodiment, Form K is characterized by peaks in an XRPD pattern at 5.4±0.2, 13.4±0.2, and 17.9±0.2 °2θ.

In embodiments, characterized by peaks in an XRPD pattern at 5.5±0.2, 7.3±0.2, 11.4±0.2, 12.0±0.2, 12.9±0.2, 13.4±0.2, 14.6±0.2, 16.1±0.2, 16.9±0.2, 17.4±0.2, 17.9±0.2, 18.4±0.2, 19.0±0.2, 19.9±0.2, 21.1±0.2, 21.5±0.2, 21.7±0.2, 21.9±0.2, 22.2±0.2, 22.9±0.2, 23.2±0.2, 24.1±0.2, 24.8±0.2, 25.1±0.2, 27.8±0.2, 26.3±0.2, 26.7±0.2, 27.3±0.2, 27.6±0.2, 28.5±0.2, 29.7±0.2, 32.5±0.2, 33.9±0.2, 34.7±0.2, 35.3±0.2, 37.3±0.2, and 38.3±0.2 °2θ.

In embodiments, Form K is characterized by an XRPD pattern substantially similar to that shown in FIG. 13.

In embodiments, the crystalline form of Compound I is Form L.

In embodiments, Form L is characterized by peaks in an XRPD pattern at 4.3±0.2, 8.2±0.2, and 8.6±0.2 °2θ.

In embodiments, Form L is characterized by peaks in an XRPD pattern at 4.3±0.2, 8.2±0.2, 8.6±0.2, 12.9±0.2, 13.4±0.2, 15.4±0.2, 15.9±0.2, 17.2±0.2, 17.7±0.2, 18.2±0.2, 18.6±0.2, 19.2±0.2, 20.5±0.2, 21.5±0.2, 22.2±0.2, 24.1±0.2, 24.9±0.2, 25.7±0.2, 26.0±0.2, 26.6±0.2, 27.1±0.2, 27.5±0.2, 28.1±0.2, 29.2±0.2, 30.2±0.2, 31.2±0.2, and 38.5±0.2 °2θ.

In embodiments, Form L is characterized by an XRPD pattern substantially similar to that shown in FIG. 12.

In embodiments, the crystalline form of Compound I is Form M.

In embodiments, Form M is characterized by peaks in an XRPD pattern at 7.3±0.2, 10.4±0.2, and 12.7±0.2 °2θ.

In embodiments, Form M is characterized by peaks in an XRPD pattern at 7.3±0.2, 9.2±0.2, 10.4±0.2, 11.8±0.2, 12.7±0.2, 14.8±0.2, 16.2±0.2, 17.5±0.2, 18.6±0.2, 19.0±0.2, 19.9±0.2, 21.0±0.2, 22.3±0.2, 23.6±0.2, 29.2±0.2, 31.2±0.2, 32.5±0.2, and 33.5±0.2 °2θ.

In embodiments, Form M is characterized by an XRPD pattern substantially similar to that shown in FIG. 13.

In embodiments, the present disclosure provides compositions comprising a crystalline form of Compound 1 and a pharmaceutically acceptable excipient.

### BRIEF DESCRIPTION OF THE DRAWINGS

FIG. 1A shows the XRPD diffractogram of a sample of the crystalline Form A of Compound I.

FIG. 1B shows a differential scanning calorimetry (DCS) plot of the crystalline Form A of Compound I.

FIG. 1C shows thermogravimetric analysis (TGA) plot of the crystalline Form A of Compound I.

FIG. 1D shows dynamic vapor sorption (DVS) plot of the crystalline Form A of Compound I.

FIG. 1E shows the XRPD spectrum of a sample of the crystalline Form A* of Compound I.

FIG. 1F shows a DCS plot of the crystalline Form A* of Compound I.

FIG. 2A shows the XRPD diffractogram of a sample of the crystalline Form B of Compound I.

FIG. 2B shows a DCS plot of the crystalline Form B of Compound I.

FIG. 2C shows TGA plot of the crystalline Form B of Compound I.

FIG. 3A shows the XRPD diffractogram of a sample of the crystalline Form C of Compound I.

FIG. 3B shows a DCS plot of the crystalline Form C of Compound I.

FIG. 3C shows TGA plot of the crystalline Form C of Compound I.

FIG. 4A shows the XRPD diffractogram of a sample of the crystalline Form D of Compound I.

FIG. 4B shows a DCS plot of the crystalline Form D of Compound I.

FIG. 4C shows TGA plot of the crystalline Form D of Compound I.

FIG. 5A shows the XRPD diffractogram of a sample of the crystalline Form E of Compound I.

FIG. 5B shows a DCS plot of the crystalline Form E of Compound I.

FIG. 5C shows TGA plot of the crystalline Form E of Compound I.

FIG. 6A shows the XRPD diffractogram of a sample of the crystalline Form F of Compound I.

FIG. 6B shows a DCS plot of the crystalline Form F of Compound I.

FIG. 6C shows TGA plot of the crystalline Form F of Compound I.

FIG. 6D shows DVS plot of the crystalline Form F of Compound I.

FIG. 7 shows the XRPD diffractogram of a sample of the crystalline Form G of Compound I.

FIG. 8 shows the XRPD diffractogram of a sample of the crystalline Form H of Compound I.

FIG. 9A shows the XRPD diffractogram of a sample of the crystalline Form I of Compound I.

FIG. 9B shows a DCS plot of the crystalline Form I of Compound I.

FIG. 10A shows the XRPD diffractogram of a sample of the crystalline Form J of Compound I.

FIG. 10B shows a DCS plot of the crystalline Form J of Compound I.

FIG. 11 shows the XRPD diffractogram of a sample of the crystalline Form K of Compound I.

FIG. 12 shows the XRPD diffractogram of a sample of the crystalline Form L of Compound I.

FIG. 13 shows the XRPD diffractogram of a sample of the crystalline Form M of Compound I.

FIGS. 14A-14B show dissolution data for 10 mg capsules comprising Form A (FIG. 14A), capsules comprising Form A and 10% Form F (FIG. 14B), and capsules comprising Form F (FIG. 14C).

FIGS. 15A-15B show dissolution data for 50 mg capsules comprising Form A (FIG. 15A), capsules comprising Form A and 10% Form F (FIG. 15B), and capsules comprising Form F (FIG. 15C).

### DETAILED DESCRIPTION

The present disclosure relates to crystalline forms of RAF inhibitors to include crystalline Forms A, B, C, D, E, F, G, H, I, J, K, L and M. Crystalline Forms A and F exhibited a high degree of crystallinity and stability relative to Crystalline Forms C, D, E, F, G, H, I, J, K, L and M, and Crystalline Forms A and F are suitable for large scale manufacturing, indicating that Forms A and F have the desired characteristics for further pharmaceutical development.

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

### Definitions

While the following terms are believed to be well understood by one of ordinary skill in the art, the following definitions are set forth to facilitate explanation of the presently disclosed subject matter.

Compound I is (S)-5-((3-(4-(4-fluorophenyl)-1H-imidazol-2-yl)chroman-6-yl)oxy)-3,4-dihydro-1,8-naphthyridin-2(1H)-one having the structure shown below. In embodiments, Compound I is a pan-RAF inhibitor.

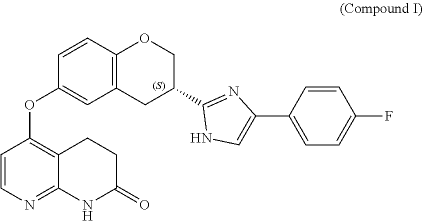

As used herein, the term “substantially” means greater than 85% (i.e., greater than 85, 86, 87, 88, 89, 90, 91, 92, 93, 94, 95, 96, 97, 98, 99, or 100%).

The term “substantially similar” as used herein with regards to an analytical spectrum, such as an XRPD pattern, means that a spectrum resembles the reference spectrum in both the peak locations and their relative intensities, allowing for variability appropriate in the art. For example, two spectra may be regarded as “substantially similar” when the two spectra share defining characteristics sufficient to differentiate them from a spectrum obtained for a different solid form. In embodiments, spectra or characterization data that are substantially similar to those of a reference crystalline form are understood by those of ordinary skill in the art to correspond to the same crystalline form as the particular reference. In analyzing whether spectra or characterization data are substantially similar, a person of ordinary skill in the art understands that particular characterization data points may vary to a reasonable extent while still describing a given solid form, due to, for example, experimental error and routine sample-to-sample analysis.

Polymorphism can be characterized as the ability of a compound to crystallize into different crystal forms, while maintaining the same chemical formula. A crystalline polymorph of a given drug substance is chemically identical to any other crystalline polymorph of that drug substance in containing the same atoms bonded to one another in the same way, but differs in its lattice arrangement, which can affect one or more physical properties, such as stability, solubility, melting point, bulk density, flow properties, bioavailability, etc.

The term “effective” is used to describe an amount of a compound, composition or component which, when used within the context of its intended use, effects an intended result. The term “effective” subsumes all other effective amounts or effective concentration terms, which are otherwise described or used in the present application.

The phrase “therapeutically effective amount” and the like, as used herein, indicate an amount necessary to administer to a patient, or to a cell, tissue, or organ of a patient, to achieve a therapeutic effect, such as an ameliorating or a curative effect in treating cancer. The therapeutically effective amount is sufficient to elicit the biological or medical response of a cell, tissue, system, animal, or human that is being sought by a researcher, veterinarian, medical doctor, or clinician.

All XRPD peaks and patterns are given in °2θ using Cu Kα1 radiation at a wavelength of 1.5406 A. The values of degree 20 allow appropriate error margins. For example, the degree 20 of “17.48±0.2” denotes a range from 17.46 to 17.50 degree 20. Depending on the sample preparation techniques, the calibration techniques applied to the instruments, human operational variation, and etc., those skilled in the art recognize that the appropriate error of margins for a XRPD can be ±0.2, which includes any value below ±0.2 such as ±0.1.

TGA and DSC thermograms for a given crystalline form of the same compound can vary within a margin of error. The values of a single peak, expressed in degree Celsius, allow appropriate error margins. Typically, the error margins are represented by “±”. For example, the single peak characteristic value of “120±5” denotes a range from 115 to 125. Depending on the sample preparation techniques, the calibration techniques applied to the instruments, human operational variations, and etc., those skilled in the art recognize that the appropriate error of margins for a single peak characteristic value can be ±5, which includes any value below ±5 such as ±4, ±3.5, +3, +2.5; +2.0; +1.5; +1.0; ±0.5; or less.

The following description includes information that may be useful in understanding the present invention. It is not an admission that any of the information provided herein is prior art or relevant to the presently claimed inventions, or that any publication specifically or implicitly referenced is prior art.

### Crystalline Forms of Compound I

Aspects of the present disclosure relate to crystalline forms of Compound I or salt, solvate, or solvate salt thereof. In embodiments, the crystalline form of Compound I is Form A, Form B, Form C, Form D, Form E, Form F, Form G, Form H, or any combination thereof.

In embodiments, the crystalline form of Compound I (e.g., Form A, Form B, Form C, Form D, Form E, Form F, Form G, Form H, Form I, Form J, Form K, Form L, and/or Form M) is a salt. Examples of salts include, but are not limited to, hydrochloric acid salt, maleic acid salt, fumaric acid salt, citric acid salt, malic acid salt, sulfuric acid salt, acetic acid salt, phosphoric acid salt, L-(+)-tartaric acid salt, D-glucuronic acid salt, benzoic acid salt, succinic acid salt, ethane sulfonic acid salt, methane sulfonic acid salt, p-toluene sulfonic acid salt, malonic acid salt, benzene sulfonic acid salt, and 1-hydroxy-2-naphthoic acid salt.

In embodiments, the crystalline form of Compound I (e.g., Form A, Form B, Form C, Form D, Form E, Form F, Form G, Form H, Form I, Form J, Form K, Form L, and/or Form M) is a solvate. Non-limiting examples of suitable solvates include dimethylformamide (DMF), acetone, dimethyl sulfoxide (DMSO), methanol, isopropanol, ethanol, and ethyl acetate.

In embodiments, the crystalline form of Compound I (e.g., Form A, Form B, Form C, Form D, Form E, Form F, Form G, Form H, Form I, Form J, Form K, Form L, and/or Form M) is an anhydrate.

In embodiments, the crystalline form of Compound I (e.g., Form A, Form B, Form C, Form D, Form E, Form F, Form G, Form H, Form I, Form J, Form K, Form L, and/or Form M) is a hydrate. Non-limiting examples of hydrates include hemihydrates, monohydrates, dihydrates, trihydrates, hexahydrates.

In embodiments, the crystalline form of Compound I may comprise of a mixture of one or more forms Compound I (e.g., Form A, Form B, Form C, Form D, Form E, Form F, Form G, Form H, Form I, Form J, Form K, Form L, and/or Form M). In some embodiments, the crystalline form of Compound I may comprise of substantially pure form of one form of Compound I.

In embodiments, the crystalline form of Compound I may comprise of over about 99.9%, about 99.8%, about 99.7%, about 99.6%, about 99.5%, about 99.4%, about 99.3%, about 99.2%, about 99.1%, or about 99.0% of one form of Compound I (e.g., Form A, Form B, Form C, Form D, Form E, Form F, Form G, Form H, Form I, Form J, Form K, Form L, and/or Form M).

In embodiments, the crystalline form of Compound I may comprise over about 99%, 98%, 97%, 96%, 95%, 94%, 93%, 92%, 91%, or 90% of one form of Compound I. In some embodiments, the crystalline form of Compound I may comprise over about 90%, 85%, 80%, 75%, 70%, 65%, 60%, 55%, 50%, 45%, or 40% of one form of Compound I (e.g., Form A, Form B, Form C, Form D, Form E, Form F, Form G, Form H, Form I, Form J, Form K, Form L, and/or Form M).

### I. Form A

In embodiments, the crystalline form of Compound I is Form A.

In embodiments, Form A is characterized by peaks in a XRPD pattern at 5.8±0.2, 11.8±0.2, 23.1±0.2, and 24.2±0.2 °2θ. In embodiments, the variance at any of these peaks is +0.1 °2θ.

In embodiments, Form A is characterized by peaks in a XRPD pattern at 11.8±0.2, 23.1±0.2, and 24.2±0.2 °2θ and least one (e.g., 1, 2, 3, 4, or 5) peak in an XRPD pattern selected from 5.8±0.2, 11.4±0.2, 17.3±0.2, 17.5±0.2, and 17.9±0.2 °2θ. In embodiments, the variance at any of these peaks is +0.1 °2θ.

In embodiments, Form A is characterized by peaks in a XRPD pattern at 5.8±0.2, 6.0±0.2, 7.1±0.2, 9.3±0.2, 11.4±0.2, 11.8±0.2, 14.1±0.2, 16.6±0.2, 17.3±0.2, 17.5±0.2, 17.9±0.2, 18.5±0.2, 19.1±0.2, 19.8±0.2, 20.7±0.2, 21.3±0.2, 21.6±0.2, 22.1±0.2, 23.1±0.2, 24.2±0.2, 25.6±0.2, 26.1±0.2, 26.5±0.2, 27.3±0.2, 28.4±0.2, and 35.8±0.2 °2θ. In embodiments, the variance at any of these peaks is +0.1 °2θ.

In embodiments, Form A is characterized by peaks in a XRPD pattern at 11.8±0.1, 23.1±0.1, and 24.2±0.1 °2θ.

In embodiments, Form A is characterized by peaks in a XRPD pattern at 11.8±0.1, 23.1±0.1, and 24.2±0.1 °2θ and least one (e.g., 1, 2, 3, 4, or 5) peak in an XRPD pattern selected from 5.8±0.1, 11.4±0.1, 17.3±0.1, 17.5±0.1, and 17.9±0.1 °2θ.

In embodiments, Form A is characterized by peaks in a XRPD pattern at 5.8±0.1, 6.0±0.1, 7.1±0.1, 9.3±0.1, 11.4±0.1, 11.8±0.1, 14.1±0.1, 16.6±0.1, 17.3±0.1, 17.5±0.1, 17.9±0.1, 18.5±0.1, 19.1±0.1, 19.8±0.1, 20.7±0.1, 21.3±0.1, 21.6±0.1, 22.1±0.1, 23.1±0.1, 24.2±0.1, 25.6±0.1, 26.1±0.1, 26.5±0.1, 27.3±0.1, 28.4±0.1, and 35.8±0.1 °2θ.

In embodiments, Form A is characterized by at least one peak in a XRPD pattern selected from 5.8±0.2, 7.1±0.2, 9.3±0.2, 11.4±0.2, 11.8±0.2, 14.1±0.2, 16.6±0.2, 17.3±0.2, 17.5±0.2, 17.9±0.2, 18.5±0.2, 20.7±0.2, 21.3±0.2, 21.6±0.2, 22.1±0.2, 24.2±0.2, 25.6±0.2, 27.3±0.2, 28.4±0.2, and 35.8±0.2 °2θ, absent in Form B, described herein.

In embodiments, Form A is characterized by at least one peak in a XRPD pattern selected from 5.8±0.2 or 27.3±0.2 °2θ, absent in Form C, described herein.

In embodiments, Form A is characterized by at least one peak in a XRPD pattern selected from 9.3±0.2, 14.1±0.2, 20.7±0.2, 22.1±0.2, 25.6±0.2, 27.3±0.2, 28.4±0.2, or 35.8±0.2 020, absent in Form D, described herein.

In embodiments, Form A is characterized by a peak in a XRPD pattern at 27.3±0.2 020, absent in Form E, described herein.

In embodiments, Form A is characterized by at least one peak in a XRPD pattern selected from 5.8±0.2, 6.0±0.2, 7.1±0.2, 9.3±0.2, 17.5±0.2, 17.9±0.2, or 18.5±0.2 °2θ, absent in Form F, described herein.

In embodiments, Form A is characterized by at least one peak in a XRPD pattern selected from 7.1±0.2, 9.3±0.2, 14.1±0.2, 17.3±0.2, 17.5±0.2, 17.9±0.2, 18.5±0.2, 19.8±0.2, 20.7±0.2, 22.1±0.2, 23.1±0.2, 24.2±0.2 25.6±0.2, or 27.3±0.2 °2θ, absent in Form G, described herein.

In embodiments, Form A is characterized by at least one peak in a XRPD pattern selected from 17.9±0.2, 26.5±0.2, or 35.8±0.2 °2θ, absent in Form H, described herein.

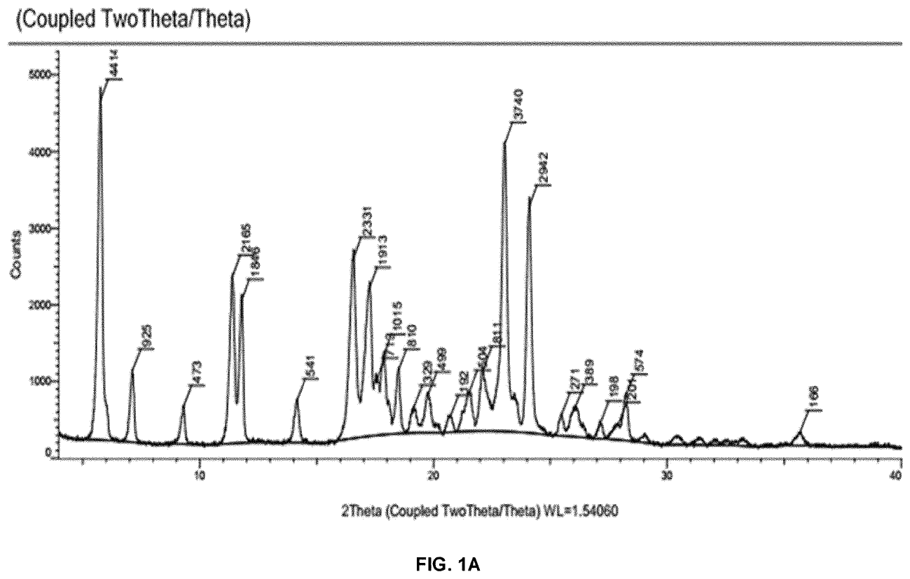

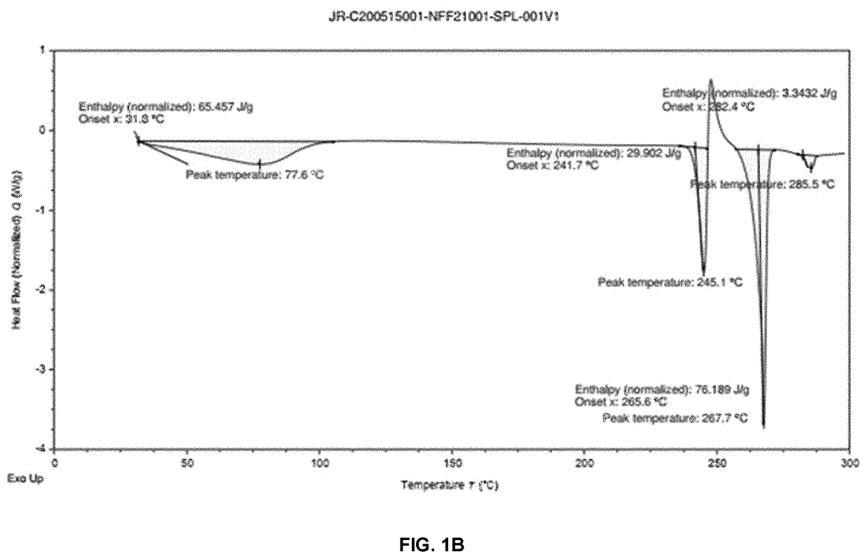

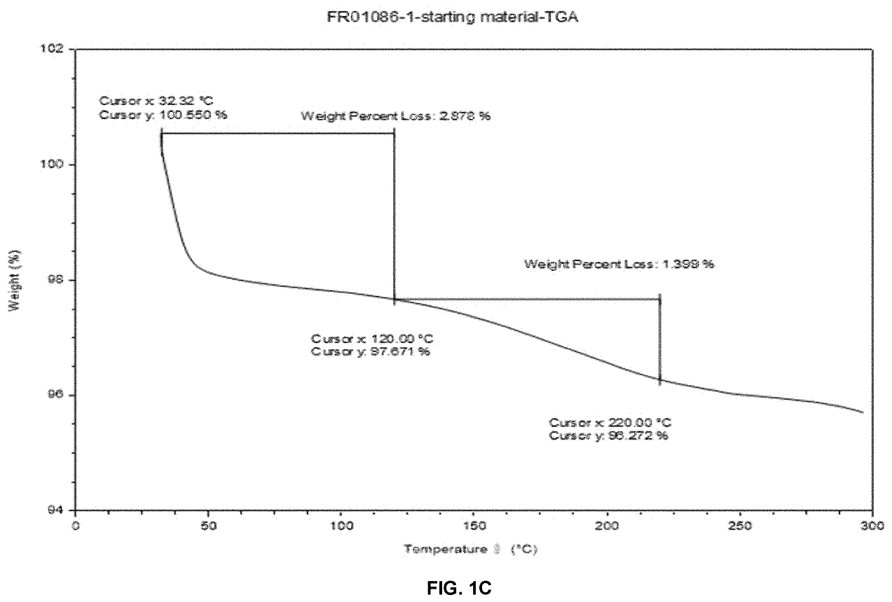

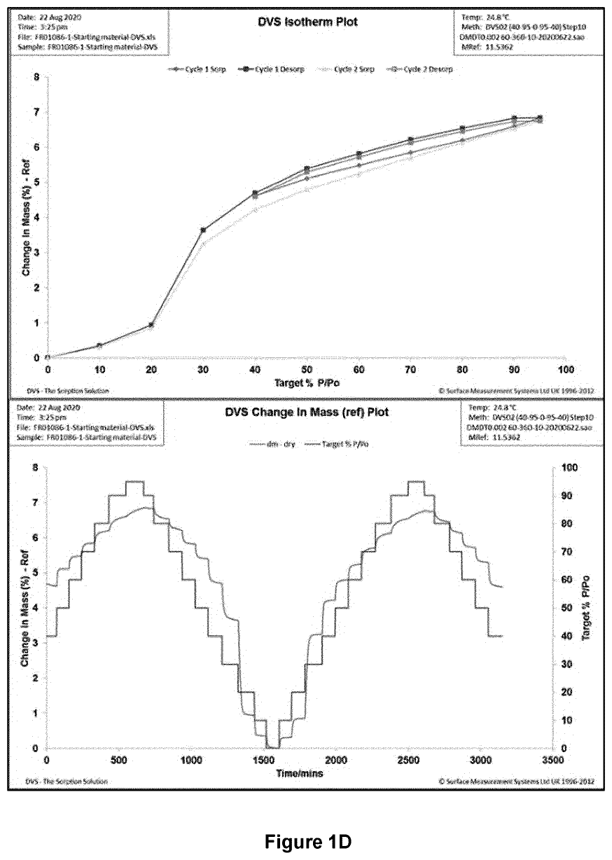

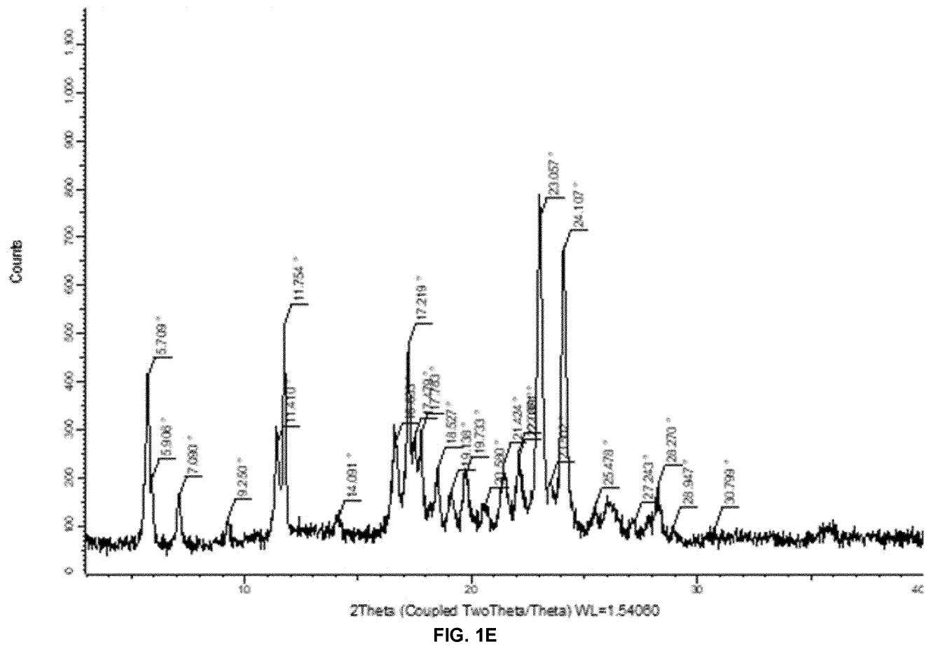

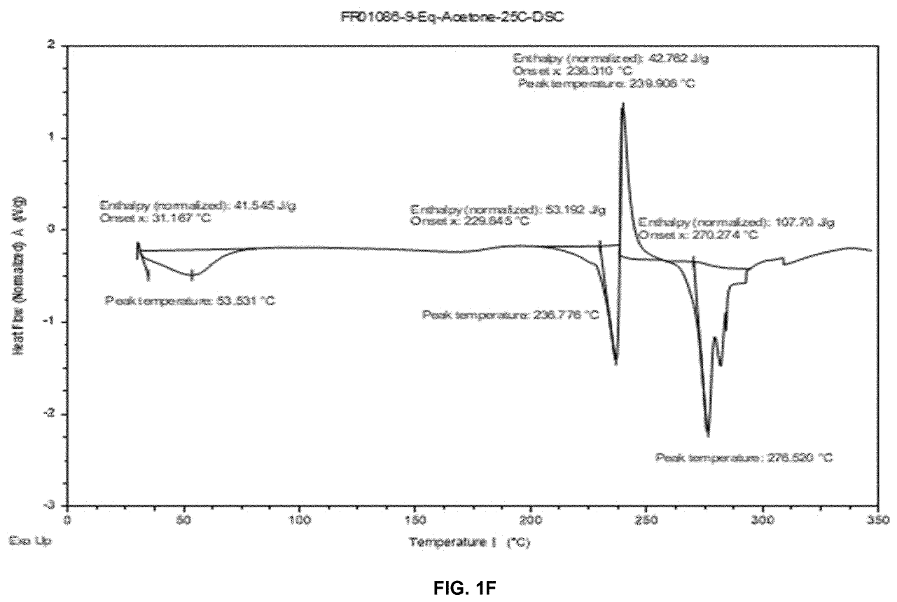

In embodiments, Form A is characterized by an XRPD pattern substantially similar to that shown in FIG. 1A.

In embodiments, Form A is characterized by an XRPD pattern comprising peaks show in Table 1.

In embodiments, Form A is characterized by have one, two, three, four, five, six, seven, eight, nine, ten, eleven, or more, XRPD peaks listed in Table 1.

In embodiments, Form A exhibits a DSC thermogram comprising a peak at 67±5, 239±5, 245±5, or 277±5° C., or any combination thereof. In embodiments, the peak at 67±5° C. is associated with an enthalpy of 61±5 Joule/gram (J/g); the peak at 239±5° C. is associated with an enthalpy of 78±5 J/g; the peak at 245±5° C. is associated with an enthalpy of 67±5 J/g; and the peak at 277±5° C. is associated with an enthalpy of 84±5 J/g.

In embodiments, Form A exhibits a DSC thermogram comprising an endothermic peak with an onset at 46±5, 231±5, or 271±5° C., or any combination thereof. In embodiments, Form A exhibits a DSC thermogram comprising an exothermic peak with an onset at 242±5° C.

In embodiments, Form A exhibits a DSC thermogram comprising a peak at 67±3, 239±3, 245±3, or 277±3° C., or any combination thereof. In embodiments, the peak at 67±5° C. is associated with an enthalpy of 61±5 J/g; the peak at 239±3° C. is associated with an enthalpy of 78±5 J/g; the peak at 245±3° C. is associated with an enthalpy of 67±5 J/g; and the peak at 277±3° C. is associated with an enthalpy of 84±5 J/g.

In embodiments, Form A exhibits a DSC thermogram comprising an endothermic peak with an onset at 46±3, 231±3, or 271±3° C., or any combination thereof. In embodiments, Form A exhibits a DSC thermogram comprising an exothermic peak with an onset at 242±3° C.

In embodiments, Form A exhibits a DSC thermogram substantially similar to that shown in FIG. 1B.

In embodiments, Form A exhibits about a 1% to about a 10% weight loss at a temperature of 120±5° C. as measured by thermogravimetric analysis (TGA). For example, Form A exhibits about a 1%, about 2%, about a 3%, about a 4%, about a 5%, about a 6%, about a 7%, about an 8%, about a 9%, or about a 10%, including all values therebetween, weight loss at a temperature of 120±5° C. as measured by TGA.

In embodiments, Form A exhibits about a 0.01% to about 5% weight loss at 220±5° C. as measured by TGA analysis. For example, Form A exhibits about a 0.01%, about 0.1%, about a 1%, about a 1.5%, about a 2%, about a 2.5%, about a 3%, about a 3.5%, about a 4%, about a 4.5%, or about a 5%, including all values therebetween, weight loss at a temperature of 220±5° C. as measured by TGA.

In embodiments, Form A exhibits substantially no weight loss at a temperature of 220±5° C. as measured by TGA.

In embodiments, Form A exhibits about a 1% to about a 10% weight loss at a temperature of 120±5° C., about a 0.01% to about 5% weight loss at 220±5° C. as measured by TGA analysis, or both.

In embodiments, Form A exhibits about a 3% weight loss at a temperature of 120±3° C., a 1% weight loss at 220±3° C. as measured by TGA analysis, or both.

In embodiments, Form A exhibits a TGA thermogram substantially similar to that shown in FIG. 1C.

### II. Form B

In embodiments, the crystalline form of Compound I is Form B.

In embodiments, Form B is characterized by a peak in a XRPD pattern at 39.1±0.2 °2θ.

In embodiments, Form B is characterized by peaks in an XRPD pattern at 12.7±0.2, 13.4±0.2, and 23.4±0.2 °2θ. In embodiments, the variance at any of these peaks is +0.1 °2θ.

In embodiments, the crystalline form of Compound I is Form B. In embodiments, Form B is characterized by peaks in an XPRD pattern at 6.2±0.2, 12.6±0.2, and 23.4±0.2 °2θ. In embodiments, the variance at any of these peaks is +0.1 °2θ.

In embodiments, Form B is characterized by peaks in an XRPD pattern at 12.7±0.2, 13.4±0.2, and 23.4±0.2 °2θ and at least one peak (e.g., 1, 2, 3, 4, or 5) in an XRPD pattern selected from 6.2±0.2, 13.6±0.2, 23.3±0.2, 26.1±0.2, and 26.3±0.2 °2θ. In embodiments, the variance at any of these peaks is +0.1 °2θ.

In embodiments, Form B is characterized by peaks in an XRPD pattern at 6.2±0.2, 12.7±0.2, 13.4±0.2, 13.6±0.2, 19.0±0.2, 19.8±0.2, 23.3±0.2, 23.4±0.2, 26.1±0.2, 26.3±0.2, and 39.1±0.2 °2θ. In embodiments, the variance at any of these peaks is +0.1 °2θ.

In embodiments, Form B is characterized by peaks in an XRPD pattern at 12.7±0.1, 13.4±0.1, and 23.4±0.1 °2θ.

In embodiments, Form B is characterized by peaks in an XRPD pattern at 12.7±0.1, 13.4±0.1, and 23.4±0.1 °2θ and at least one peak (e.g., 1, 2, 3, 4, or 5) in an XRPD pattern selected from 6.2±0.1, 13.6±0.1, 23.3±0.1, 26.1±0.1, and 26.3±0.1 °2θ.

In embodiments, Form B is characterized by peaks in an XRPD pattern at 6.2±0.1, 12.7±0.1, 13.4±0.1, 13.6±0.1, 19.0±0.1, 19.8±0.1, 23.3±0.1, 23.4±0.1, 26.1±0.1, 26.3±0.1, and 39.1±0.1 °2θ.

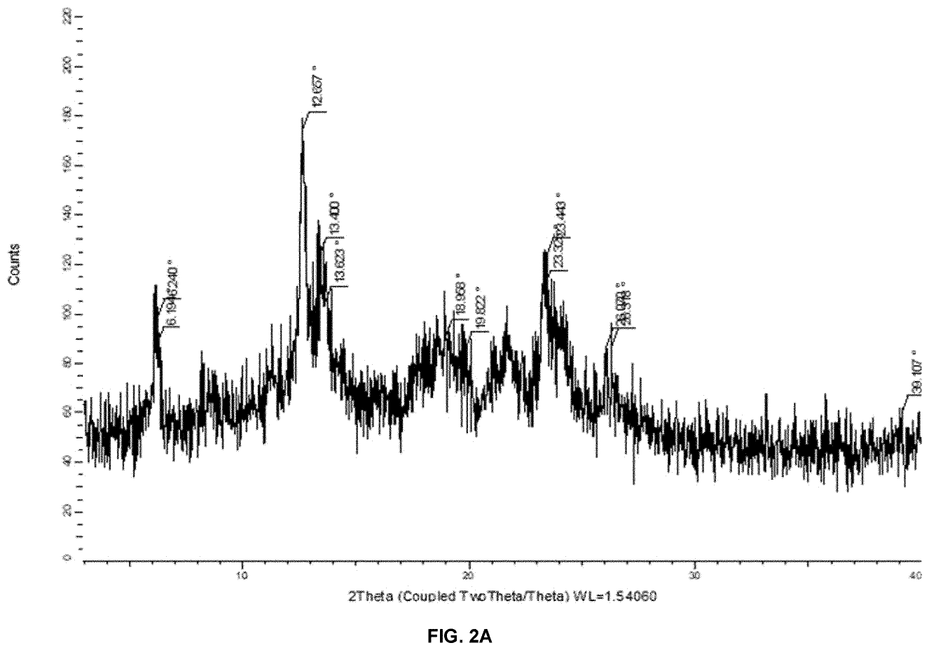

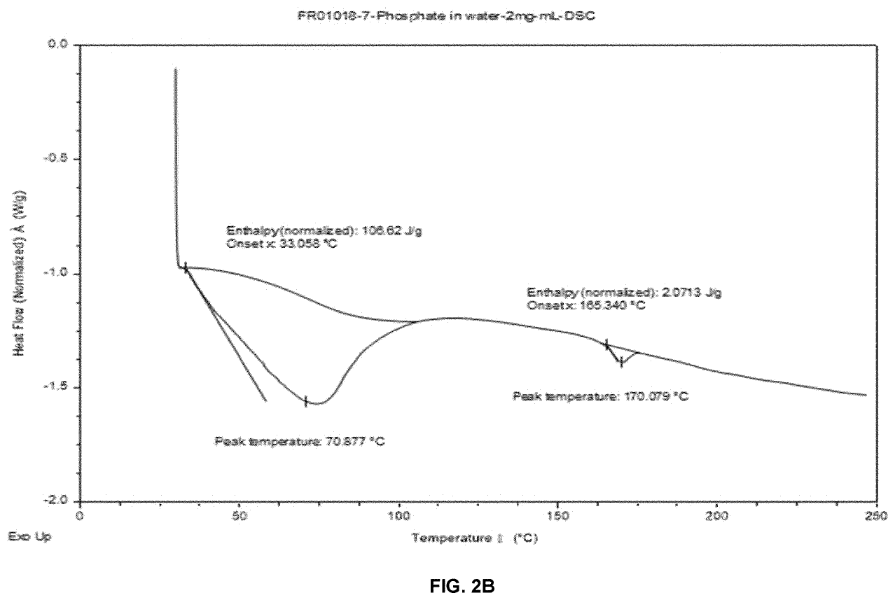

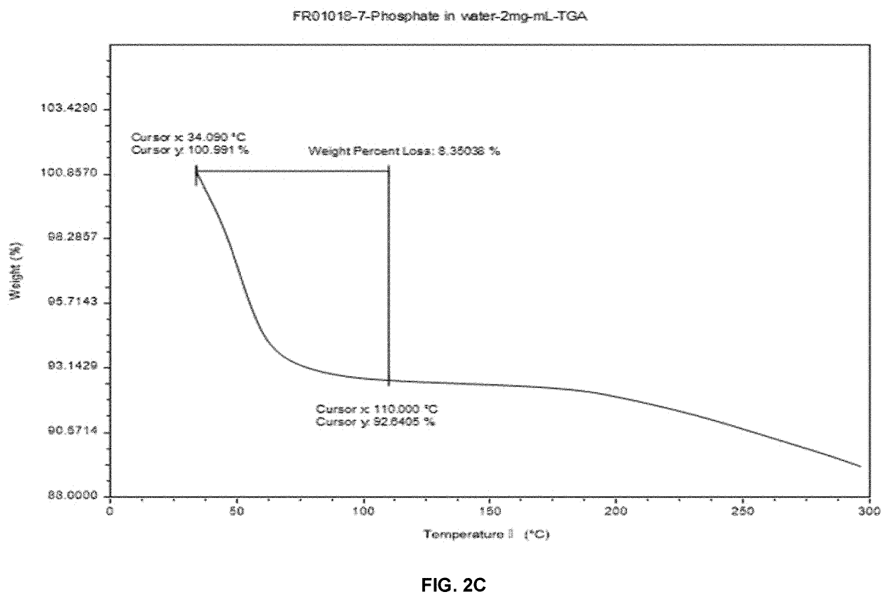

In embodiments, Form B is characterized by an XRPD pattern substantially similar to that shown in FIG. 2A.

In embodiments, Form B is characterized by an XRPD pattern comprising peaks shown in Table 4.

In embodiments, Form B is characterized by one, two, three, four, five, six, seven, eight, nine, ten, eleven, or more, XRPD peaks listed in Table 4.

In embodiments, Form B is characterized by at least one peak in a XRPD pattern selected from 12.7±0.2, 13.4±0.2, 13.6±0.2, or 39.1±0.2 °2θ, absent in Form A, described herein.

In embodiments, Form B is characterized by at least one peak in a XRPD pattern selected from 13.4±0.2, 13.6±0.2, or 39.1±0.2 °2θ, absent in Form C, described herein.

In embodiments, Form B is characterized by at least one peak in a XRPD pattern selected from 13.62±0.2 or 39.11±0.2 °2θ, absent in Form D, described herein.

In embodiments, Form B is characterized by at least one peak in a XRPD pattern selected from 12.6±0.2, 13.4±0.2, 13.6±0.2, or 39.1±0.2 °2θ, absent in Form E, described herein.

In embodiments, Form B is characterized by at least one peak in a XRPD pattern selected from 6.2±0.2, 13.4±0.2, or 39.11±0.2 °2θ, absent in Form F, described herein.

In embodiments, Form B is characterized by at least one peak in a XRPD pattern selected from 19.8±0.2 or 39.1±0.2 °2θ, absent in Form G, described herein.

In embodiments, Form B is characterized by at least one peak in a XRPD pattern selected from 6.2±0.2, 12.7±0.2, 13.4±0.2, 13.6±0.2, or 39.1±0.2 °2θ, absent in Form H, described herein.

In embodiments, Form B is characterized by at least one peak in a XRPD pattern selected from 6.2±0.2, 12.7±0.2, 13.4±0.2, 13.6±0.2, or 39.1±0.2 °2θ, absent in Form H, described herein.

In embodiments, Form B exhibits a DSC thermogram comprising a peak at 71±5, or 170±5° C., or combination thereof. In embodiments, the peak at 71±5° C. is associated with an enthalpy of 107±5 J/g and the peak at 170±5° C. is associated with an enthalpy of 2±5 J/g.

In embodiments, Form B exhibits a DSC thermogram comprising an endothermic peak with an onset at 33±5, or 165±5° C., or combination thereof.

In embodiments, Form B exhibits a DSC thermogram comprising a peak at 71±3, or 170±3° C., or combination thereof. In embodiments, the peak at 71±3° C. is associated with an enthalpy of 107±3 J/g and the peak at 170±3° C. is associated with an enthalpy of 2±3 Jg.

In embodiments, Form B exhibits a DSC thermogram comprising an endothermic peak with an onset at 33±3, or 165±3° C., or combination thereof.

In embodiments, Form B exhibits a DSC thermogram substantially similar to that shown in FIG. 2B.

In embodiment, Form B exhibits about a 5% to about 15% weight loss at a temperature of 110±5° C. as measured by TGA analysis. For example, Form B exhibits about a 5%, about 7%, about a 9%, about a 11%, about a 13%, or about a 15%, including all values therebetween, weight loss at a temperature of 110±5° C. as measured by TGA.

In embodiments, Form B exhibits an 8% weight loss at a temperature of 110±5° C. as measured by TGA analysis.

In embodiments, Form B exhibits a TGA thermogram substantially similar to that shown in FIG. 2C.

### II. Form C

In embodiments, the crystalline form of Compound I is Form C.

In embodiments, Form C is characterized by a peak in a XRPD pattern at 3.6±0.2 and/or 4.23.6±0.2 °2θ. In embodiments, the variance at any of these peaks is +0.1 °2θ.

In embodiments, Form C is characterized by peaks in an XRPD pattern at 16.8±0.2, 23.1±0.2, and 24.2±0.2 °2θ. In embodiments, the variance at any of these peaks is +0.1 °2θ.

In embodiments, Form C is characterized by peaks in an XRPD pattern at 16.8±0.2, 23.1±0.2, and 24.2±0.2 °2θ and at least one peak (e.g., 1, 2, 3, 4, or 5) an in XRPD pattern selected from 5.8±0.2, 11.5±0.2, 17.2±0.2, 17.5±0.2, and 18.4±0.2 °2θ. In embodiments, the variance at any of these peaks is +0.1 °2θ.

In embodiments, Form C is characterized by peaks in an XRPD pattern at 3.6±0.2, 4.2±0.2, 5.8±0.2, 7.1±0.2, 9.4±0.2, 11.5±0.2, 11.7±0.2, 12.8±0.2, 14.1±0.2, 16.8±0.2, 17.2±0.2, 17.5±0.2, 17.9±0.2, 18.2±0.2, 18.4±0.2, 19.0±0.2, 19.6±0.2, 20.4±0.2, 21.3±0.2, 22.1±0.2, 22.6±0.2, 23.1±0.2, 23.4±0.2, 24.2±0.2, 25.7±0.2, 26.2±0.2, 26.6±0.2, 28.5±0.2, 30.3±0.2, 31.0±0.2, 32.4±0.2, 34.0±0.2, and 36.2±0.2 °2θ. In embodiments, the variance at any of these peaks is ±0.1 °2θ.

In embodiments, Form C is characterized by peaks in an XRPD pattern at 16.8±0.1, 23.1±0.1, and 24.2±0.1 °2θ.

In embodiments, Form C is characterized by peaks in an XRPD pattern at 16.8±0.1, 23.1±0.1, and 24.2±0.1 °2θ and at least one peak (e.g., 1, 2, 3, 4, or 5) an in XRPD pattern selected from 5.8±0.1, 11.5±0.1, 17.2±0.1, 17.5±0.1, and 18.4±0.1 °2θ.

In embodiments, Form C is characterized by peaks in an XRPD pattern at 3.6±0.1, 4.2±0.1, 5.8±0.1, 7.1±0.1, 9.4±0.1, 11.5±0.1, 11.7±0.1, 12.8±0.1, 14.1±0.1, 16.8±0.1, 17.2±0.1, 17.5±0.1, 17.9±0.1, 18.2±0.1, 18.4±0.1, 19.0±0.1, 19.6±0.1, 20.4±0.1, 21.3±0.1, 22.1±0.1, 22.6±0.1, 23.1±0.1, 23.4±0.1, 24.2±0.1, 25.7±0.1, 26.2±0.1, 26.6±0.1, 28.5±0.1, 30.3±0.1, 31.0±0.1, 32.4±0.1, 34.0±0.1, and 36.2±0.1 °2θ.

In embodiments, Form C is characterized by at least one peak in a XRPD pattern selected from 3.6±0.2, 4.2±0.2, 12.8±0.2, 22.6±0.2, 30.3±0.2, 31.0±0.2, 32.4±0.2, or 33.5±0.2 020, absent in Form A, described herein.

In embodiments, Form C is characterized by at least one peak in a XRPD pattern selected from 3.6±0.2, 4.2±0.2, 7.1±0.2, 9.4±0.2, 11.5±0.2, 11.7±0.2, 12.8±0.2, 14.1±0.2, 16.8±0.2, 17.2±0.2, 17.5±0.2, 17.9±0.2, 18.2±0.2, 18.4±0.2, 20.4±0.2, 21.3±0.2, 22.1±0.2, 22.6±0.2, 24.2±0.2, 25.7±0.2, 28.5±0.2, 30.3±0.2, 31.0±0.2, 32.4±0.2, 33.5±0.2, or 36.2±0.2 °2θ, absent in Form B, described herein.

In embodiments, Form C is characterized by at least one peak in a XRPD pattern selected from 3.6±0.2, 4.2±0.2, 9.4±0.2, 14.1±0.2, 20.4±0.2, 22.1±0.2, 22.6±0.2, 28.5±0.2, 30.3±0.2, 31.0±0.2, 32.4±0.2, 33.5±0.2, or 36.3±0.2 °2θ, absent in Form D, described herein.

In embodiments, Form C is characterized by at least one peak in a XRPD pattern selected from 3.6±0.2, 4.2±0.2, 12.8±0.2, 30.3±0.2, 31.0±0.2, 32.4±0.2, or 36.2±0.2 °2θ, absent in Form E, described herein.

In embodiments, Form C is characterized by at least one peak in a XRPD pattern selected from 3.6±0.2, 4.2±0.2, 5.8±0.2, 7.1±0.2, 9.4±0.2, 17.5±0.2, 17.9±0.2, 18.2±0.2, 18.4±0.2, or 31.0±0.2, 33.5±0.2 °2θ, absent in Form F, described herein.

In embodiments, Form C is characterized by at least one peak in a XRPD pattern selected from 3.6±0.2, 4.2±0.2, 7.1±0.2, 9.4±0.2, 14.1±0.2, 16.8±0.2, 17.2±0.2, 17.5±0.2, 17.9±0.2, 18.2±0.2, 18.4±0.2, 22.1±0.2, 22.6±0.2, 24.2±0.2, 25.7±0.2, 28.5±0.2, 30.3±0.2, 32.4±0.2, or 33.5±0.2 °2θ, absent in Form G, described herein.

In embodiments, Form C is characterized by at least one peak in a XRPD pattern selected from 3.6±0.2, 4.2±0.2, 12.8±0.2, 26.6±0.2, 31.0±0.2, 32.4±0.2, 33.5±0.2, or 36.2±0.2 020, absent in Form H, described herein.

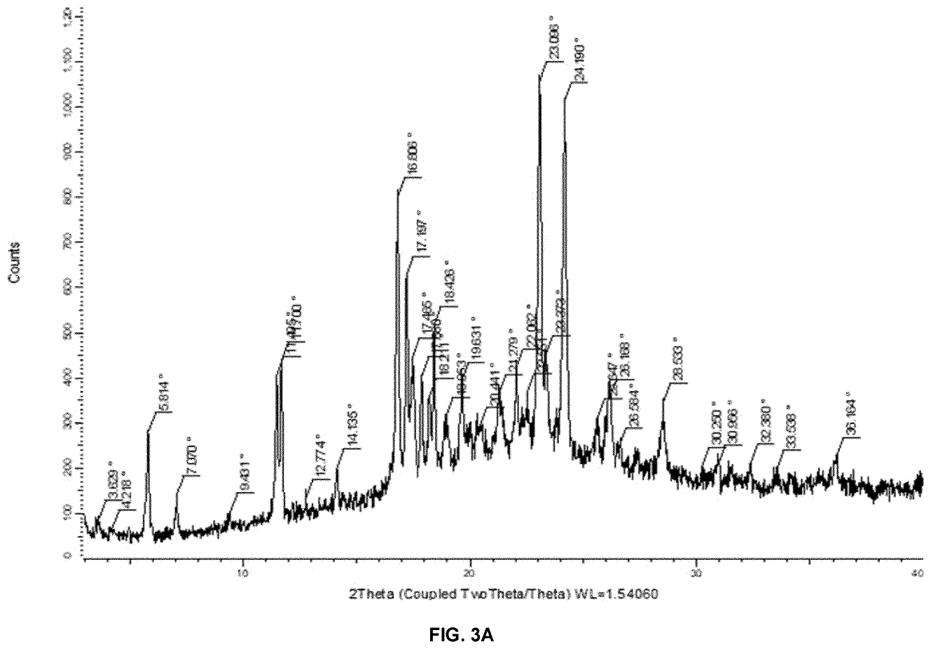

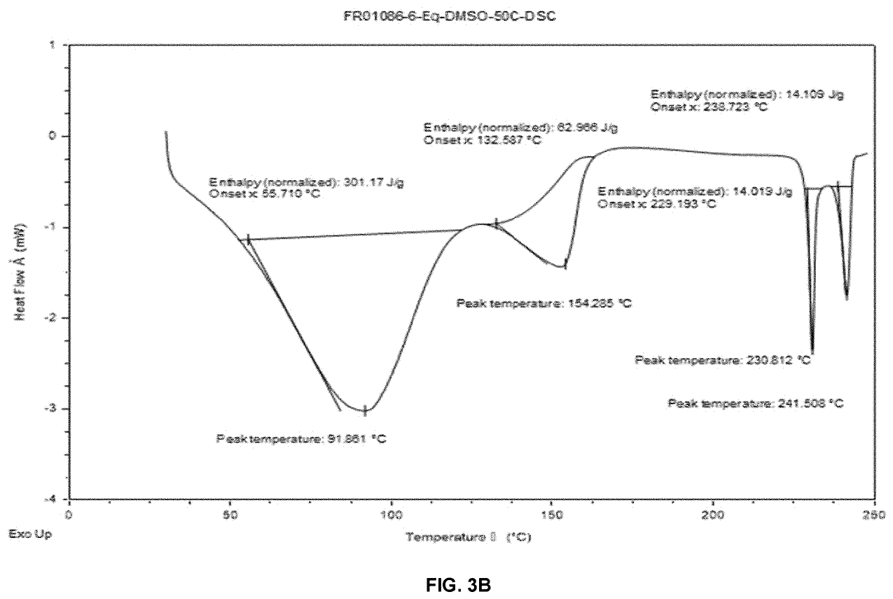

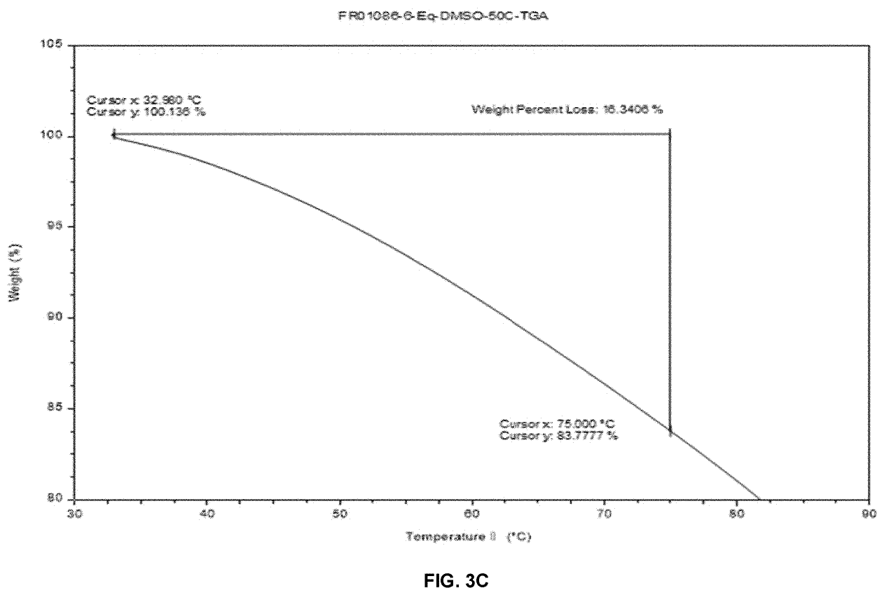

In embodiments, Form C is characterized by an XRPD pattern substantially similar to that shown in FIG. 3A.

In embodiments, Form C is characterized by an XRPD pattern comprising peaks shown in Table 5.

In embodiments, Form C is characterized by one, two, three, four, five, six, seven, eight, nine, ten, eleven, or more, XRPD peaks listed in Table 5.

In embodiments, Form C exhibits a DSC thermogram comprising a peak at 92±5, 154±5, 231±5, or 242±5° C., or any combination thereof. In embodiments, the peak at 92±5° C. is associated with an enthalpy of 301±5 J/g; the peak at 154±5° C. is associated with an enthalpy of 63±5 J/g; the peak at 231±5° C. is associated with an enthalpy of 14±5 J/g; and the peak at 242±5° C. is associated with an enthalpy of 14±5 J/g.

In embodiments, Form C is characterized by a DSC thermogram comprising an endothermic peak with an onset at 56±5, 133±5, 229±5, or 239±5° C., or any combination thereof.

In embodiments, Form C exhibits a DSC thermogram comprising a peak at 92±3, 154±3, 231±3, or 242±3° C., or any combination thereof. In embodiments, the peak at 92±3° C. is associated with an enthalpy of 301±5 J/g; the peak at 154±3° C. is associated with an enthalpy of 63±5 J/g; the peak at 231±3° C. is associated with an enthalpy of 14±5 J/g; and the peak at 242±3° C. is associated with an enthalpy of 14±5 J/g.

In embodiments, Form C is characterized by a DSC thermogram comprising an endothermic peak with an onset at 56±3, 133±3, 229±3, or 239±3° C., or any combination thereof.

In embodiments, Form C exhibits a DSC thermogram substantially similar to that shown in FIG. 3B.

In embodiments, Form C exhibits about a 10% to about 20% weight loss at a temperature of 75±5° C. as measured by TGA analysis. For example, Form C exhibits about a 10%, about 12%, about a 14%, about a 16%, about a 18%, or about a 20%, including all values therebetween, weight loss at a temperature of 75±5° C. as measured by TGA.

In embodiments, Form C exhibits a 16% weight loss at a temperature of 75±5° C. as measured by TGA analysis.

In embodiments, Form C exhibits a TGA thermogram substantially similar to that shown in FIG. 3C.

### IV. Form D

In embodiments, the crystalline form of Compound I is Form D.

In embodiments, Form D is characterized by peaks in an XRPD pattern at 5.8±0.2, 11.8±0.2, and 23.1±0.2 °2θ. In embodiments, the variance at any of these peaks is +0.1 °2θ.

In embodiments, Form D is characterized by peaks in an XRPD pattern at 5.8±0.2, 11.8±0.2, and 23.1±0.2 °2θ and at least one peak (e.g., 1, 2, 3, 4, or 5) in an XRPD pattern selected from 11.7±0.2, 12.5±0.2, 17.3±0.2, 23.4±0.2, and 24.1±0.2 °2θ. In embodiments, the variance at any of these peaks is +0.1 °2θ.

In embodiments, Form D is characterized by peaks in an XRPD pattern at 5.8±0.2, 6.0±0.2, 7.2±0.2, 8.9±0.2, 11.7±0.2, 11.8±0.2, 12.5±0.2, 13.0±0.2, 16.9±0.2, 17.3±0.2, 17.8±0.2, 18.5±0.2, 19.1±0.2, 19.7±0.2, 21.3±0.2, 23.1±0.2, 23.4±0.2, 24.1±0.2, 26.2±0.2, and 26.4±0.2 °2θ. In embodiments, the variance at any of these peaks is +0.1 °2θ.

In embodiments, Form D is characterized by peaks in an XRPD pattern at 5.8±0.1, 11.8±0.1, and 23.1±0.1 °2θ.

In embodiments, Form D is characterized by peaks in an XRPD pattern at 5.8±0.1, 11.8±0.1, and 23.1±0.1 °2θ and at least one peak (e.g., 1, 2, 3, 4, or 5) in an XRPD pattern selected from 11.7±0.1, 12.5±0.1, 17.3±0.1, 23.4±0.1, and 24.1±0.1 °2θ.

In embodiments, Form D is characterized by peaks in an XRPD pattern at 5.8±0.1, 6.0±0.1, 7.2±0.1, 8.9±0.1, 11.7±0.1, 11.8±0.1, 12.5±0.1, 13.0±0.1, 16.9±0.1, 17.3±0.1, 17.8±0.1, 18.5±0.1, 19.1±0.1, 19.7±0.1, 21.3±0.1, 23.1±0.1, 23.4±0.1, 24.1±0.1, 26.2±0.1, and 26.4±0.1 °2θ.

In embodiments, Form D is characterized by peaks in an XPRD pattern at 12.5±0.2 and 23.4±0.2 °2θ. In embodiments, Form D is further characterized by at least one peak in an XPRD pattern selected from 11.5±0.2, 21.3±0.2, and 26.4±0.2 °2θ. Form D is characterized by peaks in an XPRD pattern at 6.1±0.2, 8.9±0.2, 21.3±0.2, 23.4±0.2, and 26.4±0.2 °2θ.

In embodiments, Form D is characterized by at least one peak in a XRPD pattern selected from 12.5±0.2 and 13.0±0.2 °2θ, absent in Form A, described herein.

In embodiments, Form D is characterized by at least one peak in a XRPD pattern selected from 7.2±0.2, 8.9±0.2, 11.7±0.2, 11.8±0.2, 12.5±0.2, 16.9±0.2, 17.3±0.2, 17.8±0.2, 18.5±0.2, 19.1±0.2, 21.3±0.2, or 24.1±0.2 °2θ, absent in Form B, described herein.

In embodiments, Form D is characterized by a XRPD pattern at 8.9±0.2 °2θ, absent in Form C, described herein.

In embodiments, Form D is characterized by at least one peak in a XRPD pattern selected from 8.9±0.2, 12.5±0.2, or 13.0±0.2 °2θ, absent in Form E, described herein.

In embodiments, Form D is characterized by at least one peak in a XRPD pattern selected from 5.8±0.2, 6.0±0.2, 7.2±0.2, 8.9±0.2, 12.5±0.2, 13.0±0.2, 17.8±0.2, or 18.5±0.2 °2θ, absent in Form F, described herein.

In embodiments, Form D is characterized by at least one peak in a XRPD pattern selected from 7.2±0.2, 8.9±0.2, 16.9±0.2, 17.3±0.2, 17.8±0.2, 18.5±0.2, or 24.1±0.2 °2θ, absent in Form G, described herein.

In embodiments, Form D is characterized by at least one peak in a XRPD pattern selected from 12.5±0.2, 13.04±0.2, 26.2±0.2, or 26.4±0.2 °2θ, absent in Form H, described herein.

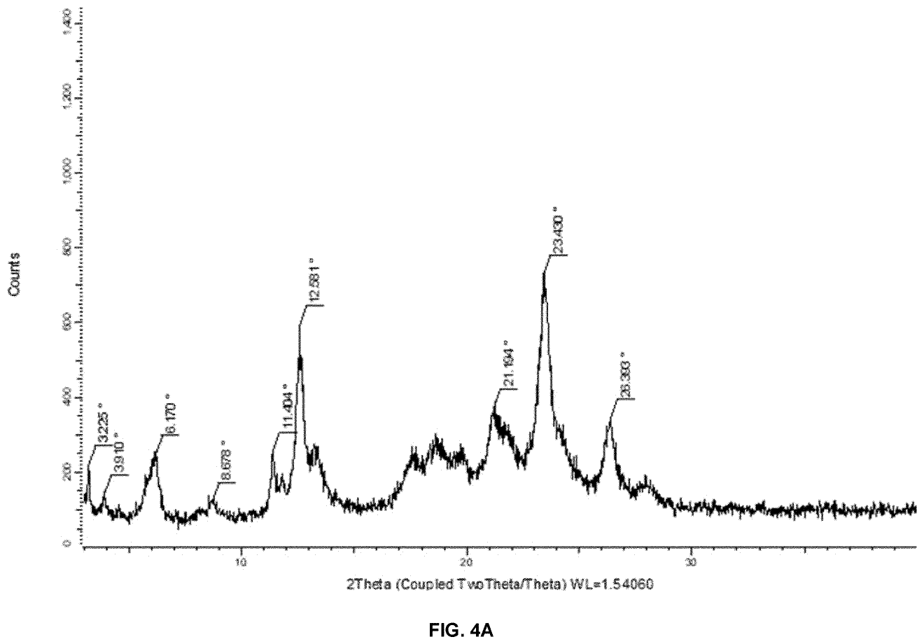

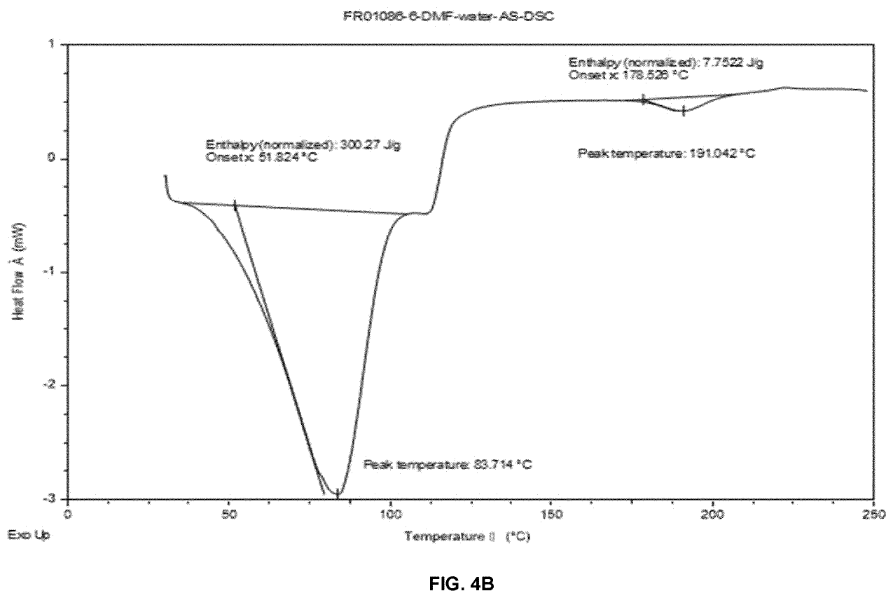

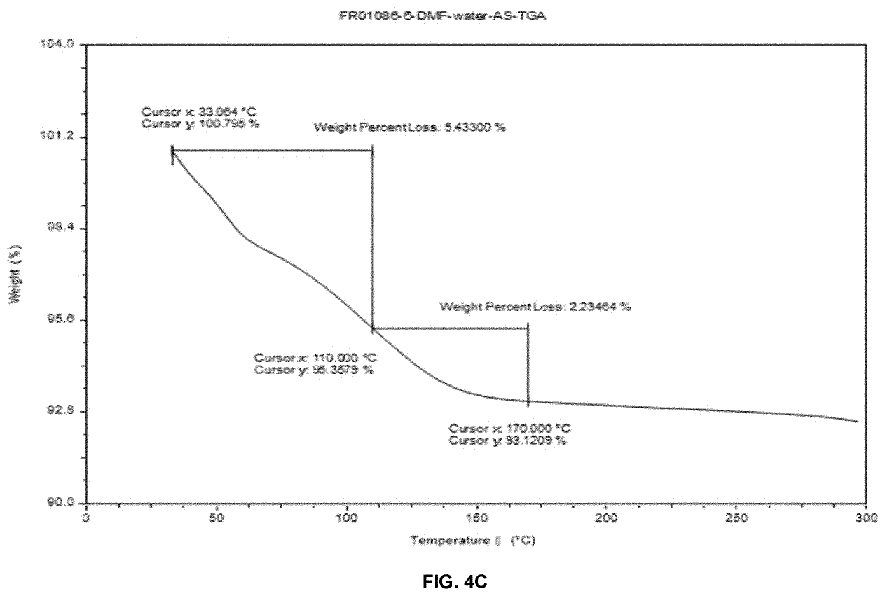

In embodiments, Form D is characterized by an XRPD pattern substantially similar to that shown in FIG. 4A.

In embodiments, Form D is characterized by an XRPD pattern comprising peaks shown in Table 6.

In embodiments, Form D is characterized by one, two, three, four, five, six, seven, eight, nine, ten, eleven, or more, XRPD peaks listed in Table 6.

In embodiments, Form D exhibits a DSC thermogram comprising a peak at 84±5, or 191±5° C., or combination thereof. In embodiments, the peak at 84±5° C. is associated with an enthalpy of 300±5 J/g and the peak at 191±5° C. is associated with an enthalpy of 8±5 J/g.

In embodiments, Form D exhibits a DSC thermogram comprising an endothermic peak with an onset at 52±5, or 179±5° C., or combination thereof.

In embodiments, Form D exhibits a DSC thermogram comprising a peak at 84±3, or 191±3° C., or combination thereof. In embodiments, the peak at 84±3° C. is associated with an enthalpy of 300±5 J/g and the peak at 191±3° C. is associated with an enthalpy of 8±5 J/g.

In embodiments, Form D exhibits a DSC thermogram comprising an endothermic peak with an onset at 52±3, or 179±3° C., or combination thereof.

In embodiments, Form D exhibits a DSC thermogram substantially similar to that shown in FIG. 4B.

In embodiments, Form D exhibits about a 1% to about a 10% weight loss at a temperature of 110±5° C. as measured by TGA analysis. For example, Form D exhibits about a 1%, about a 2%, about a 3%, about a 4%, about a 5%, about a 6%, about a 7%, about an 8%, about a 9%, or about a 10%, including all values therebetween, weight loss at a temperature of 110±5° C. as measured by TGA analysis.

In embodiments, Form D exhibits about a 0.1% to about 5% weight loss at a temperature of 170±5° C. as measured by TGA analysis. For example, Form D exhibits about a 0.1%, about a 1%, about a 2%, about a 3%, about a 4%, or about a 5%, including all values therebetween, weight loss at a temperature of 170±5° C. as measured by TGA analysis.

In some embodiments, Form D exhibits substantially no weight loss at a temperature of 170±5° C. as measured by TGA analysis.

In embodiments, Form D exhibits about a 1% to about 10% weight loss at a temperature of 110±5° C., about 0.1% to about 2% weight loss at 170±5° C. as measured by TGA analysis, or both.

In embodiments, Form D exhibits a 5% weight loss at a temperature of 110±5° C., a 2% weight loss at 170±5° C. as measured by TGA analysis, or both.

In embodiments, Form D exhibits a TGA thermogram substantially similar to that shown in FIG. 4C.

### V. Form E

In embodiments, the crystalline form of Compound I is Form E.

In embodiments, Form E is characterized by peaks in an XRPD pattern at 5.8±0.2, 17.4±0.2, and 22.9±0.2 °2θ. In embodiments, the variance at any of these peaks is +0.1 °2θ.

In embodiments, Form E is characterized by peaks in an XRPD pattern at 5.8±0.2, 17.4±0.2, and 22.9±0.2 °2θ and at least one peak (e.g., 1, 2, 3, 4, or 5) an in XRPD pattern selected from 11.6±0.2, 16.7±0.2, 17.1±0.2, 23.3±0.2, and 24.0±0.2 °2θ. In embodiments, the variance at any of these peaks is +0.1 °2θ.

In embodiments, Form E is characterized by peaks in an XRPD pattern at 5.8±0.2, 7.0±0.2, 9.4±0.2, 11.4±0.2, 11.6±0.2, 14.1±0.2, 16.7±0.2, 17.1±0.2, 17.4±0.2, 17.8±0.2, 18.1±0.2, 18.3±0.2, 18.8±0.2, 19.4±0.2, 20.2±0.2, 20.9±0.2, 21.1±0.2, 21.8±0.2, 22.1±0.2, 22.9±0.2, 23.3±0.2, 24.0±0.2, 25.4±0.2, 25.9±0.2, 26.3±0.2, 28.3±0.2, 33.3±0.2, and 35.7±0.2 °2θ. In embodiments, the variance at any of these peaks is +0.1 °2θ.

In embodiments, Form E is characterized by peaks in an XRPD pattern at 5.8±0.1, 17.4±0.1, and 22.9±0.1 °2θ.

In embodiments, Form E is characterized by peaks in an XRPD pattern at 5.8±0.1, 17.4±0.1, and 22.9±0.1 °2θ and at least one peak an in XRPD pattern selected from 11.6±0.1, 16.7±0.1, 17.1±0.1, 23.3±0.1, and 24.0±0.1 °2θ.

In embodiments, Form E is characterized by at least one peak in a XRPD pattern selected from 12.5±0.2 or 13.0±0.2 °2θ, absent in Form A, described herein.

In embodiments, Form E is characterized by at least one peak in a XRPD pattern selected from 7.0±0.2, 9.4±0.2, 11.4±0.2, 11.6±0.2, 14.1±0.2, 16.7±0.2, 17.1±0.2, 17.4±0.2, 17.8±0.2, 18.1±0.2, 18.3±0.2, 18.8±0.2, 19.4±0.2, 20.2±0.2, 20.9±0.2, 21.1±0.2, 21.8±0.2, 22.1±0.2, 22.9±0.2, 24.0±0.2, 25.4±0.2, 28.3±0.2, 33.3±0.2, or 35.7±0.2 °2θ, absent in Form B, described herein.

In embodiments, Form E is characterized by at least one peak in a XRPD pattern selected from 20.9±0.2 or 35.7±0.2 °2θ, absent in Form C, described herein.

In embodiments, Form E is characterized by at least one peak in a XRPD pattern selected from 9.4±0.2, 14.1±0.2, 20.2±0.2, 21.8±0.2, 22.1±0.2, 25.4±0.2, 28.3±0.2, 33.3±0.2, or 35.7±0.2 °2θ, absent in Form D, described herein.

In embodiments, Form E is characterized by at least one peak in a XRPD pattern selected from 5.8±0.2, 7.0±0.2, 9.4±0.2, 17.8±0.2, 18.1±0.2, 18.3±0.2, 18.8±0.2, or 33.3±0.2 °2θ, absent in Form F, described herein.

In embodiments, Form E is characterized by at least one peak in a XRPD pattern selected from 7.0±0.2, 9.4±0.2, 14.1±0.2, 17.1±0.2, 17.4±0.2, 17.8±0.2, 18.1±0.2, 18.3±0.2, 20.2±0.2, 21.8±0.2, 22.1±0.2, 22.9±0.2, 24.0±0.2, 25.4±0.2, 28.3±0.2, or 33.3±0.2 °2θ, absent in Form G, described herein.

In embodiments, Form E is characterized by at least one peak in a XRPD pattern selected from 33.3±0.2 or 35.7±0.2 °2θ, absent in Form H, described herein.

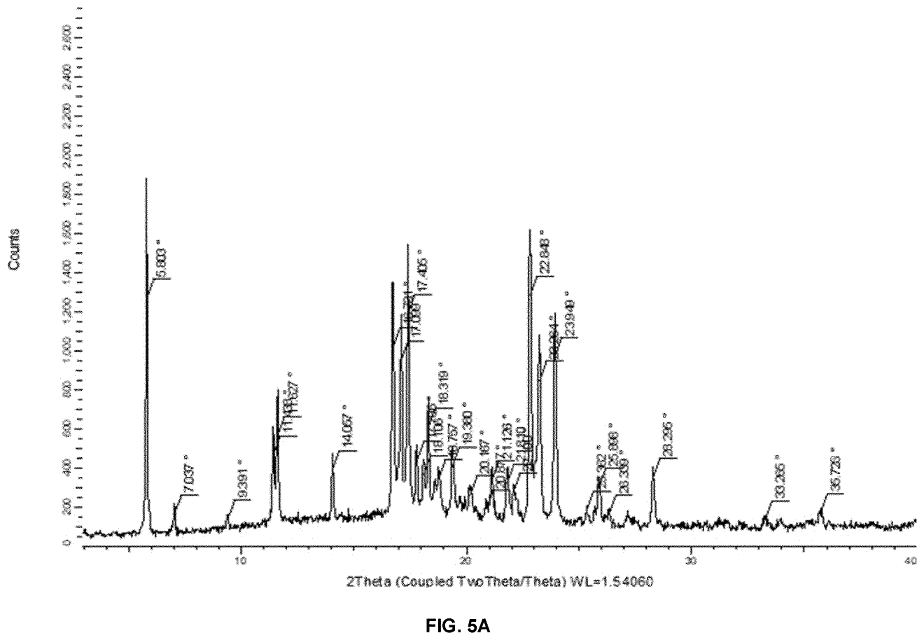

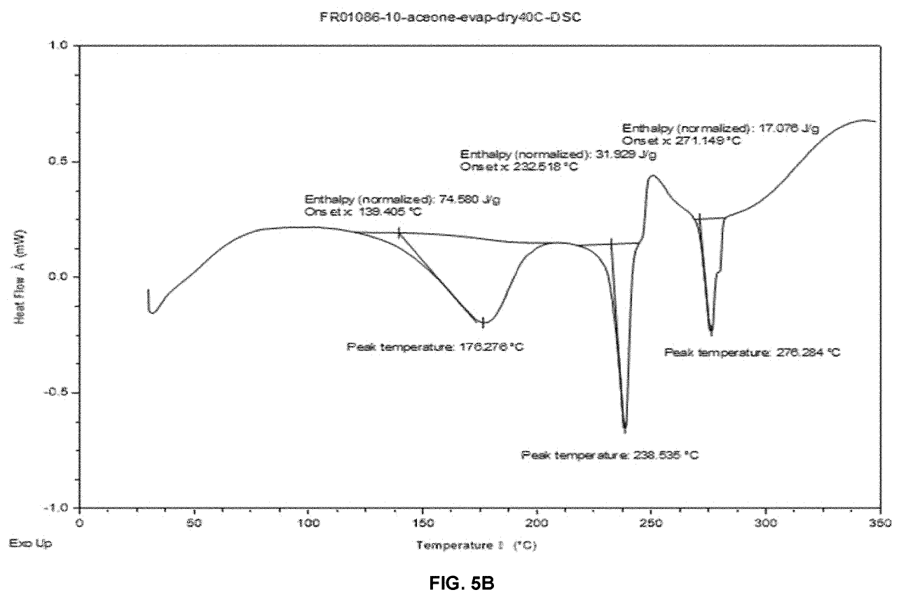

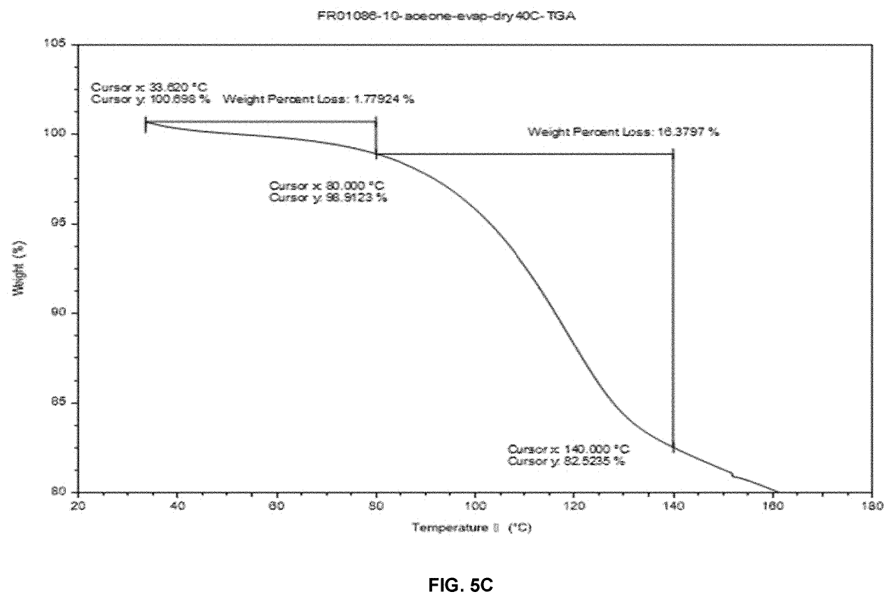

In embodiments, Form E is characterized by an XRPD pattern substantially similar to that shown in FIG. 5A.

In embodiments, Form E is characterized by an XRPD pattern comprising peaks shown in Table 7.

In embodiments, Form E is characterized by one, two, three, four, five, six, seven, eight, nine, ten, eleven, or more, XRPD peaks listed in Table 7.

In embodiments, Form E exhibits a DSC thermogram comprising a peak at 176±5, 239±5, or 276±5° C., or any combination thereof. In embodiments, the peak at 176±5° C. is associated with an enthalpy of 75±5 J/g; the peak at 239±5° C. is associated with an enthalpy of 32±5 J/g; and the peak at 276±5° C. is associated with an enthalpy of 17±5 J/g.

In embodiments, Form E exhibits a DSC thermogram comprising an endothermic peak with an onset at 139±5, 233±5, or 271±5° C., or any combination thereof.

In embodiments, Form E exhibits a DSC thermogram comprising a peak at 176±3, 239±3, or 276±3° C., or any combination thereof. In embodiments, the peak at 176±3° C. is associated with an enthalpy of 75±5 J/g; the peak at 239±3° C. is associated with an enthalpy of 32±5 J/g; and the peak at 276±3° C. is associated with an enthalpy of 17±5 J/g.

In embodiments, Form E exhibits a DSC thermogram comprising an endothermic peak with an onset at 139±3, 233±3, or 271±3° C., or any combination thereof.

In embodiments, Form E exhibits a DSC thermogram substantially similar to that shown in FIG. 5B.

In embodiments, Form E exhibits about exhibits about a 0.1% to about 5% weight loss at a temperature of 80±5° C. as measured by TGA analysis. For example, Form E exhibits about a 0.1%, about a 1%, about a 2%, about a 3%, about a 4%, or about a 5%, including all values therebetween, weight loss at a temperature of 80±5° C. as measured by TGA analysis.

In some embodiments, Form E exhibits substantially no weight loss at a temperature of 80±5° C. as measured by TGA analysis.

In embodiments, Form E exhibits about a 10% to about 20% weight loss at a temperature of 140±5° C. as measured by TGA analysis. For example, Form E exhibits about a 10%, about 12%, about a 14%, about a 16%, about a 18%, or about a 20%, including all values therebetween, weight loss at a temperature of 140±5° C. as measured by TGA.

In embodiments, Form E exhibits about a 0.1% to about a 5% weight loss at a temperature of 80±3° C., about a 10% to about 20% weight loss at 140±5° C. as measured by TGA analysis, or both.

In embodiments, Form E exhibits about a 2% weight loss at a temperature of 80±5° C., about a 16% weight loss at 140±5° C. as measured by TGA analysis, or both.

In embodiments, Form E exhibits a TGA thermogram substantially similar to that shown in FIG. 4C.

### VI. Form F

In embodiments, the crystalline form of Compound I is Form F.

In embodiments, Form F is characterized by at least one peak in a XRPD pattern selected from 15.8±0.2 or 29.4±0.2 °2θ. In embodiments, the variance at any of these peaks is +0.1 °2θ.

In embodiments, Form F is characterized by peaks in an XRPD pattern at 13.0±0.2, 17.0±0.2, and 19.3±0.2 °2θ. In embodiments, the variance at any of these peaks is +0.1 °2θ.

In embodiments, Form F is characterized by peaks in an XRPD pattern at 13.0±0.2, 17.0±0.2, and 19.3±0.2 °2θ and at least one peak (e.g., 1, 2, 3, 4, or 5) in an XRPD pattern selected from 20.0±0.2, 22.5±0.2, 22.6±0.2, 23.3±0.2, and 23.9±0.2 °2θ. In embodiments, the variance at any of these peaks is +0.1 °2θ.

In embodiments, Form F is characterized by peaks in an XRPD pattern at 11.8±0.2, 13.0±0.2, 13.9±0.2, 14.2±0.2, 14.5±0.2, 15.8±0.2, 16.4±0.2, 17.0±0.2, 19.3±0.2, 20.0±0.2, 21.0±0.2, 21.8±0.2, 22.1±0.2, 22.5±0.2, 22.6±0.2, 23.3±0.2, 23.7±0.2, 23.9±0.2, 24.1±0.2, 25.3±0.2, 25.5±0.2, 26.3±0.2, 26.8±0.2, 27.2±0.2, 27.9±0.2, 28.3±0.2, 29.4±0.2, 30.4±0.2, 31.7±0.2, 31.8±0.2, 32.2±0.2, 32.6±0.2, 35.6±0.2, 35.9±0.2, and 39.5±0.2 °2θ. In embodiments, the variance at any of these peaks is +0.1 °2θ.

In embodiments, Form F is characterized by peaks in an XRPD pattern at 13.0±0.1, 17.0±0.1, and 19.3±0.1 °2θ.

In embodiments, Form F is characterized by peaks in an XRPD pattern at 13.0±0.1, 17.0±0.1, and 19.3±0.1 °2θ and at least one peak (e.g., 1, 2, 3, 4, or 5) in an XRPD pattern selected from 20.0±0.1, 22.5±0.1, 22.6±0.1, 23.3±0.1, and 23.9±0.1 °2θ.

In embodiments, Form F is characterized by peaks in an XRPD pattern at 11.8±0.1, 13.0±0.1, 13.9±0.1, 14.2±0.1, 14.5±0.1, 15.8±0.1, 16.4±0.1, 17.0±0.1, 19.3±0.1, 20.0±0.1, 21.0±0.1, 21.8±0.1, 22.1±0.1, 22.5±0.1, 22.6±0.1, 23.3±0.1, 23.7±0.1, 23.9±0.1, 24.1±0.1, 25.3±0.1, 25.5±0.1, 26.3±0.1, 26.8±0.1, 27.2±0.1, 27.9±0.1, 28.3±0.1, 29.4±0.1, 30.4±0.1, 31.7±0.1, 31.8±0.1, 32.2±0.1, 32.6±0.1, 35.6±0.1, 35.9±0.1, and 39.5±0.1 °2θ.

In embodiments, Form F is characterized by at least one peak in a XRPD pattern selected from 13.0±0.2, 15.8±0.2, 22.6±0.2, 23.7±0.2, 29.4±0.2, 30.4±0.2, 31.7±0.2, 31.8±0.2, 32.2±0.2, 32.6±0.2, or 39.5±0.2 °2θ, absent in Form A, described herein.

In embodiments, Form F is characterized by at least one peak in a XRPD pattern selected from 11.8±0.2, 14.2±0.2, 14.5±0.2, 15.8±0.2, 16.4±0.2, 17.0±0.2, 19.3±0.2, 21.0±0.2, 21.8±0.2, 22.1±0.2, 22.5±0.2, 22.6±0.2, 23.9±0.2, 24.1±0.2, 25.3±0.2, 25.5±0.2, 26.8±0.2, 27.2±0.2, 27.9±0.2, 28.3±0.2, 29.4±0.2, 30.4±0.2, 31.7±0.2, 31.8±0.2, 32.2±0.2, 32.6±0.2, 35.6±0.2, 35.9±0.2 °2θ, absent in Form B, described herein.

In embodiments, Form F is characterized by peaks in an XRPD pattern at 15.8±0.2, 27.2±0.2, 27.9±0.2, 29.4±0.2, 31.7±0.2, 31.8, 35.6±0.2, or 38.5±0.2 °2θ, absent in Form C, described herein.

In embodiments, Form F is characterized by peaks in an XRPD pattern at 13.9±0.2, 14.2±0.2, 14.5±0.2, 15.8±0.2, 16.4±0.2, 21.8±0.2, 22.5±0.2, 22.6±0.2, 25.3±0.2, 25.5±0.2, 26.8±0.2, 27.2±0.2, 27.9±0.2, 28.3±0.2, 29.4±0.2, 30.4±0.2, 31.7±0.2, 31.8±0.2, 32.2±0.2, 32.6±0.2, 35.6±0.2, 35.9±0.2, or 39.5±0.2 °2θ, absent in Form D, described herein.

In embodiments, Form F is characterized by peaks in an XRPD pattern at 13.0±0.2, 15.8±0.2, 29.4±0.2, 30.4±0.2, 31.7±0.2, 31.8±0.2, 32.2±0.2, 32.6±0.2, or 39.5±0.2 °2θ, absent in Form E, described herein.

In embodiments, Form F is characterized by peaks in an XRPD pattern at 13.9±0.2, 14.2±0.2, 14.5±0.2, 15.8±0.2, 16.4±0.2, 17.0±0.2, 20.0±0.2, 21.8±0.2, 22.5±0.2, 22.6±0.2, 23.9±0.2, 24.1±0.2, 25.3±0.2, 25.5±0.2, 27.2±0.2, 29.4±0.2, 30.4±0.2, 32.2±0.2, 32.6±0.2, 35.6±0.2, or 39.5±0.2 °2θ, absent in Form G, described herein.

In embodiments, Form F is characterized by peaks in an XRPD pattern at 13.0±0.2, 15.8±0.2, 22.5±0.2, 23.25±0.2, 23.7±0.2, 29.4±0.2, 31.7±0.2, 31.8±0.2, 32.2±0.2, 32.6±0.2, 35.6±0.2, 35.9±0.2, or 39.5±0.2 °2θ, absent in Form H, described herein.

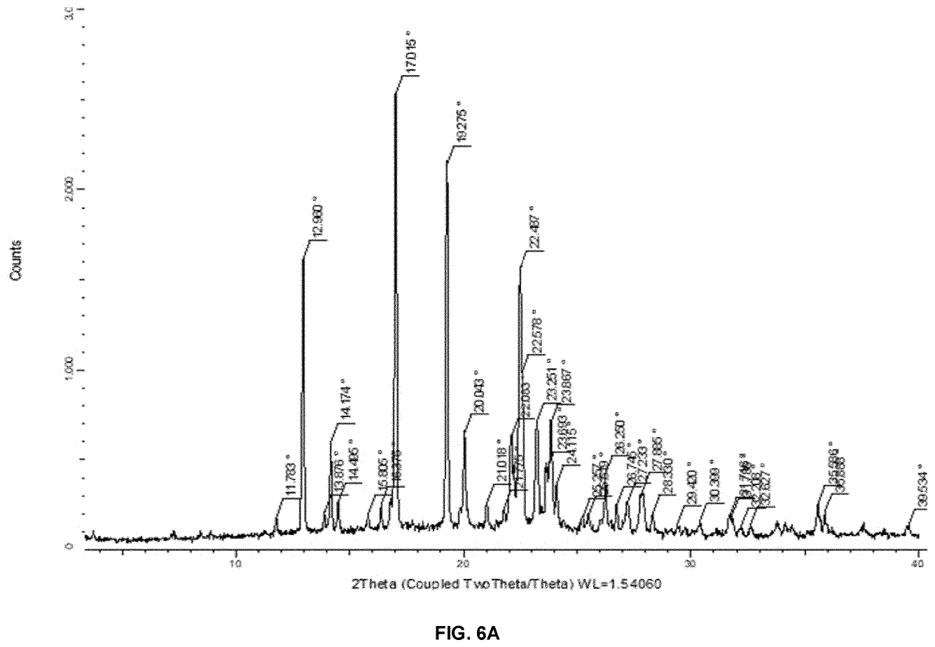

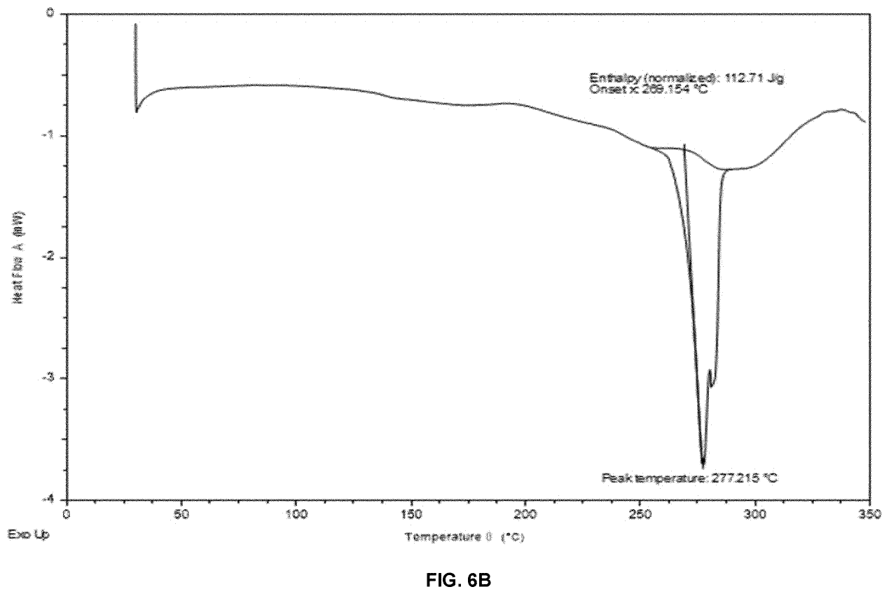

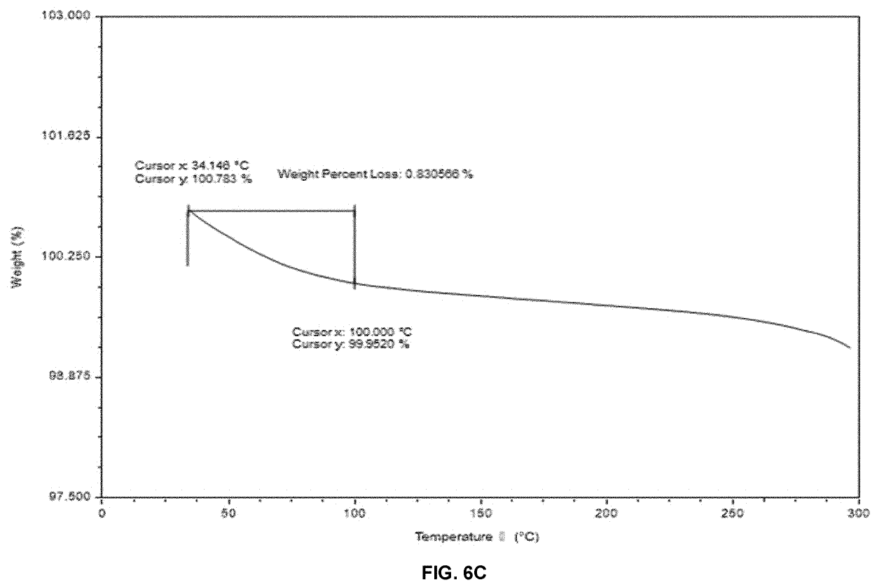

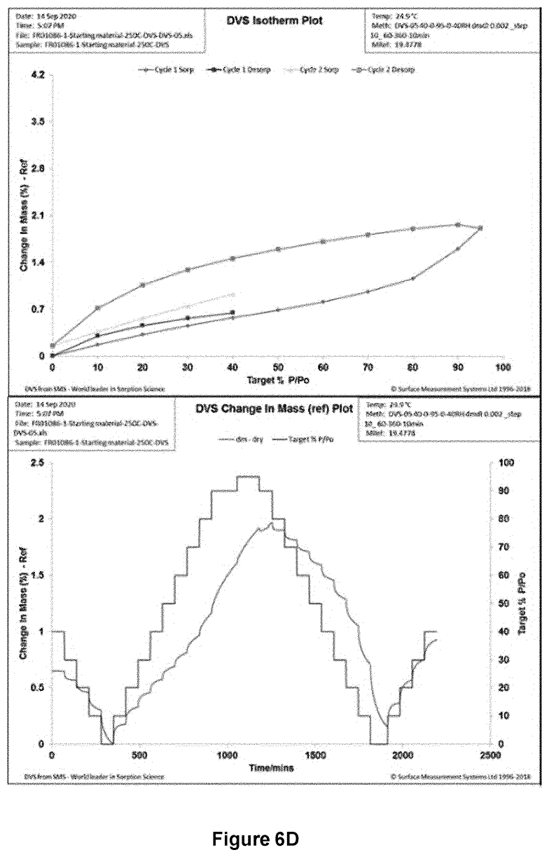

In embodiments, Form F is characterized by an XRPD pattern substantially similar to that shown in FIG. 6A.

In embodiments, Form F is characterized by an XRPD pattern comprising peaks shown in Table 8.

In embodiments, Form F is characterized one, two, three, four, five, six, seven, eight, nine, ten, eleven, or more, XRPD peaks listed in Table 8.

In embodiments, Form F exhibits a DSC thermogram comprising a peak at 277±5° C. In embodiments, the peak at 277±5° C. is associated with an enthalpy of 113±5 J/g.

In embodiments, Form F exhibits a DSC thermogram comprising an endothermic peak with an onset at 269±5° C.

In embodiments, Form F exhibits a DSC thermogram comprising a peak at 277±3° C. In embodiments, the peak at 277±3° C. is associated with an enthalpy of 113±5 J/g.

In embodiments, Form E exhibits a DSC thermogram comprising an endothermic peak with an onset at 269±3° C.

In embodiments, Form F exhibits a DSC thermogram substantially similar to that shown in FIG. 6B.

In embodiments, Form F exhibits a weight loss of less than about 1% at a temperature of 100±5° C. as measured by TGA. For example, Form F exhibits a weight loss of less than about 1%, less than about 0.1%, less than about 0.01%, or less than about 0.001% as measured by TGA.

In embodiments, Form F exhibits substantially no weight loss at a temperature of 100±5° C. as measured by TGA.

In embodiments, Form F exhibits a TGA thermogram substantially similar to that shown in FIG. 6C.

### VI. Form G

In embodiments, the crystalline form of Compound I is Form G.

In embodiments, Form G is characterized by peaks in an XRPD pattern at 12.6±0.2, 21.2±0.2, and 23.5±0.2 °2θ. In embodiments, the variance at any of these peaks is +0.1 °2θ.

In embodiments, Form G is characterized by peaks in an XRPD pattern at 12.6±0.2, 21.2±0.2, and 23.5±0.2 °2θ and at least one peak (e.g., 1, 2, 3, 4, or 5) in an XRPD pattern selected from 6.2±0.2, 13.4±0.2, 19.0±0.2, 26.2±0.2, and 26.5±0.2 °2θ. In embodiments, the variance at any of these peaks is +0.1 °2θ.

In embodiments, Form G is characterized by peaks in an XRPD pattern at 6.2±0.2, 11.4±0.2, 12.6±0.2, 13.4±0.2, 19.0±0.2, 21.2±0.2, 23.5±0.2, 26.2±0.2, 26.5±0.2, 28.0±0.2, 31.4±0.2, and 36.0±0.2 °2θ. In embodiments, the variance at any of these peaks is +0.1 °2θ.

In embodiments, Form G is characterized by peaks in an XRPD pattern at 12.6±0.1, 21.2±0.1, and 23.5±0.1 °2θ.

In embodiments, Form G is characterized by peaks in an XRPD pattern at 12.6±0.1, 21.2±0.1, and 23.5±0.1 °2θ and at least one peak (e.g., 1, 2, 3, 4, or 5) in an XRPD pattern selected from 6.2±0.1, 13.4±0.1, 19.0±0.1, 26.2±0.1, and 26.5±0.1 °2θ.

In embodiments, Form G is characterized by peaks in an XRPD pattern at 6.2±0.1, 11.4±0.1, 12.6±0.1, 13.4±0.1, 19.0±0.1, 21.2±0.1, 23.5±0.1, 26.2±0.1, 26.5±0.1, 28.0±0.1, 31.4±0.1, and 36.0±0.1 °2θ.

In embodiments, Form G is characterized by at least one peak in a XRPD pattern selected from 12.6±0.2, 13.4±0.2, or 31.4±0.2 °2θ, absent in Form A, described herein.

In embodiments, Form G is characterized by at least one peak in a XRPD pattern selected from 11.4±0.2, 21.2±0.2, 23.5±0.2, 28.0±0.2, 31.4±0.2, or 36.0±0.2 °2θ, absent in Form B, described herein.

In embodiments, Form G is characterized by at least one peak in a XRPD pattern selected from 13.4±0.2 or 28.0±0.2 °2θ, absent in Form C, described herein.

In embodiments, Form G is characterized by at least one peak in a XRPD pattern selected from 28.0±0.2, 31.4±0.2, or 36.0±0.2 °2θ, absent in Form D, described herein.

In embodiments, Form G is characterized by at least one peak in a XRPD pattern selected from 12.6±0.2, 13.4±0.2, or 31.4±0.2 °2θ, absent in Form E, described herein.

In embodiments, Form G is characterized by at least one peak in a XRPD pattern selected from 13.4±0.2 or 19.0±0.2 °2θ, absent in Form E, described herein.

In embodiments, Form G is characterized by at least one peak in a XRPD pattern selected from 12.6±0.2, 13.4±0.2, 23.5±0.2, 26.5±0.2, 31.4±0.2, or 36.0±0.2 °2θ, absent in Form H, described herein.

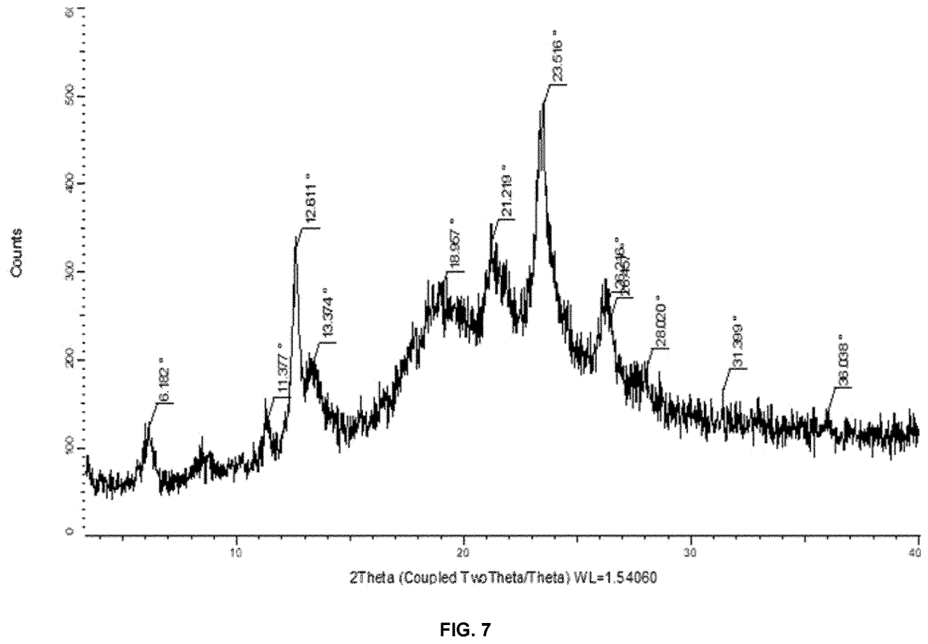

In embodiments, Form G is characterized by an XRPD pattern substantially similar to that shown in FIG. 7A.

In embodiments, Form G is characterized by an XRPD pattern comprising peaks shown in Table 10.

In embodiments, Form G is characterized by one, two, three, four, five, six, seven, eight, nine, ten, eleven, or more, XRPD peaks listed in Table 10.

### VIII. Form H

In embodiments, the crystalline form of Compound I is Form H.

In embodiments, Form H is characterized by a peak in a XRPD pattern 30.3±0.2 °2θ, absent in Form A, described herein. In embodiments, the variance at any of these peaks is +0.1 °2θ.

In embodiments, Form H is characterized by peaks in an XRPD pattern at 5.7±0.2, 6.0±0.2, and 11.7±0.2 °2θ. In embodiments, the variance at any of these peaks is +0.1 °2θ.

In embodiments, Form H is characterized by peaks in an XRPD pattern at 5.7±0.2, 6.0±0.2, and 11.7±0.2 °2θ and at least one peak (e.g., 1, 2, 3, 4, or 5) in an XPRD pattern selected from 7.1±0.2, 11.4±0.2, 17.2±0.2, 23.0±0.2, and 24.0±0.2 °2θ. In embodiments, the variance at any of these peaks is +0.1 °2θ.

In embodiments, Form H is characterized by peaks in an XPRD pattern at 5.7±0.2, 6.0±0.2, 7.1±0.2, 9.2±0.2, 11.4±0.2, 11.7±0.2, 14.1±0.2, 16.5±0.2, 17.2±0.2, 17.8±0.2, 18.4±0.2, 19.1±0.2, 19.7±0.2, 20.1±0.2, 20.6±0.2, 21.4±0.2, 22.0±0.2, 23.0±0.2, 24.0±0.2, 25.4±0.2, 26.0±0.2, 27.1±0.2, 28.2±0.2, and 30.3±0.2 °2θ. In embodiments, the variance at any of these peaks is +0.1 °2θ.

In embodiments, Form H is characterized by peaks in an XRPD pattern at 5.7±0.1, 6.0±0.1, and 11.7±0.1 °2θ.

In embodiments, Form H is characterized by peaks in an XRPD pattern at 5.7±0.1, 6.0±0.1, and 11.7±0.1 °2θ and at least one peak (e.g., 1, 2, 3, 4, or 5) in an XPRD pattern selected from 7.1±0.1, 11.4±0.1, 17.2±0.1, 23.0±0.1, and 24.0±0.1 °2θ.

In embodiments, Form H is characterized by peaks in an XPRD pattern at 5.7±0.1, 6.0±0.1, 7.1±0.1, 9.2±0.1, 11.4±0.1, 11.7±0.1, 14.1±0.1, 16.5±0.1, 17.2±0.1, 17.8±0.1, 18.4±0.1, 19.1±0.1, 19.7±0.1, 20.1±0.1, 20.6±0.1, 21.4±0.1, 22.0±0.1, 23.0±0.1, 24.0±0.1, 25.4±0.1, 26.0±0.1, 27.1±0.1, 28.2±0.1, and 30.3±0.1 °2θ.

In embodiments, Form H is characterized by at least one peak in a XRPD pattern selected from 9.2±0.2, 11.4±0.2, 11.7±0.2, 14.1±0.2, 16.5±0.2, 17.2±0.2, 17.8±0.2, 18.4±0.2, 20.1±0.2, 20.6±0.2, 21.4±0.2, 22.0±0.2, 24.0±0.2, 25.4±0.2, 27.1±0.2, 28.2±0.2, or 30.3±0.2 °2θ, absent in Form B, described herein.

In embodiments, Form H is characterized by a peak in a XRPD pattern at 27.1±0.2 020, absent in Form C, described herein.

In embodiments, Form H is characterized by at least one peak in a XRPD pattern selected from 14.1±0.2, 20.1±0.2, 20.6±0.2, 22.0±0.2, 25.4±0.2, 27.1±0.2, 28.2±0.2, or 30.3±0.2 020, absent in Form D, described herein.

In embodiments, Form H is characterized by at least one peak in a XRPD pattern selected from 27.1±0.2 or 30.3±0.2 °2θ, absent in Form E, described herein.

In embodiments, Form H is characterized by at least one peak in a XRPD pattern selected from 17.8±0.2, 18.4±0.2, or 20.57±0.2 °2θ, absent in Form F, described herein.

In embodiments, Form H is characterized by at least one peak in a XRPD pattern selected from 7.1±0.2, 9.2±0.2, 14.1±0.2, 16.5±0.2, 17.2±0.2, 17.8±0.2, 18.4±0.2, 19.7±0.2, 20.1±0.2, 20.6±0.2, 22.0±0.2, 23.0±0.2, 24.0±0.2, 25.4±0.2, 27.1±0.2, or 30.3±0.2 °2θ, absent in Form G, described herein.

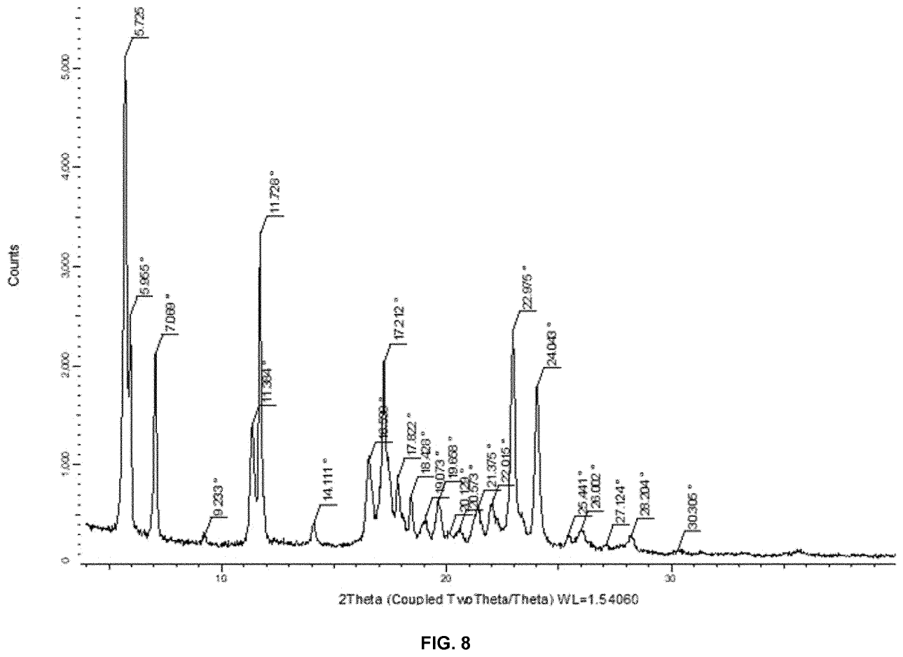

In embodiments, Form H is characterized by an XRPD pattern substantially similar to that shown in FIG. 8A.

In embodiments, Form H is characterized by an XRPD pattern comprising peaks shown in Table 11.

In embodiments, Form H is characterized by one, two, three, four, five, six, seven, eight, nine, ten, eleven, or more, XRPD peaks listed in Table 11.

### IX Form I

In embodiments, the crystalline form of Compound I is Form I.

In embodiments, Form I is characterized by at least one peak in a XRPD pattern at 6.3±0.2 °2θ. In embodiments, the variance at this peak is +0.1 °2θ.

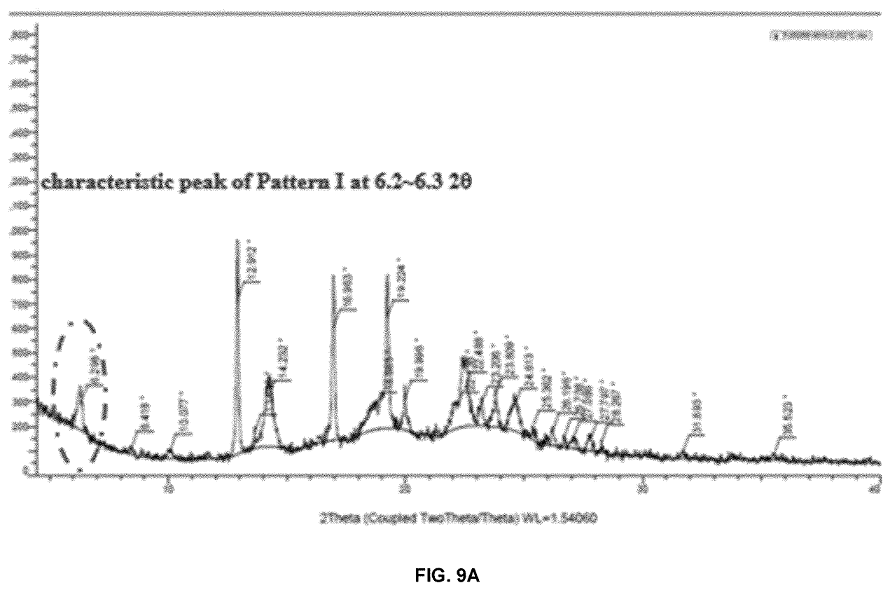

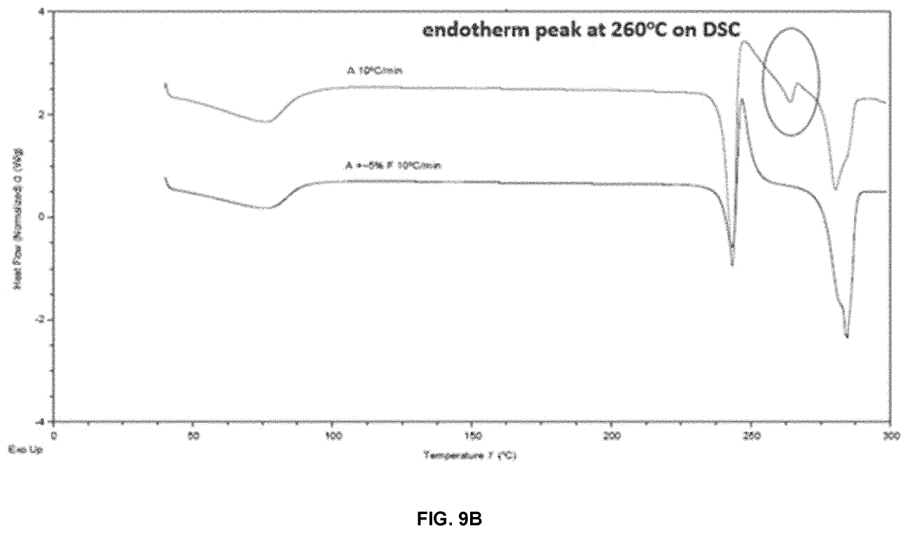

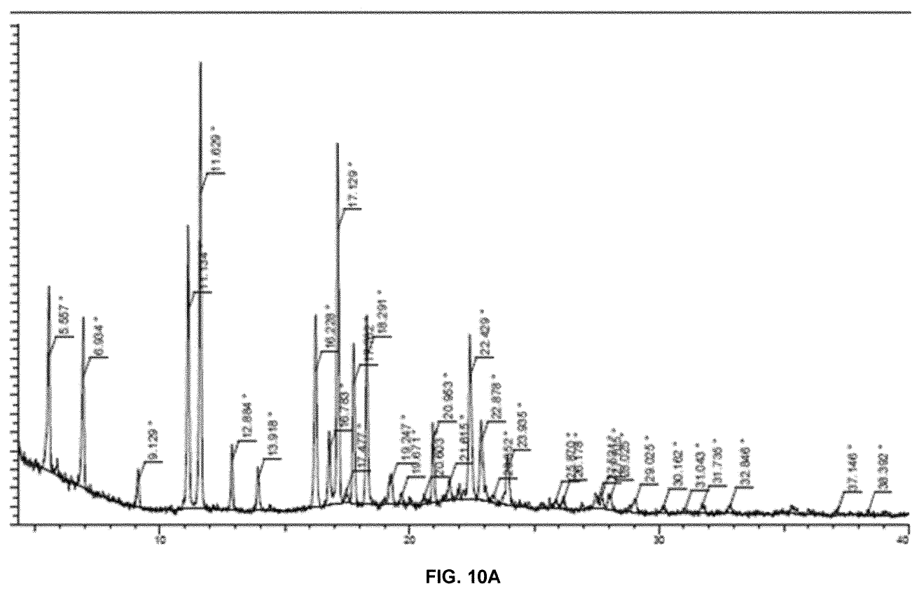

In embodiments, Form I is characterized by an XRPD pattern substantially similar to that shown in FIG. 9A.

In embodiments, Form I exhibits a DSC thermogram comprising at peak at 260±5° C.

In embodiments, Form I exhibits a DSC thermogram substantially similar to that shown in FIG. 9B.

### X. Form J

In embodiments, the crystalline form of Compound I is Form J.

In embodiments, Form J is characterized by peaks in an XRPD pattern at 11.1±0.2, 11.6±0.2, and 17.1±0.2 °2θ. In embodiments, the variance at any of these peaks is +0.1 °2θ.

In embodiments, Form J is characterized by peaks in an XPRD pattern at 5.6±0.2, 6.9±0.2, 9.1±0.2, 11.1±0.2, 11.6±0.2, 12.9±0.2, 13.9±0.2, 16.2±0.2, 16.8±0.2, 17.1±0.2, 17.5±0.2, 17.8±0.2, 18.29±0.2, 19.2±0.2, 19.7±0.2, 20.6±0.2, 21.0±0.2, 21.6±0.2, 22.4±0.2, 22.9±0.2, 23.4±0.2, 23.9±0.2, 25.9±0.2, 26.2±0.2, 27.5±0.2, 27.7±0.2, 28.0±0.2, 29.0±0.2, 30.1±0.2, 31.0±0.2, 31.7±0.2, 32.9±0.2, 37.2±0.2, and 38.4±0.2 °2θ. In embodiments, the variance at any of these peaks is +0.1 °2θ.

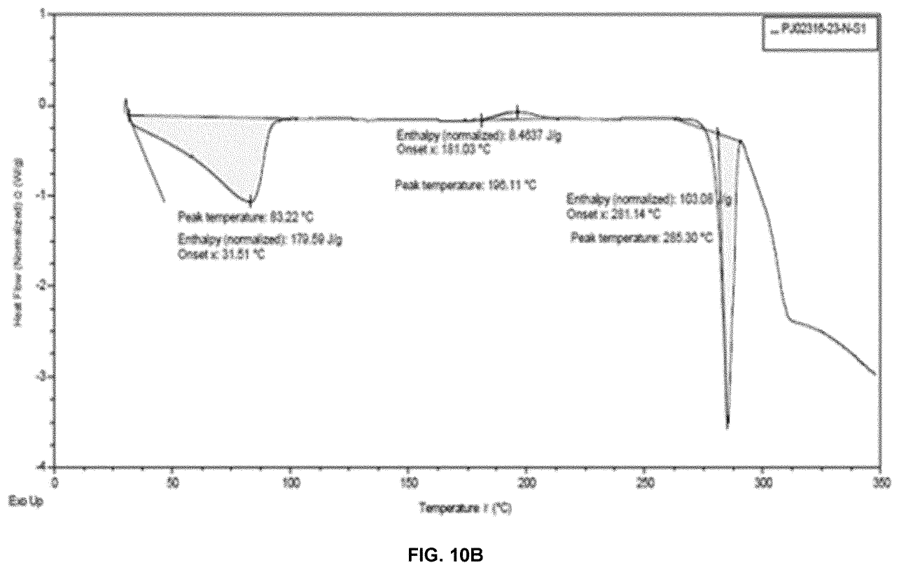

In embodiments, Form J is characterized by an XRPD pattern substantially similar to that shown in FIG. 10A.

In embodiments, Form J is characterized by an XRPD pattern comprising peaks shown in Table 13.

In embodiments, Form J is characterized one, two, three, four, five, six, seven, eight, nine, ten, eleven, or more, XRPD peaks listed in Table 13.

In embodiments, Form J exhibits a DSC thermogram comprising a peak at exhibits a DSC thermogram comprising a peak at 83±5° C., 196±5° C., and 285±5° C.

In embodiments, Form J exhibits a DSC thermogram substantially similar to that shown in FIG. 10B.

### XI. Form K

In embodiments, the crystalline form of Compound I is Form K.

In embodiments, Form K is characterized by peaks in an XRPD pattern at 5.4±0.2, 13.4±0.2, and 17.9±0.2 °2θ. In embodiments, the variance at any of these peaks is +0.1 °2θ.

In embodiments, Form K is characterized by peaks in an XRPD pattern at 5.5±0.2, 7.3±0.2, 11.4±0.2, 12.0±0.2, 12.9±0.2, 13.4±0.2, 14.6±0.2, 16.1±0.2, 16.9±0.2, 17.4±0.2, 17.9±0.2, 18.4±0.2, 19.0±0.2, 19.9±0.2, 21.1±0.2, 21.5±0.2, 21.7±0.2, 21.9±0.2, 22.2±0.2, 22.9±0.2, 23.2±0.2, 24.1±0.2, 24.8±0.2, 25.1±0.2, 27.8±0.2, 26.3±0.2, 26.7±0.2, 27.3±0.2, 27.6±0.2, 28.5±0.2, 29.7±0.2, 32.5±0.2, 33.9±0.2, 34.7±0.2, 35.3±0.2, 37.3±0.2, and 38.3±0.2 °2θ. In embodiments, the variance at any of these peaks is +0.1 °2θ.

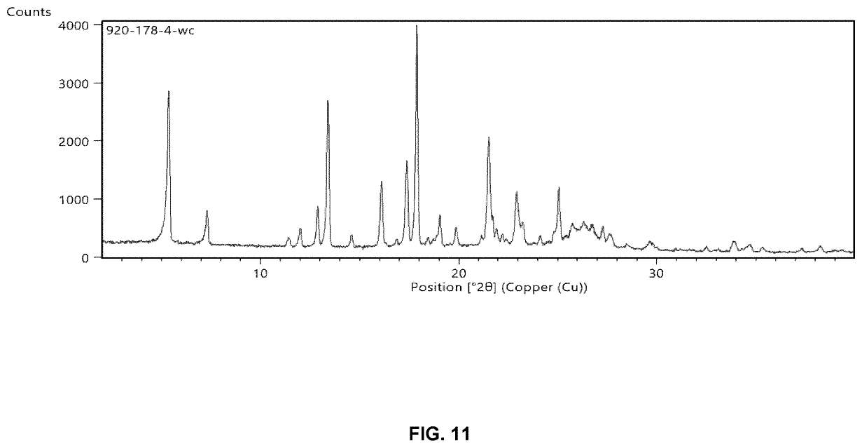

In embodiments, Form K is characterized by an XRPD pattern substantially similar to that shown in FIG. 11.

In embodiments, Form K is characterized by an XRPD pattern comprising peaks shown in Table 14.

In embodiments, Form K is characterized one, two, three, four, five, six, seven, eight, nine, ten, eleven, or more, XRPD peaks listed in Table 14.

### XII. Form L

In embodiments, the crystalline form of Compound I is Form L.

In embodiments, Form L is characterized by peaks in an XRPD pattern at 4.3±0.2, 8.2±0.2, and 8.6±0.2 °2θ. In embodiments, the variance at any of these peaks is +0.1 °2θ.

In embodiments, Form L is characterized by peaks in an XRPD pattern at 4.3±0.2, 8.2±0.2, 8.6±0.2, 12.9±0.2, 13.4±0.2, 15.4±0.2, 15.9±0.2, 17.2±0.2, 17.7±0.2, 18.2±0.2, 18.6±0.2, 19.2±0.2, 20.5±0.2, 21.5±0.2, 22.2±0.2, 24.1±0.2, 24.9±0.2, 25.7±0.2, 26.0±0.2, 26.6±0.2, 27.1±0.2, 27.5±0.2, 28.1±0.2, 29.2±0.2, 30.2±0.2, 31.2±0.2, and 38.5±0.2 °2θ. In embodiments, the variance at any of these peaks is +0.1 °2θ.

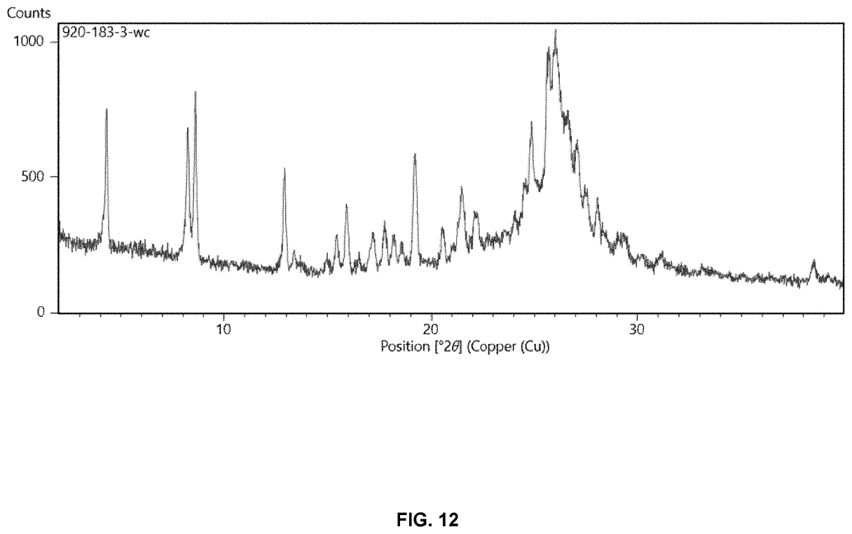

In embodiments, Form L is characterized by an XRPD pattern substantially similar to that shown in FIG. 12.

In embodiments, Form L is characterized by an XRPD pattern comprising peaks shown in Table 15.

In embodiments, Form L is characterized one, two, three, four, five, six, seven, eight, nine, ten, eleven, or more, XRPD peaks listed in Table 15.

### XII. Form M

In embodiments, the crystalline form of Compound I is Form M.

In embodiments, Form M is characterized by peaks in an XRPD pattern at 7.3±0.2, 10.4±0.2, and 12.7±0.2 °2θ. In embodiments, the variance at any of these peaks is +0.1 °2θ.

In embodiments, Form M is characterized by peaks in an XRPD pattern at 7.3±0.2, 9.2±0.2, 10.4±0.2, 11.8±0.2, 12.7±0.2, 14.8±0.2, 16.2±0.2, 17.5±0.2, 18.6±0.2, 19.0±0.2, 19.9±0.2, 21.0±0.2, 22.3±0.2, 23.6±0.2, 29.2±0.2, 31.2±0.2, 32.5±0.2, and 33.5±0.2 °2θ. In embodiments, the variance at any of these peaks is +0.1 °2θ.

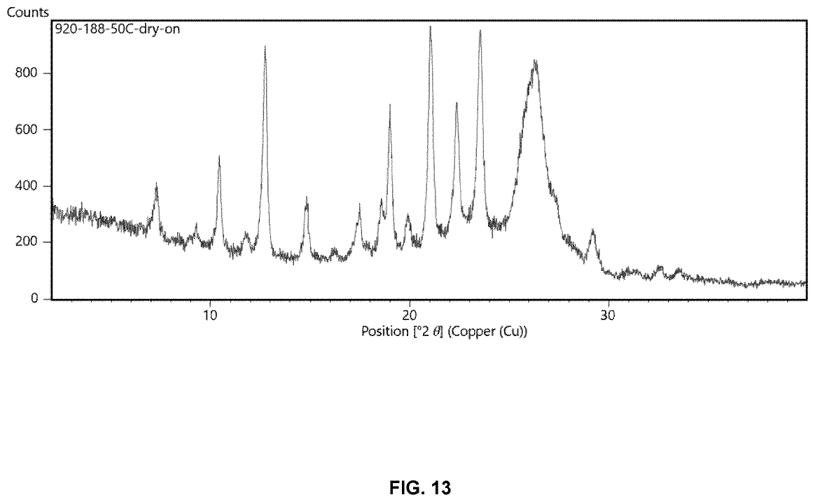

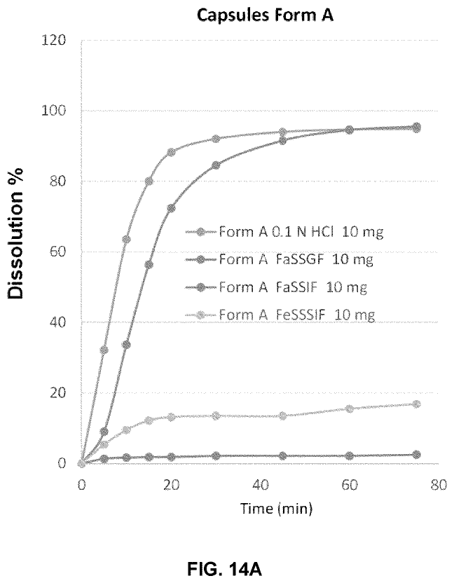

In embodiments, Form M is characterized by an XRPD pattern substantially similar to that shown in FIG. 13.

In embodiments, Form M is characterized by an XRPD pattern comprising peaks shown in Table 16.

In embodiments, Form M is characterized one, two, three, four, five, six, seven, eight, nine, ten, eleven, or more, XRPD peaks listed in Table 16.

### Pharmaceutical Compositions

Aspects of the present disclosure relate to pharmaceutical compositions comprising a therapeutically effective amount of at least one crystalline form of Compound I (e.g., Form A, Form B, Form C, Form D, Form E, Form F, Form G, Form H, Form I, Form J, Form K, Form L, or Form M) or salt, solvate, or solvate salt thereof, and a pharmaceutically acceptable carrier or excipient.

The dosage of the crystalline form of Compound I (e.g., Form A, Form B, Form C, Form D, Form E, Form F, Form G, Form H, Form I, Form J, Form K, Form L, or Form M) or salt, solvate, or solvate salt thereof will, of course, vary with the mode of administration, the treatment desired and the disorder indicated. For example, if Compound I administered orally, then the daily dosage of the compound of may be in the range from 0.01 micrograms per kilogram body weight (g/kg) to 100 milligrams per kilogram body weight (mg/kg).

A crystalline form of Compound I (e.g., Form A, Form B, Form C, Form D, Form E, Form F, Form G, Form H, Form I, Form J, Form K, Form L, or Form M) or salt, solvate, or solvate salt thereof, may be used on their own but will generally be administered in the form of a pharmaceutical composition in which the compounds, are in association with a pharmaceutically acceptable adjuvant, diluent or carrier. Conventional procedures for the selection and preparation of suitable pharmaceutical formulations are described in, for example, “Pharmaceuticals—The Science of Dosage Form Designs”, M. E. Aulton, Churchill Livingstone, 1988.

Depending on the mode of administration, the pharmaceutical composition which is used to administer will comprise from 0.05 to 99% w (percent by weight), 0.05 to 80%, 0.10 to 70%, or 0.10 to 50% of the crystalline forms of Compound I (e.g., Form A, Form B, Form C, Form D, Form E, Form F, Form G, Form H, Form I, Form J, Form K, Form L, or Form M) or salt, solvate, or solvate salt thereof, all percentages by weight being based on total composition.

The pharmaceutical compositions may be administered topically (e.g., to the skin) in the form, e.g., of creams, gels, lotions, solutions, suspensions, or systemically, e.g., by oral administration in the form of tablets, capsules, syrups, powders or granules; or by parenteral administration in the form of a sterile solution, suspension or emulsion for injection (including intravenous, subcutaneous, intramuscular, intravascular or infusion); by rectal administration in the form of suppositories; or by inhalation in the form of an aerosol.

For oral administration the crystalline forms of Compound I (e.g., Form A, Form B, Form C, Form D, Form E, Form F, Form G, Form H, Form I, Form J, Form K, Form L, or Form M) or salt, solvate, or solvate salt thereof may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the crystalline forms of Compound I (e.g., Form A, Form B, Form C, Form D, Form E, Form F, Form G, Form H, Form I, Form J, Form K, Form L, or Form M) or salt, solvate, or solvate salt thereof may be admixed with, for example, a vegetable oil or polyethylene glycol. Hard gelatine capsules may contain granules of the compound using either of the above-mentioned excipients for tablets. Also liquid or semisolid formulations of the crystalline forms of Compound I (e.g., Form A, Form B, Form C, Form D, Form E, Form F, Form G, Form H, Form I, Form J, Form K, Form L, or Form M) or salt, solvate, or solvate salt thereof may be filled into hard gelatin capsules. Liquid preparations for oral application may be in the form of syrups or suspensions, for example, solutions containing the crystalline forms of Compound I (e.g., Form A, Form B, Form C, Form D, Form E, Form F, Form G, Form H, Form I, Form J, Form K, Form L, or Form M) or salt, solvate, or solvate salt thereof, the balance being sugar and a mixture of ethanol, water, glycerol and propylene glycol. Optionally such liquid preparations may contain coloring agents, flavoring agents, sweetening agents (such as saccharine), preservative agents and/or carboxymethylcellulose as a thickening agent or other excipients known to those skilled in art.

For intravenous (parenteral) administration the crystalline forms of Compound I (e.g., Form A, Form B, Form C, Form D, Form E, Form F, Form G, Form H, Form I, Form J, Form K, Form L, or Form M) or salt, solvate, or solvate salt thereof may be administered as a sterile aqueous or oily solution.

The size of the dose for therapeutic purposes of the crystalline forms of Compound I (e.g., Form A, Form B, Form C, Form D, Form E, Form F, Form G, Form H, Form I, Form J, Form K, Form L, or Form M) or salt, solvate, or solvate salt thereof will naturally vary according to the nature and severity of the conditions, the age and sex of the animal or patient and the route of administration, according to well-known principles of medicine.

Dosage levels, dose frequency, and treatment durations of the crystalline forms of Compound I (e.g., Form A, Form B, Form C, Form D, Form E, Form F, Form G, Form H, Form I, Form J, Form K, Form L, or Form M) or salt, solvate, or solvate salt thereof are expected to differ depending on the formulation and clinical indication, age, and co-morbid medical conditions of the patient. The standard duration of treatment with the crystalline forms of Compound I (e.g., Form A, Form B, Form C, Form D, Form E, Form F, Form G, Form H, Form I, Form J, Form K, Form L, or Form M) or salt, solvate, or solvate salt thereof is expected to vary between one and seven days for most clinical indications. It may be necessary to extend the duration of treatment beyond seven days in instances of recurrent infections or infections associated with tissues or implanted materials to which there is poor blood supply including bones/joints, respiratory tract, endocardium, and dental tissues.

### Therapeutic Methods

Aspects of the present disclosure relate to methods of using at least one crystalline form of Compound I (e.g., Form A, Form B, Form C, Form D, Form E, Form F, Form G, Form H, Form I, Form J, Form K, Form L, or Form M) or a salt, solvate, or solvate salt thereof for treating various diseases and conditions. Aspects of the present disclosure also relate to methods of treating various diseases or conditions, comprising administering at least one crystalline form of Compound I (e.g., Form A, Form B, Form C, Form D, Form E, Form F, Form G, Form H, Form I, Form J, Form K, Form L, or Form M) or a salt, solvate, or solvate salt thereof to a subject in need thereof.

In embodiments, the methods or uses disclosed herein comprise administering crystalline forms of Compound I (e.g., Form A, Form B, Form C, Form D, Form E, Form F, Form G, Form H, Form I, Form J, Form K, Form L, or Form M) or a salt, solvate, or solvate salt thereof, for treating a disease or a condition implicated by abnormal activity of one or more RAF kinase. In embodiments, the methods or uses comprise administering crystalline forms of Compound I (e.g., Form A, Form B, Form C, Form D, Form E, Form F, Form G, Form H, Form I, Form J, Form K, Form L, or Form M) or a salt, solvate, or solvate salt thereof, for treating a disease or a condition treatable by the inhibition of one or more Raf kinase. RAF kinase inhibition is relevant for the treatment of many different diseases associated with the abnormal activity of the MAPK pathway. In embodiments the condition treatable by the inhibition of RAF kinases, such as B-RAF or C-RAF.

In embodiments, the disease or the condition is cancer. In embodiments, the disease or the condition is selected from Barret's adenocarcinoma; biliary tract carcinomas; breast cancer; cervical cancer; cholangiocarcinoma; central nervous system tumors; primary CNS tumors; glioblastomas, astrocytomas; glioblastoma multiforme; ependymomas; secondary CNS tumors (metastases to the central nervous system of tumors originating outside of the central nervous system); brain tumors; brain metastases; colorectal cancer; large intestinal colon carcinoma; gastric cancer; carcinoma of the head and neck; squamous cell carcinoma of the head and neck; acute lymphoblastic leukemia; acute myelogenous leukemia (AML); myelodysplastic syndromes; chronic myelogenous leukemia; Hodgkin's lymphoma; non-Hodgkin's lymphoma; megakaryoblastic leukemia; multiple myeloma; erythroleukemia; hepatocellular carcinoma; lung cancer; small cell lung cancer; non-small cell lung cancer; ovarian cancer; endometrial cancer; pancreatic cancer; pituitary adenoma; prostate cancer; renal cancer; metastatic melanoma or thyroid cancers.

In embodiments, the disease or the condition is melanoma, non-small cell cancer, colorectal cancer, ovarian cancer, thyroid cancer, breast cancer or cholangiocarcinoma. In embodiments, the disease or the condition is colorectal cancer. In embodiments, the disease or the condition is melanoma.

In embodiments, the present disclosure provides methods of treating small lung cancer, comprising administering at least one crystalline form of Compound I (e.g., Form A, Form B, Form C, Form D, Form E, Form F, Form G, Form H, Form I, Form J, Form K, Form L, or Form M) or a salt, solvate, or solvate salt thereof to a subject in need thereof.

In embodiments, the present disclosure provides methods of treating colorectal cancer, comprising administering at least one crystalline form of Compound I (e.g., Form A, Form B, Form C, Form D, Form E, Form F, Form G, Form H, Form I, Form J, Form K, Form L, or Form M) or a salt, solvate, or solvate salt thereof to a subject in need thereof.

In embodiments, the present disclosure provides methods of treating melanoma, comprising administering at least one crystalline form of Compound I (e.g., Form A, Form B, Form C, Form D, Form E, Form F, Form G, Form H, Form I, Form J, Form K, Form L, or Form M) or a salt, solvate, or solvate salt thereof to a subject in need thereof.

In embodiments, the present disclosure provides methods of treating pancreatic cancer, comprising administering at least one crystalline form of Compound I (e.g., Form A, Form B, Form C, Form D, Form E, Form F, Form G, Form H, Form I, Form J, Form K, Form L, or Form M) or a salt, solvate, or solvate salt thereof to a subject in need thereof.

In embodiments, the disease or the condition is cancer comprising a BRAF^{V600E} mutation. In embodiments, the disease or the condition is modulated by BRAFV^{600E} In embodiments, the disease or the condition is BRAF^{V600E} melanoma, BRAF^{V600E} colorectal cancer, BRAF^{V600E} papillary thyroid cancers, BRAFV^{600E} low grade serous ovarian cancers, BRAFV^{600E} glioma, BRAF^{V600E} hepatobiliary cancers, BRAF^{V600E} hairy cell leukaemia, BRAF^{V600E} non-small cell cancer, or BRAF^{V600E} pilocytic astrocytoma.

In embodiments, the disease or the condition is cardio-facio cutaneous syndrome and polycystic kidney disease.

In embodiments, crystalline forms of Compound I (e.g., Form A, Form B, Form C, Form D, Form E, Form F, Form G, Form H, Form I, Form J, Form K, Form L, or Form M) or a salt, solvate, or solvate salt thereof are used in combination with one or more pharmaceutically active agents.

In embodiments, pharmaceutically active agent is an anti-tumor agent.

In embodiments, the additional pharmaceutically active agent is an antiproliferative/antineoplastic drug. In embodiments, antiproliferative/antineoplastic drug is alkylating agent (for example cis-platin, oxaliplatin, carboplatin, cyclophosphamide, nitrogen mustard, bendamustin, melphalan, chlorambucil, busulphan, temozolamide and nitrosoureas); antimetabolite (for example gemcitabine and antifolates such as fluoropyrimidines like 5-fluorouracil and tegafur, raltitrexed, methotrexate, pemetrexed, cytosine arabinoside, and hydroxyurea); antibiotic (for example anthracyclines like adriamycin, bleomycin, doxorubicin, daunomycin, epirubicin, idarubicin, mitomycin-C, dactinomycin and mithramycin); antimitotic agent (for example vinca alkaloids like vincristine, vinblastine, vindesine and vinorelbine and taxoids like taxol and taxotere and polokinase inhibitors); proteasome inhibitor, for example carfilzomib and bortezomib; interferon therapy; or topoisomerase inhibitor (for example epipodophyllotoxins like etoposide and teniposide, amsacrine, topotecan, mitoxantrone and camptothecin).

In embodiments, the additional pharmaceutically active agent is a cytostatic agent. In embodiments, cytostatic agent is antiestrogen (for example tamoxifen, fulvestrant, toremifene, raloxifene, droloxifene and iodoxyfene), antiandrogen (for example bicalutamide, flutamide, nilutamide and cyproterone acetate), LHRH antagonist or LHRH agonist (for example goserelin, leuprorelin and buserelin), progestogen (for example megestrol acetate), aromatase inhibitor (for example as anastrozole, letrozole, vorazole and exemestane) or inhibitor of 5α-reductase such as finasteride.

In embodiments, the additional pharmaceutically active agent is an anti-invasion agent. In embodiments, the anti-invasion agent is dasatinib and bosutinib (SKI-606), metalloproteinase inhibitor, or inhibitor of urokinase plasminogen activator receptor function or antibody to Heparanase.

In embodiments, the additional pharmaceutically active agent is an inhibitor of growth factor function. In embodiments, the inhibitor of growth factor function is growth factor antibody and growth factor receptor antibody, for example the anti-erbB2 antibody trastuzumab [Herceptin™], the anti-EGFR antibody panitumumab, the anti-erbB1 antibody cetuximab, tyrosine kinase inhibitor, for example inhibitors of the epidermal growth factor family (for example EGFR family tyrosine kinase inhibitor such as gefitinib, erlotinib and 6-acrylamido-N-(3-chloro-4-fluorophenyl)-7-(3-morpholinopropoxy)-quinazolin-4-amine (CI 1033), erbB2 tyrosine kinase inhibitor such as lapatinib); inhibitor of the hepatocyte growth factor family; inhibitor of the insulin growth factor family; modulator of protein regulators of cell apoptosis (for example Bcl-2 inhibitors); inhibitor of the platelet-derived growth factor family such as imatinib and/or nilotinib (AMN107); inhibitor of serine/threonine kinases (for example Ras/RAF signaling inhibitors such as farnesyl transferase inhibitor, for example sorafenib, tipifarnib and lonafarnib), inhibitor of cell signaling through MEK and/or AKT kinase, c-kit inhibitor, abl kinase inhibitor, PI3 kinase inhibitor, Plt3 kinase inhibitor, CSF-1R kinase inhibitor, IGF receptor, kinase inhibitor; aurora kinase inhibitor or cyclin dependent kinase inhibitor such as CDK2 and/or CDK4 inhibitor.

In embodiments, the additional pharmaceutically active agent is an antiangiogenic agent. In embodiments, the antiangiogenic agent inhibits the effects of vascular endothelial growth factor, for example the anti-vascular endothelial cell growth factor antibody bevacizumab (Avastin™); thalidomide; lenalidomide; and for example, a VEGF receptor tyrosine kinase inhibitor such as vandetanib, vatalanib, sunitinib, axitinib and pazopanib.

In embodiments, the additional pharmaceutically active agent is a cIn embodiments, the cytotoxic agent is fludaribine (fludara), cladribine, or pentostatin (Nipent™)

In embodiments, the additional pharmaceutically active agent is a steroid. In embodiments, the steroid is corticosteroid, including glucocorticoid and mineralocorticoid, for example aclometasone, aclometasone dipropionate, aldosterone, amcinonide, beclomethasone, beclomethasone dipropionate, betamethasone, betamethasone dipropionate, betamethasone sodium phosphate, betamethasone valerate, budesonide, clobetasone, clobetasone butyrate, clobetasol propionate, cloprednol, cortisone, cortisone acetate, cortivazol, deoxycortone, desonide, desoximetasone, dexamethasone, dexamethasone sodium phosphate, dexamethasone isonicotinate, difluorocortolone, fluclorolone, flumethasone, flunisolide, fluocinolone, fluocinolone acetonide, fluocinonide, fluocortin butyl, fluorocortisone, fluorocortolone, fluocortolone caproate, fluocortolone pivalate, fluorometholone, fluprednidene, fluprednidene acetate, flurandrenolone, fluticasone, fluticasone propionate, halcinonide, hydrocortisone, hydrocortisone acetate, hydrocortisone butyrate, hydrocortisone aceponate, hydrocortisone buteprate, hydrocortisone valerate, icomethasone, icomethasone enbutate, meprednisone, methylprednisolone, mometasone paramethasone, mometasone furoate monohydrate, prednicarbate, prednisolone, prednisone, tixocortol, tixocortol pivalate, triamcinolone, triamcinolone acetonide, triamcinolone alcohol and their respective pharmaceutically acceptable derivatives. A combination of steroids may be used, for example a combination of two or more steroids as described herein.

In embodiments, the additional pharmaceutically active agent is a targeted therapy agent. In embodiments, the targeted therapy agent is a PI3Kd inhibitor, for example idelalisib and perifosine.

In embodiments, the additional pharmaceutically active agent is an immunotherapeutic agent. In embodiments, the immunotherapeutic agent is antibody therapy agent such as alemtuzumab, rituximab, ibritumomab tiuxetan (Zevalin®) and ofatumumab; interferon such as interferon a; interleukins such as IL-2 (aldesleukin); interleukin inhibitors for example IRAK4 inhibitors; cancer vaccine including prophylactic and treatment vaccines such as HPV vaccines, for example Gardasil, Cervarix, Oncophage and Sipuleucel-T (Provenge); toll-like receptor modulator for example TLR-7 or TLR-9 agonist; and PD-1 antagonist, PDL-1 antagonist, and IDO-1 antagonist.

In embodiments, the pharmaceutical composition may be used in combination with another therapy. In embodiments, the other therapy is gene therapy, including for example approaches to replace aberrant genes such as aberrant p53 or aberrant BRCA1 or BRCA2.

In embodiments, the other therapy is immunotherapy approaches, including for example antibody therapy such as alemtuzumab, rituximab, ibritumomab tiuxetan (Zevalin®) and ofatumumab; interferons such as interferon a; interleukins such as IL-2 (aldesleukin); interleukin inhibitors for example IRAK4 inhibitors; cancer vaccines including prophylactic and treatment vaccines such as HPV vaccines, for example Gardasil, Cervarix, Oncophage and Sipuleucel-T (Provenge); toll-like receptor modulators for example TLR-7 or TLR-9 agonists; and PD-1 antagonists, PDL-1 antagonists, and IDO-1 antagonists.

### EXAMPLES

Compound I can be prepared according to the methods disclosed in WIPO Patent Appln Pub. No. 2022/023450, which is incorporated herein for all purposes. The following examples are used to assist in describing the present disclosure, but should not be seen as limiting the present disclosure in any way.

### Example 1

Unless otherwise specified, all characterization data were obtained using the following procedures:

### X-Ray Powder Diffraction (XRPD)

XRPD analysis was carried out on a Bruker D8 Advance with LYNXYE_XE_T ID detector, scanning the samples between 3 and 400 20. The material was ground on a monocrystalline silicon holder for analysis. The material was then analyzed using Cu K radiation (α1λ=1.54060 A; α2=1.54443 A; β=1.39225 Å; α1:α2 ratio=0.5) and step size of 0.02° and 0.12s/step scanning time was applied. Primary beam path slit was 10.0 mm by sample length and SollerMount axial soller was 2.5°. Secondary beam path slit was 5.2 mm.

### Differential Scanning Calorimetry (DSC)

Approximately 1-2 mg of material was weighed into an aluminum DSC pan with a pin hold. The sample pan then placed on Differential Scanning Calorimetry (TA Discovery 2500) with protected nitrogen flow rate of 50 mL/min. The sample was heated at rate of 10° C./min from 30° C. to 250° C. during which heat flow was recorded.

### Thermogravimetric Analysis (TGA)

Approximately 2 mg of material was weighed into an open aluminum pan and loaded into a simultaneous thermogravimetric/differential thermal analyzer (TG Discovery 5500) and held at room temperature. Nitrogen flow rate for balance was 10 mL/min and nitrogen flow rate for sample was 25 mL/min. The sample was then heated at a rate of 10° C./min from 30° C. to 300° C. during which time the change in sample weight was recorded.

### Dynamic Vapor Sorption (DVS)

Approximately 10 mg of material was analyzed using an automated dynamic vapor sorption analyzer (Intrinsic/Advantage) at 25° C. Method 1 for anhydrate undergo humidity cycle of 40-0-95-0-40% RH with stage step of 10% RH during which mass change was recorded. Method 2 for hydrate undergo humidity cycle of 40-95-0-95-40% RH with stage step of 10% RH during which mass change we recorded. Equilibrium set up was 0.002 dm/dt (%/min). Minimum dm/dt stability during in 60 min and maximum dm/dt stability duration is 360 min.

### Form A

Compound 1 was dissolved in DMSO (18 vol) at 25° C. Water (2.5 vol) is added, followed by 0.5% Compound 1 Form A seed. The slurry was stirred for 2 hours at 25° C. Water (6.5 vol) is added over 6 hours, and the slurry stirred for 12 hours at 25° C. The slurry was filtered and the solid washed with water (5.6 vol) four times. The solid material was then slurried in water (15 vol) for 18 hours at 25° C. The solid was washed with water (5.6 vol) three times before drying under vacuum at 40° C. Form A was then characterized by XRPD (FIG. 1A, Table 1), DSC (FIG. 1B), TGA (FIG. 1C), and DVS (FIG. 1D, Table 2).

A summary of the XRPD peaks of Form A is found below in Table 1.

TABLE 1
XRPD Peaks of Form A

| No. | 2-Theta | d(Å) | Relative Intensity (%) | Intensity |
|---|---|---|---|---|
| 1 | 5.75 | 15.36 | 46.93 | 576.81 |
| 2 | 5.98 | 14.77 | 16.48 | 202.58 |
| 3 | 7.08 | 12.47 | 15.93 | 195.79 |
| 4 | 9.30 | 9.50 | 4.96 | 60.91 |
| 5 | 11.40 | 7.76 | 26.80 | 329.39 |
| 6 | 11.76 | 7.52 | 63.89 | 785.25 |
| 7 | 14.13 | 6.26 | 7.04 | 86.55 |
| 8 | 16.58 | 5.34 | 29.11 | 357.79 |
| 9 | 17.26 | 5.13 | 55.82 | 686.12 |
| 10 | 17.49 | 5.07 | 28.01 | 344.23 |
| 11 | 17.89 | 4.95 | 27.62 | 339.48 |
| 12 | 18.47 | 4.80 | 20.48 | 251.76 |
| 13 | 19.14 | 4.63 | 8.35 | 102.58 |
| 14 | 19.80 | 4.48 | 14.30 | 175.78 |
| 15 | 20.71 | 4.29 | 4.64 | 57.06 |
| 16 | 21.28 | 4.17 | 7.09 | 87.20 |
| 17 | 21.56 | 4.12 | 13.64 | 167.61 |
| 18 | 22.13 | 4.01 | 20.44 | 251.21 |
| 19 | 23.14 | 3.84 | 100.00 | 1229.08 |
| 20 | 24.18 | 3.68 | 93.02 | 1143.27 |
| 21 | 25.59 | 3.48 | 11.43 | 140.45 |
| 22 | 26.10 | 3.41 | 12.46 | 153.16 |
| 23 | 26.51 | 3.36 | 5.73 | 70.37 |
| 24 | 27.26 | 3.27 | 4.03 | 49.53 |
| 25 | 28.35 | 3.15 | 13.66 | 167.87 |
| 26 | 35.81 | 2.51 | 3.73 | 45.85 |

A summary of the DVS isotherm analysis of Form A is found below in Table 2.

TABLE 2
DVS Isotherm Analysis of Form A

| | Target | Change In Mass (%) - ref | | |
|---|---|---|---|---|
| | % P/Po | Sorption | Desorption | Hysteresis |
| Cycle 1 | 0.0 | — | 0.002 | — |
| | 10.0 | — | 0.350 | — |
| | 20.0 | — | 0.941 | — |
| | 30.0 | — | 3.649 | — |
| | 40.0 | 4.623 | 4.700 | 0.077 |
| | 50.0 | 5.112 | 5.395 | 0.283 |
| | 60.0 | 5.475 | 5.814 | 0.339 |
| | 70.0 | 5.845 | 6.213 | 0.368 |
| | 80.0 | 6.194 | 6.535 | 0.341 |
| | 90.0 | 6.594 | 6.824 | 0.230 |
| | 95.0 | 6.838 | 6.838 | — |
| Cycle 2 | 0.0 | 0.002 | — | — |
| | 10.0 | 0.312 | — | — |
| | 20.0 | 0.853 | — | — |
| | 30.0 | 3.253 | — | — |
| | 40.0 | 4.225 | 4.601 | 0.375 |
| | 50.0 | 4.800 | 5.295 | 0.495 |
| | 60.0 | 5.248 | 5.715 | 0.466 |
| | 70.0 | 5.706 | 6.129 | 0.423 |
| | 80.0 | 6.122 | 6.446 | 0.324 |
| | 90.0 | 6.536 | 6.737 | 0.201 |
| | 95.0 | 6.745 | 6.745 | — |

Temperature 24.8° C.
MRef: 11.5362 from Custom Mass

### Form A *

Compound 1 Form A was equilibrated in acetone at 25° C. for 2 weeks with stirring. The resultant suspension was filtered. The wet cake was analyzed and Form A* was then characterized by XRPD (FIG. 1E, Table 3) and DSC (FIG. 1F).

A summary of the XRPD peaks of Form A* is found below in Table 3.

TABLE 3
XRPD Peaks of Form A*

| No. | 2-Theta | d(Å) | Relative Intensity (%) | Intensity |
|---|---|---|---|---|
| 1 | 5.71 | 15.47 | 55.36 | 349.83 |
| 2 | 5.91 | 14.95 | 22.03 | 139.23 |
| 3 | 7.09 | 12.46 | 16.06 | 101.50 |
| 4 | 9.25 | 9.55 | 8.13 | 51.39 |
| 5 | 11.41 | 7.75 | 29.58 | 186.91 |
| 6 | 11.75 | 7.52 | 69.27 | 437.74 |
| 7 | 14.09 | 6.28 | 4.79 | 30.28 |
| 8 | 16.63 | 5.33 | 28.22 | 178.33 |
| 9 | 17.22 | 5.15 | 59.66 | 376.99 |
| 10 | 17.48 | 5.07 | 28.32 | 178.96 |
| 11 | 17.78 | 4.98 | 29.63 | 187.20 |
| 12 | 18.53 | 4.79 | 17.58 | 111.10 |
| 13 | 19.14 | 4.63 | 10.64 | 67.20 |
| 14 | 19.73 | 4.50 | 14.74 | 93.12 |
| 15 | 20.58 | 4.31 | 5.74 | 36.30 |
| 16 | 21.42 | 4.14 | 19.92 | 125.90 |
| 17 | 22.07 | 4.02 | 22.78 | 143.95 |
| 18 | 22.09 | 4.02 | 20.90 | 132.04 |
| 19 | 23.06 | 3.85 | 100.00 | 631.89 |
| 20 | 23.50 | 3.78 | 13.52 | 85.44 |
| 21 | 24.11 | 3.69 | 90.65 | 572.79 |
| 22 | 25.48 | 3.49 | 7.32 | 46.27 |
| 23 | 27.24 | 3.27 | 3.77 | 23.81 |
| 24 | 28.27 | 3.15 | 16.50 | 104.26 |
| 25 | 28.95 | 3.08 | 4.06 | 25.63 |
| 26 | 30.80 | 2.90 | 4.22 | 26.66 |

### Form B

Compound 1 Phosphate salt Form A was equilibrated in water (2 mg/mL concentration) at 25° C. for 2 days. The resultant suspension was filtered, and the wet cake was analyzed. Form B was then characterized by XRPD (FIG. 2A, Table 4), DSC (FIG. 2B), and TGA (FIG. 2C).

A summary of the XRPD peaks of Form B is found below in Table 4.

TABLE 4
XRPD Peaks of Form B

| No. | 2-Theta | d(Å) | Relative Intensity (%) | Intensity |
|---|---|---|---|---|
| 1 | 6.19 | 14.26 | 36.34 | 38.80 |
| 2 | 6.24 | 14.15 | 46.97 | 50.15 |
| 3 | 12.66 | 6.99 | 100.00 | 106.77 |
| 4 | 13.40 | 6.60 | 51.96 | 55.48 |
| 5 | 13.62 | 6.49 | 33.99 | 36.30 |
| 6 | 18.96 | 4.68 | 24.74 | 26.42 |
| 7 | 19.82 | 4.48 | 20.41 | 21.79 |
| 8 | 23.33 | 3.81 | 41.54 | 44.35 |
| 9 | 23.44 | 3.79 | 53.96 | 57.62 |
| 10 | 26.07 | 3.42 | 27.22 | 29.07 |
| 11 | 26.32 | 3.38 | 28.03 | 29.93 |
| 12 | 39.11 | 2.30 | 20.84 | 22.26 |

### Form C

Compound 1 Form A was equilibrated in DMSO with stirring at 25° C. over 2 weeks. The precipitate was filtered, and resultant Form C was then characterized by XRPD (FIG. 3A, Table 5), DSC (FIG. 3B), and TGA (FIG. 3C).

A summary of the XRPD peaks of Form C is found below in Table 5.

TABLE 5
XRPD Peaks of Form C

| No. | 2-Theta | d(Å) | Relative Intensity (%) | Intensity |
|---|---|---|---|---|
| 1 | 3.63 | 24.33 | 4.27 | 34.80 |
| 2 | 4.22 | 20.93 | 2.13 | 17.40 |
| 3 | 5.81 | 15.19 | 28.40 | 231.65 |
| 4 | 7.07 | 12.49 | 10.64 | 86.79 |
| 5 | 9.43 | 9.37 | 4.39 | 35.77 |
| 6 | 11.50 | 7.69 | 38.73 | 315.93 |
| 7 | 11.70 | 7.56 | 42.57 | 347.25 |
| 8 | 12.77 | 6.92 | 4.33 | 35.34 |
| 9 | 14.14 | 6.26 | 10.69 | 87.23 |
| 10 | 16.81 | 5.27 | 77.49 | 632.10 |
| 11 | 17.20 | 5.15 | 54.85 | 447.38 |
| 12 | 17.47 | 5.07 | 31.96 | 260.70 |
| 13 | 17.88 | 4.96 | 25.75 | 210.03 |
| 14 | 18.21 | 4.87 | 19.00 | 154.96 |
| 15 | 18.43 | 4.81 | 38.08 | 310.61 |
| 16 | 18.95 | 4.68 | 12.34 | 100.64 |
| 17 | 19.63 | 4.52 | 20.78 | 169.53 |
| 18 | 20.44 | 4.34 | 7.21 | 58.78 |
| 19 | 21.28 | 4.17 | 15.39 | 125.56 |
| 20 | 22.06 | 4.03 | 21.72 | 177.19 |
| 21 | 22.55 | 3.94 | 15.07 | 122.90 |
| 22 | 23.10 | 3.85 | 100.00 | 815.70 |
| 23 | 23.37 | 3.80 | 25.83 | 210.69 |
| 24 | 24.19 | 3.68 | 95.32 | 777.49 |
| 25 | 25.65 | 3.47 | 11.65 | 95.05 |
| 26 | 26.17 | 3.40 | 17.74 | 144.67 |
| 27 | 26.58 | 3.35 | 5.14 | 41.95 |
| 28 | 28.53 | 3.13 | 20.09 | 163.89 |
| 29 | 30.25 | 2.95 | 5.00 | 40.81 |
| 30 | 30.96 | 2.89 | 6.11 | 49.85 |
| 31 | 32.38 | 2.76 | 6.40 | 52.22 |
| 32 | 33.54 | 2.67 | 4.82 | 39.34 |
| 33 | 36.16 | 2.48 | 8.64 | 70.45 |

### Form D

Form A was dissolved in the minimum amount of DMF to form a saturated solution. Water was added slowly to form a suspension (1:3 DMF:water). The precipitate was filtered, and the wet cake analyzed. Form D was then characterized by XRPD (FIG. 4A, Table 6), DSC (FIG. 4B), and TGA (FIG. 4C).

A summary of the XRPD peaks of Form D is found below in Table 6.

TABLE 6
XRPD Peaks of Form D

| No. | 2-Theta | d(Å) | Relative Intensity (%) | Intensity |
|---|---|---|---|---|
| 1 | 5.79 | 15.27 | 90.69 | 180.70 |
| 2 | 6.04 | 14.62 | 31.57 | 62.91 |
| 3 | 7.16 | 12.34 | 12.95 | 25.80 |
| 4 | 8.85 | 9.99 | 9.86 | 19.64 |
| 5 | 11.65 | 7.59 | 47.62 | 94.88 |
| 6 | 11.80 | 7.49 | 100.00 | 199.25 |
| 7 | 12.47 | 7.09 | 56.01 | 111.59 |
| 8 | 13.04 | 6.79 | 23.42 | 46.67 |
| 9 | 16.92 | 5.24 | 30.53 | 60.83 |
| 10 | 17.28 | 5.13 | 53.57 | 106.75 |
| 11 | 17.75 | 4.99 | 30.96 | 61.69 |
| 12 | 18.53 | 4.79 | 30.20 | 60.16 |
| 13 | 19.10 | 4.64 | 14.73 | 29.36 |
| 14 | 19.73 | 4.50 | 16.46 | 32.80 |
| 15 | 21.29 | 4.17 | 11.75 | 23.40 |
| 16 | 23.12 | 3.84 | 95.98 | 191.25 |
| 17 | 23.41 | 3.80 | 87.00 | 173.35 |
| 18 | 24.10 | 3.69 | 70.55 | 140.58 |
| 19 | 26.19 | 3.40 | 14.85 | 29.59 |
| 20 | 26.37 | 3.38 | 27.75 | 55.29 |

### Form E

Compound 1 Form A was dissolved in the minimum amount of acetone. The solution was filtered through a 0.45 m syringe membrane filter. The clear solution was then slowly evaporated at ambient condition. The solid residue was analyzed, and Form E was then characterized by XRPD (FIG. 5A, Table 7), DSC (FIG. 5B), and TGA (FIG. 5C).

A summary of the XRPD peaks of Form E is found below in Table 7.

TABLE 7
XRPD Peaks of Form E

| No. | 2-Theta | d(Å) | Relative Intensity (%) | Intensity |
|---|---|---|---|---|
| 1 | 5.80 | 15.22 | 100.00 | 1202.00 |
| 2 | 7.04 | 12.55 | 8.07 | 96.97 |
| 3 | 9.39 | 9.41 | 4.00 | 48.05 |
| 4 | 11.44 | 7.73 | 29.25 | 351.59 |
| 5 | 11.63 | 7.60 | 37.05 | 445.37 |
| 6 | 14.06 | 6.30 | 20.89 | 251.06 |
| 7 | 16.73 | 5.29 | 71.48 | 859.18 |
| 8 | 17.10 | 5.18 | 65.25 | 784.29 |
| 9 | 17.41 | 5.09 | 84.60 | 1016.95 |
| 10 | 17.80 | 4.98 | 21.95 | 263.86 |
| 11 | 18.11 | 4.90 | 15.72 | 188.99 |
| 12 | 18.32 | 4.84 | 36.09 | 433.85 |
| 13 | 18.76 | 4.73 | 13.99 | 168.20 |
| 14 | 19.38 | 4.58 | 19.50 | 234.44 |
| 15 | 20.17 | 4.40 | 8.31 | 99.94 |
| 16 | 20.88 | 4.25 | 3.18 | 38.23 |
| 17 | 21.13 | 4.20 | 15.92 | 191.39 |
| 18 | 21.81 | 4.07 | 15.05 | 180.94 |
| 19 | 22.10 | 4.02 | 11.43 | 137.42 |
| 20 | 22.85 | 3.89 | 95.63 | 1149.46 |
| 21 | 23.26 | 3.82 | 59.68 | 717.41 |
| 22 | 23.95 | 3.71 | 70.19 | 843.69 |
| 23 | 25.36 | 3.51 | 5.67 | 68.15 |
| 24 | 25.90 | 3.44 | 15.59 | 187.44 |
| 25 | 26.34 | 3.38 | 4.41 | 53.04 |
| 26 | 28.30 | 3.15 | 21.06 | 253.19 |
| 27 | 33.27 | 2.69 | 3.62 | 43.52 |
| 28 | 35.73 | 2.51 | 5.86 | 70.49 |

### Form F

Compound 1 Form A was dissolved in 11.5 vol of DMSO/ethyl acetate (1:1) at 70° C. The solution was polish filtered and washed with 0.5 vol DMSO/ethyl acetate (1:1). The solution was cooled to 60° C. over 30 minutes and methanol (4 vol) was added. 0.5% o Form F seed was added, and the slurry was stirred for 4 hours at 60° C. Methanol (12 vol) was added over 6 hours. The slurry was then cooled to 10° C. over 5 hours, and held for 9 hours at 10° C. The slurry was filtered and washed twice with methanol (4 volumes). Form F was then characterized by XRPD (FIG. 6A, Table 8), DSC (FIG. 6B), TGA (FIG. 6C), and DVS (FIG. 6D, Table 9).

A summary of the XRPD peaks of Form F is found below in Table 8.

TABLE 8
XRPD Peaks of Form F

| No. | 2-Theta | d(Å) | Relative Intensity (%) | Intensity |
|---|---|---|---|---|
| 1 | 11.78 | 7.50 | 3.85 | 92.42 |
| 2 | 12.96 | 6.83 | 62.93 | 1510.88 |
| 3 | 13.88 | 6.38 | 3.82 | 91.75 |
| 4 | 14.17 | 6.24 | 20.59 | 494.25 |
| 5 | 14.50 | 6.11 | 6.70 | 160.81 |
| 6 | 15.81 | 5.60 | 3.67 | 88.01 |
| 7 | 16.38 | 5.41 | 5.24 | 125.91 |
| 8 | 17.02 | 5.21 | 100.00 | 2400.78 |
| 9 | 19.28 | 4.60 | 83.53 | 2005.41 |
| 10 | 20.04 | 4.43 | 21.49 | 516.04 |
| 11 | 21.02 | 4.22 | 5.74 | 137.75 |
| 12 | 21.78 | 4.08 | 4.19 | 100.66 |
| 13 | 22.08 | 4.02 | 20.65 | 495.86 |
| 14 | 22.49 | 3.95 | 59.49 | 1428.19 |
| 15 | 22.58 | 3.93 | 35.41 | 850.00 |
| 16 | 23.25 | 3.82 | 24.20 | 580.97 |
| 17 | 23.69 | 3.75 | 13.06 | 313.50 |
| 18 | 23.87 | 3.73 | 24.08 | 578.16 |
| 19 | 24.12 | 3.69 | 10.14 | 243.33 |
| 20 | 25.26 | 3.52 | 2.90 | 69.52 |
| 21 | 25.52 | 3.49 | 3.51 | 84.28 |
| 22 | 26.25 | 3.39 | 12.91 | 309.86 |
| 23 | 26.75 | 3.33 | 5.78 | 138.84 |
| 24 | 27.23 | 3.27 | 6.84 | 164.29 |
| 25 | 27.89 | 3.20 | 9.45 | 226.94 |
| 26 | 28.33 | 3.15 | 4.52 | 108.51 |
| 27 | 29.42 | 3.03 | 2.25 | 53.99 |
| 28 | 30.40 | 2.94 | 3.09 | 74.14 |
| 29 | 31.72 | 2.82 | 4.49 | 107.79 |
| 30 | 31.79 | 2.81 | 4.20 | 100.82 |
| 31 | 32.21 | 2.78 | 1.99 | 47.78 |
| 32 | 32.63 | 2.74 | 2.43 | 58.30 |
| 33 | 35.59 | 2.52 | 7.25 | 173.97 |
| 34 | 35.89 | 2.50 | 5.34 | 128.24 |
| 35 | 39.53 | 2.28 | 1.70 | 40.74 |

A summary of the DVS isotherm analysis of Form F is found below in Table 9.

TABLE 9
DVS Isotherm Analysis of Form F

| | |
|---|---|
| Target % | Change In Mass (%) - ref |

| | P/Po | Sorption | Desorption | Hysteresis |
|---|---|---|---|---|
| Cycle 1 | 0.0 | 0.006 | 0.006 | |
| | 10.0 | 0.173 | 0.301 | 0.128 |
| | 20.0 | 0.324 | 0.458 | 0.134 |
| | 30.0 | 0.455 | 0.566 | 0.111 |
| | 40.0 | 0.574 | 0.649 | 0.075 |
| | 50.0 | 0.690 | — | — |
| | 60.0 | 0.813 | — | — |
| | 70.0 | 0.964 | — | — |
| | 80.0 | 1.160 | — | — |
| | 90.0 | 1.601 | — | — |
| | 95.0 | 1.911 | — | — |
| Cycle 2 | 0.0 | 0.156 | 0.156 | |
| | 10.0 | 0.363 | 0.718 | 0.355 |
| | 20.0 | 0.565 | 1.060 | 0.495 |
| | 30.0 | 0.752 | 1.291 | 0.539 |
| | 40.0 | 0.927 | 1.459 | 0.532 |
| | 50.0 | — | 1.595 | — |
| | 60.0 | — | 1.711 | — |
| | 70.0 | — | 1.813 | — |
| | 80.0 | — | 1.901 | — |
| | 90.0 | — | 1.964 | — |
| | 95.0 | — | 1.911 | — |

Temperature 24.9° C.
MRef: 19.4778 from Custom Mass

### Form G

Compound 1 Form A was dissolved in DMSO. The clear solution was added to water quickly (1:3 DMSO:water). The precipitate was collected filtration and Form G was then characterized by XRPD (FIG. 7, Table 10).

A summary of the XRPD peaks of Form G is found below in Table 10.

TABLE 10
XRPD Peaks of Form G

| No. | 2-Theta | d(Å) | Relative Intensity (%) | Intensity |
|---|---|---|---|---|
| 1 | 6.18 | 14.29 | 24.20 | 65.64 |
| 2 | 11.38 | 7.77 | 14.22 | 38.57 |
| 3 | 12.61 | 7.01 | 80.03 | 217.04 |
| 4 | 13.37 | 6.62 | 30.00 | 81.35 |
| 5 | 18.96 | 4.68 | 29.76 | 80.70 |
| 6 | 21.22 | 4.18 | 43.35 | 117.55 |
| 7 | 23.52 | 3.78 | 100.00 | 271.18 |
| 8 | 26.22 | 3.40 | 23.87 | 64.72 |
| 9 | 26.46 | 3.37 | 22.74 | 61.67 |
| 10 | 28.02 | 3.18 | 11.79 | 31.98 |
| 11 | 31.40 | 2.85 | 17.01 | 46.12 |
| 12 | 36.04 | 2.49 | 10.68 | 28.97 |

### Form H

Compound 1 Form H was observed upon heating of Compound 1 Form A to 110° C. during VT-XRPD or at 0% RH during VH-XRPD. Form H was then characterized by XRPD (FIG. 8, Table 11).

A summary of the XRPD peaks of Form H is found below in Table 11.

TABLE 11
XRPD Peaks of Form H

| No. | 2-Theta | d(Å) | Relative Intensity (%) | Intensity |
|---|---|---|---|---|
| 1 | 5.73 | 15.42 | 100.00 | 4780.86 |
| 2 | 5.96 | 14.83 | 45.76 | 2187.60 |
| 3 | 7.07 | 12.50 | 38.69 | 1849.68 |
| 4 | 9.23 | 9.57 | 1.66 | 79.46 |
| 5 | 11.38 | 7.77 | 25.00 | 1195.36 |
| 6 | 11.73 | 7.54 | 64.89 | 3102.28 |
| 7 | 14.11 | 6.27 | 4.39 | 209.86 |
| 8 | 16.54 | 5.36 | 16.58 | 792.59 |
| 9 | 17.21 | 5.15 | 37.16 | 1776.80 |
| 10 | 17.82 | 4.97 | 12.66 | 605.25 |
| 11 | 18.43 | 4.81 | 8.76 | 418.78 |
| 12 | 19.07 | 4.65 | 4.49 | 214.65 |
| 13 | 19.66 | 4.51 | 7.79 | 372.38 |
| 14 | 20.13 | 4.41 | 1.06 | 50.74 |
| 15 | 20.57 | 4.31 | 2.11 | 100.87 |
| 16 | 21.38 | 4.15 | 6.47 | 309.31 |
| 17 | 22.02 | 4.03 | 7.45 | 356.30 |
| 18 | 22.98 | 3.87 | 44.17 | 2111.65 |
| 19 | 24.04 | 3.70 | 32.72 | 1564.10 |
| 20 | 25.44 | 3.50 | 2.17 | 103.93 |
| 21 | 26.00 | 3.42 | 3.48 | 166.52 |
| 22 | 27.12 | 3.28 | 1.04 | 49.79 |
| 23 | 28.20 | 3.16 | 2.72 | 129.88 |
| 24 | 30.31 | 2.95 | 0.87 | 41.62 |

### Form I

Compound 1 Form I was observed upon heating of Compound 1 Form A to 252° C. during VT-XRPD or at 0% RH during VH-XRPD. Form I was then characterized by XRPD (FIG. 9A, Table 12) and DSC (FIG. 9B).

A summary of the XRPD peaks of Form I is found below in Table 12. A characteristic peak of Form I was identified at 6.2-6.3 °2θ as shown in FIG. 9A and Table 12.

TABLE 12
Characteristic XRPD Peak of Form I

| No. | 2-Theta | d(Å) | Relative Intensity (%) | Intensity |
|---|---|---|---|---|
| 1 | 6.30 | 14.02 | 17.50 | 110.12 |

### Form J

Compound 1 Form J was formed from acetic acid and acetone. Form J was then characterized by XRPD (FIG. 10A, Table 13) and DSC (FIG. 10B).

A summary of the XRPD peaks of Form J is found below in Table 13.

TABLE 13
XRPD Peaks of Form J

| No. | 2-Theta | d(Å) | Relative Intensity (%) | Intensity |
|---|---|---|---|---|
| 1 | 5.56 | 15.89 | 36.30 | 617.81 |
| 2 | 6.93 | 12.74 | 35.00 | 595.49 |
| 3 | 9.13 | 9.68 | 8.20 | 139.28 |
| 4 | 11.13 | 7.94 | 62.50 | 1062.77 |
| 5 | 11.63 | 7.60 | 100.00 | 1700.29 |
| 6 | 12.88 | 6.87 | 15.40 | 261.43 |
| 7 | 13.92 | 6.36 | 9.90 | 167.87 |
| 8 | 16.23 | 5.46 | 43.10 | 732.58 |
| 9 | 16.78 | 5.28 | 17.30 | 293.76 |
| 10 | 17.13 | 5.17 | 87.20 | 1482.40 |
| 11 | 17.48 | 5.07 | 3.80 | 64.75 |
| 12 | 17.76 | 4.99 | 37.80 | 642.88 |
| 13 | 18.29 | 4.85 | 46.80 | 795.24 |
| 14 | 19.25 | 4.61 | 6.70 | 113.64 |
| 15 | 19.67 | 4.51 | 2.30 | 39.63 |
| 16 | 20.60 | 4.31 | 2.20 | 36.97 |
| 17 | 20.95 | 4.24 | 19.50 | 332.22 |
| 18 | 21.62 | 4.11 | 5.50 | 92.87 |
| 19 | 22.43 | 3.96 | 38.70 | 658.45 |
| 20 | 22.88 | 3.88 | 18.60 | 315.56 |
| 21 | 23.35 | 3.81 | 1.60 | 27.94 |
| 22 | 23.94 | 3.71 | 11.00 | 187.37 |
| 23 | 25.87 | 3.44 | 2.40 | 41.19 |
| 24 | 26.18 | 3.40 | 2.90 | 48.86 |
| 25 | 27.52 | 3.24 | 1.90 | 32.54 |
| 26 | 27.71 | 3.22 | 4.00 | 67.27 |
| 27 | 28.03 | 3.18 | 3.20 | 53.86 |
| 28 | 29.03 | 3.07 | 2.80 | 48.12 |
| 29 | 30.16 | 2.96 | 1.40 | 23.26 |
| 30 | 31.04 | 2.88 | 0.90 | 14.74 |
| 31 | 31.74 | 2.82 | 2.30 | 39.11 |
| 32 | 32.85 | 2.72 | 2.10 | 36.36 |
| 33 | 37.15 | 2.42 | 0.90 | 14.84 |
| 34 | 38.39 | 2.34 | 1.10 | 18.48 |

### Form K

Compound 1 Form K was formed characterized by XRPD (FIG. 12, Table 14).

A summary of the XRPD peaks of Form K is found below in Table 14.

TABLE 14
XRPD Peaks of Form K

| Peak No. | 2-Theta |
|---|---|
| 1 | 5.3513 |
| 2 | 7.2884 |
| 3 | 11.4278 |
| 4 | 11.9977 |
| 5 | 12.882 |
| 6 | 13.3977 |
| 7 | 14.5904 |
| 8 | 16.099 |
| 9 | 16.87 |
| 10 | 17.3645 |
| 11 | 17.8744 |
| 12 | 18.4377 |
| 13 | 19.0437 |
| 14 | 19.8522 |
| 15 | 21.1407 |
| 16 | 21.4995 |
| 17 | 21.696 |
| 18 | 21.9235 |
| 19 | 22.2138 |
| 20 | 22.941 |
| 21 | 23.2477 |
| 22 | 24.1295 |
| 23 | 24.7961 |
| 24 | 25.069 |
| 25 | 25.75 |
| 26 | 26.328 |
| 27 | 26.7391 |
| 28 | 27.2888 |
| 29 | 27.6477 |
| 30 | 28.5123 |
| 31 | 29.6703 |
| 32 | 32.4993 |
| 33 | 33.8958 |
| 34 | 34.6839 |
| 35 | 35.3228 |
| 36 | 37.3178 |
| 37 | 38.2545 |

### Form L

Compound 1 Form L was formed characterized by XRPD (FIG. 13, Table 15).

A summary of the XRPD peaks of Form L is found below in Table 15.

TABLE 15
XRPD Peaks of Form L

| Peak No. | 2-Theta |
|---|---|
| 1 | 4.2982 |
| 2 | 8.22 |
| 3 | 8.5982 |
| 4 | 12.9214 |
| 5 | 13.3744 |
| 6 | 15.4226 |
| 7 | 15.9129 |
| 8 | 17.188 |
| 9 | 17.7457 |
| 10 | 18.1859 |
| 11 | 18.5716 |
| 12 | 19.1989 |
| 13 | 20.5371 |
| 14 | 21.4779 |
| 15 | 22.1519 |
| 16 | 24.0745 |
| 17 | 24.8749 |
| 18 | 25.7091 |
| 19 | 26.0266 |
| 20 | 26.597 |
| 21 | 27.0546 |
| 22 | 27.4993 |
| 23 | 28.0583 |
| 24 | 29.24 |
| 25 | 30.2214 |
| 26 | 31.1876 |
| 27 | 38.5282 |

### Form M

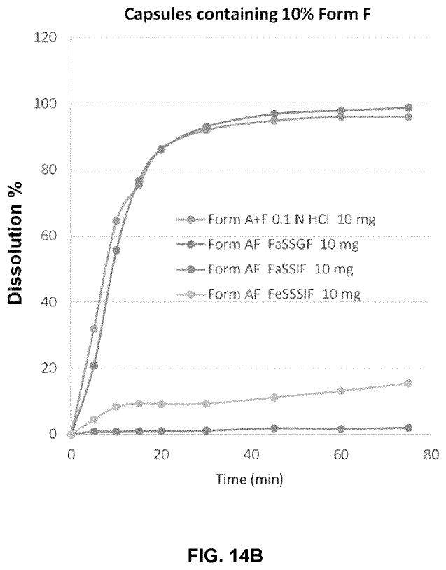

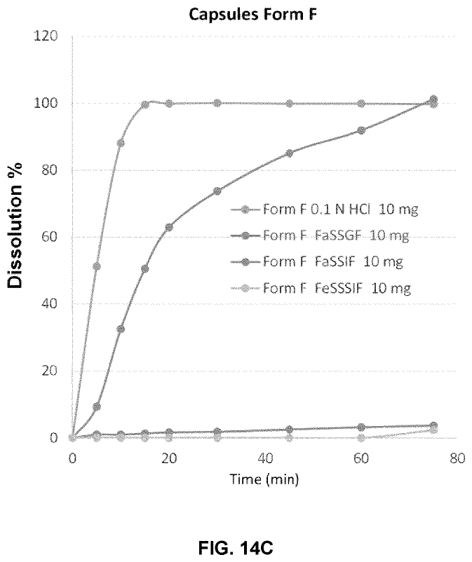

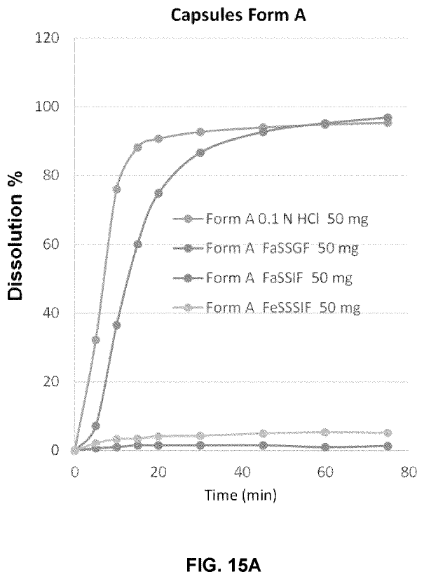

Compound 1 Form M was formed characterized by XRPD (FIG. 14, Table 16).

A summary of the XRPD peaks of Form M is found below in Table 16.

TABLE 16
XRPD Peaks of Form M

| Peak No. | 2-Theta |
|---|---|
| 1 | 7.2633 |
| 2 | 9.2466 |
| 3 | 10.4407 |
| 4 | 11.8012 |
| 5 | 12.7378 |
| 6 | 14.8212 |
| 7 | 16.2096 |
| 8 | 17.4727 |
| 9 | 18.5707 |
| 10 | 18.9932 |
| 11 | 19.9045 |
| 12 | 21.0388 |
| 13 | 22.3391 |
| 14 | 23.5605 |
| 15 | 26.3258 |
| 16 | 29.2081 |
| 17 | 31.17 |
| 18 | 32.5493 |
| 19 | 33.512 |

A summary of the DSC and TGA thermograms of the various forms is found below in Table 17.

TABLE 17
DSC and TGA Thermogram Data for Forms A-M

| Form | DSC Onset Temperatures | TGA |
|---|---|---|
| A | 46.4° C., 230.8° C., | 2.9% @ 120.0° C.; |
| | 241.7° C., 271.9° C. | 1.4% from 120.0° C. to |
| | | 220.0° C. |
| B | 33.1° C., 165.3° C. | 8.4% @ 110.0° C. |
| C | 55.7° C., 132.6° C., | 16.3% at 75.0° C. |
| | 229.2° C., 238.7° C. | |
| D | 51.8° C., 178.5° C. | 5.4% @ 110.0° C.; |
| | | 2.2% from 110.0° C. to 170.0° C. |
| E | 139.4° C., 232.5° C., | 1.8% @ 80.0° C.; |
| | 271.1° C. | 16.4% from 80.0° C. to 140.0° C. |
| F | 270.8° C. | 0.8% @ 110.0° C. |
| G | N/A | N/A |
| H | 260.0° C. | N/A |
| I | 260° C. | N/A |
| J | 83° C., 196° C., 285° C. | N/A |
| K | N/A | N/A |
| L | N/A | N/A |
| M | N/A | N/A |

### CONCLUSION

Various crystalline forms of Compound I were identified: Forms A, B, C, D, E, F, G, H, I, J, K, L, and M. Notably, of these forms, Forms A and F were selected for further pharmaceutical development due to these forms' suitability for large-scale manufacturing, stability, and crystallinity as compared to Forms B, C, D, E, and G. In particular, relative to Forms B, C, D, E, G, H, I, J, K, L, and M, Forms A and F exhibit a high degree of crystallinity (e.g., compare FIGS. 1A and 6A (Forms A and F) to FIGS. 2A and 4A (Forms B and D)) and also offer increased stability as evidenced by DSC thermograms and TGA thermograms (Table 17 and FIGS. 1B and 6B).

### Example 2

In vitro dissolution profiles were analyzed for Form A and Form F. In vitro drug dissolution and release testing can provide insight into a drug's in vivo performance such as bioavailability. Biorelevant media was used to predict the bioavailability of Compound I, which is otherwise a poorly soluble drug.

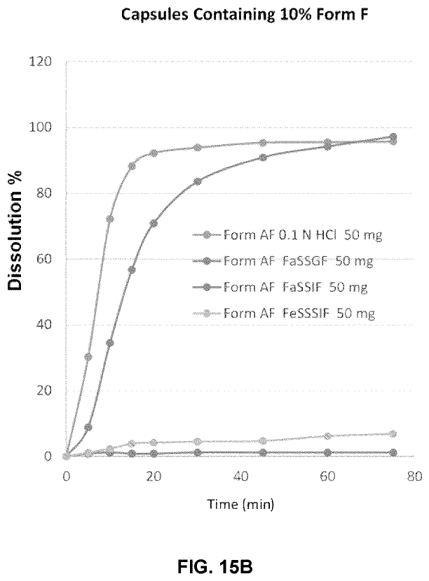

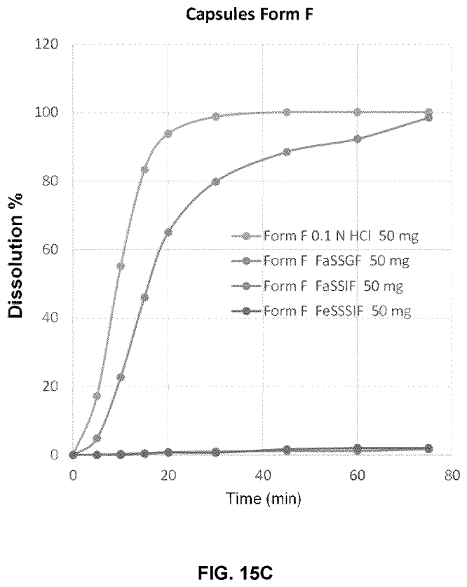

Because Forms A and F were selected for further pharmaceutical development, dissolution tests were performed on these forms. The tests were performed at different pH and in biorelevant media: hydrochloric acid (HCl), fasted state simulated gastric fluid (FaSSGF), fasted state simulated intestinal fluid (FaSSIF), and fed state simulated intestinal fluid (FeSSIF) (75 rpm, 900 mL). Three 10 mg and 50 mg capsules were analyzed as shown below in Table 18. The capsules include those including only Form A, 90% Form A spiked with 10% of Form F, and Form F. The results are shown in FIGS. 14A-14C (10 mg capsules) and FIGS. 15A-15C (50 mg capsules).

TABLE 18
Capsules for Dissolution Analysis

| | 10 mg Capsule | 50 mg Capsule |
|---|---|---|
| 1 | Form A | Form A |
| 2 | Form A, spiked with 10% of | Form A, spiked with 10% of |
| | Form F | Form F |
| 3 | Form F | Form F |

1. A crystalline form of Compound I 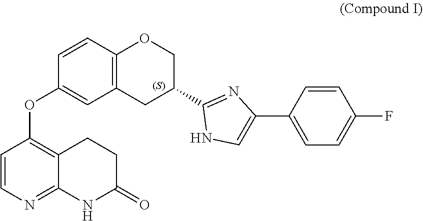
or a salt, solvate, or solvate salt thereof.

2. The crystalline form of claim 1, wherein the crystalline form of Compound I is Form A, Form B, Form C, Form D, Form E, Form F, Form G, Form H, Form J, Form K, Form L, or Form M.

3. The crystalline form of claim 2, wherein the crystalline form of Compound I is Form A.

4. The crystalline form of claim 3, wherein Form A is characterized by peaks in an X-ray diffraction (XPRD) pattern at 5.8±0.2, 11.8±0.2, 16.6±0.2, and 23.1±0.2.

5. The crystalline form of claim 4, wherein Form A is further characterized by at least one peak in an XPRD pattern selected from 5.8±0.2, 11.4±0.2, 17.3±0.2, 17.5±0.2, and 17.9±0.2 °2θ.

6. The crystalline form of claims 3-5, wherein Form A is characterized by peaks in an XPRD pattern at 5.8±0.2, 6.0±0.2, 7.1±0.2, 9.3±0.2, 11.4±0.2, 11.8±0.2, 14.1±0.2, 16.6±0.2, 17.3±0.2, 17.5±0.2, 17.9±0.2, 18.5±0.2, 19.1±0.2, 19.8±0.2, 20.7±0.2, 21.3±0.2, 21.6±0.2, 22.1±0.2, 23.1±0.2, 24.2±0.2, 25.6±0.2, 26.1±0.2, 26.5±0.2, 27.3±0.2, 28.4±0.2, and 35.8±0.2 °2θ.

7. The crystalline form of claims 3-6, characterized by an XRPD pattern substantially similar to that shown in FIG. 1A.

8. The crystalline form of claims 3-7, which exhibits a Differential Scanning Calorimetry (DSC) thermogram comprising a peak at 77±5, 239±5, 243±5, 271±5° C., 285±5° C., or any combination thereof.

9. The crystalline form of claim 8, wherein the peak at 67±5° C. is associated with an enthalpy of 61±5 Joule/gram (J/g); the peak at 239±5° C. is associated with an enthalpy of 78±5 J/g; the peak at 245±5° C. is associated with an enthalpy of 67±5 J/g; and the peak at 277±5° C. is associated with an enthalpy of 84±5 J/g.

10. The crystalline form of claims 3-9, which exhibits a DSC thermogram comprising an endothermic peak with an onset at 46±5, 231±5, or 271±5° C., or any combination thereof.

11. The crystalline form of claims 3-10, which exhibits a DSC thermogram comprising an exothermic peak with an onset at 242±5° C.

12. The crystalline form of claims 3-11, which exhibits a 3% weight loss at a temperature of 120±5° C., a 1% weight loss at 220±5° C. as measured by thermogravimetric (TGA) analysis, or both.

13. The crystalline form of claims 3-12, wherein Compound I is a hydrate.

14. The crystalline form of claim 2, wherein the crystalline form of Compound I is Form B.

15. The crystalline form of claim 14, wherein Form B is characterized by peaks in an XPRD pattern at 12.7±0.2, 13.4±0.2, and 23.4±0.2 °2θ.

16. The crystalline form of claim 15, wherein Form B is further characterized by at least one peak in an XPRD pattern selected from 6.2±0.2, 13.6±0.2, 23.3±0.2, 26.1±0.2, and 26.3±0.2 °2θ.

17. The crystalline form of claims 14-16, wherein Form B is characterized by peaks in an XPRD pattern at 6.2±0.2, 12.7±0.2, 13.4±0.2, 13.6±0.2, 19.0±0.2, 19.8±0.2, 23.3±0.2, 23.4±0.2, 26.1±0.2, 26.3±0.2, and 39.1±0.2 °2θ.

18. The crystalline form of claims 14-17, characterized by an XRPD pattern substantially similar to that shown in FIG. 2A.

19. The crystalline form of claims 14-18, which exhibits a DSC thermogram comprising a peak at 71±5, or 170±5° C., or combination thereof.

20. The crystalline form of claim 19, wherein the peak at 71±5° C. is associated with an enthalpy of 107±5 J/g and the peak at 170±5° C. is associated with an enthalpy of 2±5 J/g.

21. The crystalline form of claims 14-20, which exhibits a DSC thermogram comprising an endothermic peak with an onset at 33±5, or 165±5° C., or combination thereof.

22. The crystalline form of claims 14-21, which exhibits an 8% weight loss at a temperature of 110±5° C. as measured by TGA analysis.

23. The crystalline form of claims 14-22, wherein Compound I is a hydrate.

24. The crystalline form of claim 2, wherein the crystalline form of Compound I is Form C.

25. The crystalline form of claim 24, wherein Form C is characterized by peaks in an XPRD pattern at 16.8±0.2, 23.1±0.2, and 24.2±0.2 °2θ.

26. The crystalline form of claim 25, wherein Form C is further characterized by at least one peak an in XPRD pattern selected from 5.8±0.2, 11.5±0.2, 17.2±0.2, 17.5±0.2, and 18.4±0.2 °2θ.

27. The crystalline form of claims 24-26, wherein Form C is characterized by peaks in an XPRD pattern at 3.6±0.2, 4.2±0.2, 5.8±0.2, 7.1±0.2, 9.4±0.2, 11.5±0.2, 11.7±0.2, 12.8±0.2, 14.1±0.2, 16.8±0.2, 17.2±0.2, 17.5±0.2, 17.9±0.2, 18.2±0.2, 18.4±0.2, 19.0±0.2, 19.6±0.2, 20.4±0.2, 21.3±0.2, 22.1±0.2, 22.6±0.2, 23.1±0.2, 23.4±0.2, 24.2±0.2, 25.7±0.2, 26.2±0.2, 26.6±0.2, 28.5±0.2, 30.3±0.2, 31.0±0.2, 32.4±0.2, 34.0±0.2, and 36.2±0.2 °2θ.

28. The crystalline form of claims 24-27 characterized by an XRPD pattern substantially similar to that shown in FIG. 3A.

29. The crystalline form of claims 24-28, which exhibits a DSC thermogram comprising a peak at 92±5, 154±5, 231±5, or 242±5° C., or any combination thereof.

30. The crystalline form of claim 29, wherein the peak at 92±5° C. is associated with an enthalpy of 301±5 J/g; the peak at 154±5° C. is associated with an enthalpy of 63±5 J/g; the peak at 231±5° C. is associated with an enthalpy of 14±5 J/g; and the peak at 242±5° C. is associated with an enthalpy of 14±5 J/g.

31. The crystalline form of claims 28-30, which exhibits a DSC thermogram comprising an endothermic peak with an onset at 56±5, 133±5, 229±5, or 239±5° C., or any combination thereof.

32. The crystalline form of claims 28-31, which exhibits a 16% weight loss at a temperature of 75±5° C. as measured by TGA analysis.

33. The crystalline form of claims 28-32, wherein Compound I is a solvate.

34. The crystalline form of claim 33, wherein the solvate is a dimethyl sulfoxide (DMSO) solvate.

35. The crystalline form of claim 2, wherein the crystalline form of Compound I is Form D.

36. The crystalline form of claim 35, wherein Form D is characterized by peaks in an XPRD pattern at 12.5±0.2 and 23.4±0.2 and 23.1±0.2 °2θ.

37. The crystalline form of claim 36, wherein Form D is further characterized by at least one peak in an XPRD pattern selected from 11.5±0.2 and 21.3±0.2, °2θ.

38. The crystalline form of claims 35-37, wherein Form D is characterized by peaks in an XPRD pattern at 5.8±0.2, 6.0±0.2, 7.2±0.2, 8.9±0.2, 11.7±0.2, 11.8±0.2, 12.5±0.2, 13.0±0.2, 16.9±0.2, 17.3±0.2, 17.8±0.2, 18.5±0.2, 19.1±0.2, 19.7±0.2, 21.3±0.2, 23.1±0.2, 23.4±0.2, 24.1±0.2, 26.2±0.2, and 26.4±0.2 °2θ.

39. The crystalline form of claims 35-38, characterized by an XRPD pattern substantially similar to that shown in FIG. 4A.

40. The crystalline form of claims 35-39, which exhibits a DSC thermogram comprising a peak at 84±5, or 191±5° C., or combination thereof.

41. The crystalline form of claim 40, wherein the peak at 84±5° C. is associated with an enthalpy of 300±5 J/g and the peak at 191±5° C. is associated with an enthalpy of 8±5 J/g.

42. The crystalline form of claims 35-41, which exhibits a DSC thermogram comprising an endothermic peak with an onset at 52±5, or 179±5° C., or combination thereof.

43. The crystalline form of claims 35-42, which exhibits a 5% weight loss at a temperature of 110±5° C., a 2% weight loss at 170±5° C. as measured by TGA analysis, or both.

44. The crystalline form of claims 35-43, wherein Compound I is a solvate.

45. The crystalline form of claim 44, wherein the solvate is a dimethylformamide (DMF) solvate.

46. The crystalline form of claim 2, wherein the crystalline form of Compound I is Form E.

47. The crystalline form of claim 46, wherein Form E is characterized by peaks in an XPRD pattern at 5.8±0.2, 17.4±0.2, and 22.9±0.2 °2θ.

48. The crystalline form of claim 47, wherein Form E is further characterized by at least one peak an in XPRD pattern selected from 11.6±0.2, 16.7±0.2, 17.1±0.2, 23.3±0.2, and 24.0±0.2 °2θ.

49. The crystalline form of claims 46-48, wherein Form E is characterized by peaks in an XPRD pattern at 5.8±0.2, 7.0±0.2, 9.4±0.2, 11.4±0.2, 11.6±0.2, 14.1±0.2, 16.7±0.2, 17.1±0.2, 17.4±0.2, 17.8±0.2, 18.1±0.2, 18.3±0.2, 18.8±0.2, 19.4±0.2, 20.2±0.2, 20.9±0.2, 21.1±0.2, 21.8±0.2, 22.1±0.2, 22.9±0.2, 23.3±0.2, 24.0±0.2, 25.4±0.2, 25.9±0.2, 26.3±0.2, 28.3±0.2, 33.3±0.2, and 35.7±0.2 °2θ.

50. The crystalline form of claims 46-49 characterized by an XRPD pattern substantially similar to that shown in FIG. 5A.

51. The crystalline form of claims 46-50, which exhibits a DSC thermogram comprising a peak at 176±5, 239±5, or 276±5° C., or any combination thereof.

52. The crystalline form of claim 51, wherein the peak at 176±5° C. is associated with an enthalpy of 75±5 J/g; the peak at 239±5° C. is associated with an enthalpy of 32±5 J/g; and the peak at 276±5° C. is associated with an enthalpy of 17±5 J/g.

53. The crystalline form of claims 46-52, which exhibits a DSC thermogram comprising an endothermic peak with an onset at 139±5, 233±5, or 271±5° C., or any combination thereof.

54. The crystalline form of claims 46-53, which exhibits a 2% weight loss at a temperature of 80±5° C., a 16% weight loss at 140±5° C. as measured by TGA analysis, or both.

55. The crystalline form of claims 46-54, wherein Compound I is a solvate.

56. The crystalline form of claim 2, wherein the crystalline form of Compound I is Form F.

57. The crystalline form of claim 56, wherein Form F is characterized by peaks in an XPRD pattern at 13.0±0.2, 17.0±0.2, and 19.3±0.2 °2θ.

58. The crystalline form of claim 57, wherein Form F is further characterized by at least one peak in an XPRD pattern selected from 20.0±0.2, 22.5±0.2, 22.6±0.2, 23.3±0.2, and 23.9±0.2 °2θ.

59. The crystalline form of claims 56-58, wherein Form F is characterized by peaks in an XPRD pattern at 11.8±0.2, 13.0±0.2, 13.9±0.2, 14.2±0.2, 14.5±0.2, 15.8±0.2, 16.4±0.2, 17.0±0.2, 19.3±0.2, 20.0±0.2, 21.0±0.2, 21.8±0.2, 22.1±0.2, 22.5±0.2, 22.6±0.2, 23.3±0.2, 23.7±0.2, 23.9±0.2, 24.1±0.2, 25.3±0.2, 25.5±0.2, 26.3±0.2, 26.8±0.2, 27.2±0.2, 27.9±0.2, 28.3±0.2, 29.4±0.2, 30.4±0.2, 31.7±0.2, 31.8±0.2, 32.2±0.2, 32.6±0.2, 35.6±0.2, 35.9±0.2, and 39.5±0.2 °2θ.

60. The crystalline form of claims 56-59, characterized by an XRPD pattern substantially similar to that shown in FIG. 6A.

61. The crystalline form of claims 56-60, which exhibits a DSC thermogram comprising a peak at 277±5° C.

62. The crystalline form of claim 61, wherein the peak at 277±5° C. is associated with an enthalpy of 113±5 J/g.

63. The crystalline form of claims 56-62, which exhibits a DSC thermogram comprising an endothermic peak with an onset at 269±5° C.

64. The crystalline form of claims 56-63, which exhibits a weight loss of less than 1% at a temperature of 100±5° C. as measured by TGA.

65. The crystalline form of claims 56-64, wherein Compound I is anhydrous.

66. The crystalline form of claim 2, wherein the crystalline form of Compound I is Form G.

67. The crystalline form of claim 66, wherein Form G is characterized by peaks in an XPRD pattern at 12.6±0.2, 21.2±0.2, and 23.5±0.2 °2θ.

68. The crystalline form of claim 67, wherein Form G is further characterized by at least one peak in an XPRD pattern selected from 6.2±0.2, 13.4±0.2, 19.0±0.2, 26.2±0.2, and 26.5±0.2 °2θ.

69. The crystalline form of claims 66-68, wherein Form G is characterized by peaks in an XPRD pattern at 6.2±0.2, 11.4±0.2, 12.6±0.2, 13.4±0.2, 19.0±0.2, 21.2±0.2, 23.5±0.2, 26.2±0.2, 26.5±0.2, 28.0±0.2, 31.4±0.2, and 36.0±0.2 °2θ.

70. The crystalline form of claims 66-69, characterized by an XRPD pattern substantially similar to that shown in FIG. 7A.

71. The crystalline form of claims 66-70, wherein Compound I is a solvate.

72. The crystalline form of claim 71, wherein the solvate is a DMSO solvate.

73. The crystalline form of claim 2, wherein the crystalline form of Compound I is Form H.

74. The crystalline form of claim 73, wherein Form H is characterized by peaks in an XRPD pattern at 5.7±0.2, 6.0±0.2, and 11.7±0.2 °2θ.

75. The crystalline form of claim 74, wherein Form H is further characterized by at least one peak an in XPRD pattern selected from 7.1±0.2, 11.4±0.2, 17.2±0.2, 23.0±0.2, and 24.0±0.2 °2θ.

76. The crystalline form of claims 73-75, wherein Form H is characterized by peaks in an XPRD pattern at 5.7±0.2, 6.0±0.2, 7.1±0.2, 9.2±0.2, 11.4±0.2, 11.7±0.2, 14.1±0.2, 16.5±0.2, 17.2±0.2, 17.8±0.2, 18.4±0.2, 19.1±0.2, 19.7±0.2, 20.1±0.2, 20.6±0.2, 21.4±0.2, 22.0±0.2, 23.0±0.2, 24.0±0.2, 25.4±0.2, 26.0±0.2, 27.1±0.2, 28.2±0.2, and 30.3±0.2 °2θ.

77. The crystalline form of claims 73-76, characterized by an XRPD pattern substantially similar to that shown in FIG. 8A.

78. The crystalline form of claims 73-77, wherein Compound I is anhydrous.

79. The crystalline form of claim 2, wherein the crystalline form of Compound I is Form J.

80. The crystalline form of claim 79, wherein Form J is characterized by peaks in an XRPD pattern at 11.1±0.2, 11.6±0.2, and 17.1±0.2 °2θ.

81. The crystalline form of claim 80, further characterized by at least one peak in an XRPD pattern selected from 5.6±0.2, 6.9±0.2, 16.2±0.2, 17.8±0.2, and 18.3±0.2 °2θ.

82. The crystalline form of claims 79-81, wherein Form J is characterized by peaks in an XPRD pattern at 5.6±0.2, 6.9±0.2, 9.1±0.2, 11.1±0.2, 11.6±0.2, 12.9±0.2, 13.9±0.2, 16.2±0.2, 16.8±0.2, 17.1±0.2, 17.5±0.2, 17.8±0.2, 18.29±0.2, 19.2±0.2, 19.7±0.2, 20.6±0.2, 21.0±0.2, 21.6±0.2, 22.4±0.2, 22.9±0.2, 23.4±0.2, 23.9±0.2, 25.9±0.2, 26.2±0.2, 27.5±0.2, 27.7±0.2, 28.0±0.2, 29.0±0.2, 30.1±0.2, 31.0±0.2, 31.7±0.2, 32.9±0.2, 37.2±0.2, and 38.4±0.2 °2θ.

83. The crystalline form of claims 79-81, characterized by an XRPD pattern substantially similar to that shown in FIG. 10A.

84. The crystalline form of claims 79-81, which exhibits a which exhibits a DSC thermogram comprising a peak at 83±5° C., 196±5° C., and 285±5° C.

85. The crystalline form of claim 2, wherein the crystalline form of Compound I is Form K.

86. The crystalline form of claim 85, wherein Form K is characterized by peaks in an XRPD pattern at 5.4±0.2, 13.4±0.2, and 17.9±0.2 °2θ.

87. The crystalline form of claim 85 or 86, characterized by peaks in an XRPD pattern at 5.5±0.2, 7.3±0.2, 11.4±0.2, 12.0±0.2, 12.9±0.2, 13.4±0.2, 14.6±0.2, 16.1±0.2, 16.9±0.2, 17.4±0.2, 17.9±0.2, 18.4±0.2, 19.0±0.2, 19.9±0.2, 21.1±0.2, 21.5±0.2, 21.7±0.2, 21.9±0.2, 22.2±0.2, 22.9±0.2, 23.2±0.2, 24.1±0.2, 24.8±0.2, 25.1±0.2, 27.8±0.2, 26.3±0.2, 26.7±0.2, 27.3±0.2, 27.6±0.2, 28.5±0.2, 29.7±0.2, 32.5±0.2, 33.9±0.2, 34.7±0.2, 35.3±0.2, 37.3±0.2, and 38.3±0.2 °2θ.

88. The crystalline form of claims 85-87, characterized by an XRPD pattern substantially similar to that shown in FIG. 11.

89. The crystalline form of claim 2, wherein the crystalline form of Compound I is Form L.

90. The crystalline form of claim 89, wherein Form L is characterized by peaks in an XRPD pattern at 4.3±0.2, 8.2±0.2, and 8.6±0.2 °2θ.

91. The crystalline form of claim 89 or 90, characterized by peaks in an XRPD pattern at 4.3±0.2, 8.2±0.2, 8.6±0.2, 12.9±0.2, 13.4±0.2, 15.4±0.2, 15.9±0.2, 17.2±0.2, 17.7±0.2, 18.2±0.2, 18.6±0.2, 19.2±0.2, 20.5±0.2, 21.5±0.2, 22.2±0.2, 24.1±0.2, 24.9±0.2, 25.7±0.2, 26.0±0.2, 26.6±0.2, 27.1±0.2, 27.5±0.2, 28.1±0.2, 29.2±0.2, 30.2±0.2, 31.2±0.2, and 38.5±0.2 °2θ.

92. The crystalline form of claims 89-91, characterized by an XRPD pattern substantially similar to that shown in FIG. 12.

93. The crystalline form of claim 2, wherein the crystalline form of Compound I is Form M.

94. The crystalline form of claim 93, wherein Form M is characterized by peaks in an XRPD pattern at 7.3±0.2, 10.4±0.2, and 12.7±0.2 °2θ.

95. The crystalline form of claim 89 or 90, characterized by peaks in an XRPD pattern at 7.3±0.2, 9.2±0.2, 10.4±0.2, 11.8±0.2, 12.7±0.2, 14.8±0.2, 16.2±0.2, 17.5±0.2, 18.6±0.2, 19.0±0.2, 19.9±0.2, 21.0±0.2, 22.3±0.2, 23.6±0.2, 29.2±0.2, 31.2±0.2, 32.5±0.2, and 33.5±0.2 °2θ.

96. The crystalline form of claims 93-95, characterized by an XRPD pattern substantially similar to that shown in FIG. 13.

97. A composition comprising a crystalline form of any one of claims 1-96 and a pharmaceutically acceptable excipient.

98. A method of treating a disease or condition comprising administering a therapeutically effective amount of a crystalline form of any one of claims 1-96 to a subject in need thereof.

99. The method of claim 99, wherein the disease or condition is implicated by abnormal activity of one or more RAF kinase.

100. The method of claim 100, wherein the one or more RAF kinases is B-RAF.

101. The method of any one of claims 99-101, wherein the disease or condition is cancer.

102. The method of claim 102, wherein the cancer is Barret's adenocarcinoma; biliary tract carcinomas; breast cancer; cervical cancer; cholangiocarcinoma; central nervous system tumors; primary CNS tumors; glioblastomas, astrocytomas; glioblastoma multiforme; ependymomas; secondary CNS tumors (metastases to the central nervous system of tumors originating outside of the central nervous system); brain tumors; brain metastases; colorectal cancer; large intestinal colon carcinoma; gastric cancer; carcinoma of the head and neck; squamous cell carcinoma of the head and neck; acute lymphoblastic leukemia; acute myelogenous leukemia (AML); myelodysplastic syndromes; chronic myelogenous leukemia; Hodgkin's lymphoma; non-Hodgkin's lymphoma; megakaryoblastic leukemia; multiple myeloma; erythroleukemia; hepatocellular carcinoma; lung cancer; small cell lung cancer; non-small cell lung cancer; ovarian cancer; endometrial cancer; pancreatic cancer; pituitary adenoma; prostate cancer; renal cancer; metastatic melanoma or thyroid cancer.

103. The method of claim 102, wherein the cancer is melanoma, non-small cell cancer, colorectal cancer, or pancreatic cancer.
